SEQUENCE LISTING (16 sequences)

SEQ ID NO: 1 - DNA, 1599 bp, Homo sapiens
tctgccccaccctgtcctctggaacctctgcgagatttagaggaaagaaccagttttcaggcggattgcctcagatcacactatctccacttgcccagccctgtggaagattagcggccatgtattccaatgtgataggaactgtaacctctggaaaaaggaaggtttatcttttgtccttgctgctcattggcttctgggactgcgtgacctgtcacgggagccctgtggacatctgcacagccaagccgcgggacattcccatgaatcccatgtgcatttaccgctccccggagaagaaggcaactgaggatgagggctcagaacagaagatcccggaggccaccaaccggcgtgtctgggaactgtccaaggccaattcccgctttgctaccactttctatcagcacctggcagattccaagaatgacaatgataacattttcctgtcacccctgagtatctccacggcttttgctatgaccaagctgggtgcctgtaatgacaccctccagcaactgatggaggtatttaagtttgacaccatatctgagaaaacatctgatcagatccacttcttctttgccaaactgaactgccgactctatcgaaaagccaacaaatcctccaagttagtatcagccaatcgcctttttggagacaaatcccttaccttcaatgagacctaccaggacatcagtgagttggtatatggagccaagctccagcccctggacttcaaggaaaatgcagagcaatccagagcggccatcaacaaatgggtgtccaataagaccgaaggccgaatcaccgatgtcattccctcggaagccatcaatgagctcactgttctggtgctggttaacaccatttacttcaagggcctgtggaagtcaaagttcagccctgagaacacaaggaaggaactgttctacaaggctgatggagagtcgtgttcagcatctatgatgtaccaggaaggcaagttccgttatcggcgcgtggctgaaggcacccaggtgcttgagttgcccttcaaaggtgatgacatcaccatggtcctcatcttgcccaagcctgagaagagcctggccaaggtagagaaggaactcaccccagaggtgctgcaagagtggctggatgaattggaggagatgatgctggtggtccacatgccccgcttccgcattgaggacggcttcagtttgaaggagcagctgcaagacatgggccttgtcgatctgttcagccctgaaaagtccaaactcccaggtattgttgcagaaggccgagatgacctctatgtctcagatgcattccataaggcatttcttgaggtaaatgaagaaggcagtgaagcagctgcaagtaccgctgttgtgattgctggccgttcgctaaaccccaacagggtgactttcaaggccaacaggcctttcctggtttttataagagaagttcctctgaacactattatcttcatgggcagagtagccaacccttgtgttaagtaaaatgttcttattctttgcacctcttcctatttttggtttgtgaacagaagtaaaaataaatacaaactacttccatctcacatta

SEQ ID NO: 2 - DNA, 1545 bp, Macaca mulatta
ggcacgaggaccatctccacttgcccagccctgtggaagattagcgaccatgtattccaatgtgataggaaccgtagcctctggaaaaaggaaggtttatcttctgtccttgctgctcattggcctctgggactgtatgacctgtcacgggagccctgtggacatctgcacagccaagccgcgggacattcccatgaatcccatgtgcatttaccgctccccggagaagaaggcaactgaggatgagggctcagaacagaagatccccgaggccaccaaccggcgcgtctgggaactgtccaaggccaattcccgctttgctaccactttctatcagcacctggcagattccaagaacgacaaggataacattttcctgtcacccctgagtgtctccacggcttttgctatgaccaagctgggtgcctgtaatgacaccctcaagcaactgatggaggtatttaagtttgacaccatatctgagaaaacatctgatcagatccacttcttctttgccaaactgaactgccgactctatcgaaaagccaacaaatcctccaagttagtatcagccaatcgcctttttggagacaaatcccttaccttcaatgagacctaccaggacatcagtgagttggtatacggagccaagctccagcccctggacttcaaggaaaatgcagagcaatccagagcggccatcaacaaatgggtgtccaataagaccgaaggccgaatcaccgatgtcattcccccggaagccatcaacgagctcactgttctggtgctggttaacaccatttacttcaagggcctgtggaagtcaaagtttagccctgagaacacaaggatggaaccgttctacaaggctgatggagagtcgtgttcagcgtctatgatgtaccaggaaggcaagttctgttatcggcgcgtggctgaaggcacccaggtgcttgagttgcccttcaagggtgatgacatcaccatggtgctcatcctgcccaagcctgagaagagcctgaccaaggtggagcaggaactcaccccagaggtgctgcaggagtggctggatgagttggaggagatgatgctggtggttcacatgccccgcttccgcattgaggacggcttcagtttgaaggagcagctgcaagacatgggccttgtcgatctgttcagccctgaaaagtccaaactcccaggtattgttgcagaaggccgggatgacctctatgtctccgatgcattccataaggcatttcttgaggtaaatgaagaaggcagtgaagcagctgcaagtaccgccattgggattgctggccgttcgctaaaccccaacagggtgaccttcaaggccaacaggcctttcctggtttttataagagaagttcctctgaacactattatcttcatgggcagagtagccaacccttgtgtgagctaaactgttcttattctttgtacctcttcctattttggtttgtgaatagaagtaaaaataaatacaactactcccatcttacattaaaaaaaaaaaaaaaaaaa

SEQ ID NO: 3 - DNA, 2171 bp, Mus musculus
ataggtaattttagaaatagatctgatttgtatctgagacattttagtgaagtggtgagatataagacataatcagaagacatatctacctgaagactttaaggggagagctccctcccccacctggcctctggacctctcagatttaggggaaagaaccagttttcggagtgatcgtctcagtcagcaccatctctgtaggagcatcggccatgtattcccctggggcaggaagtggggctgctggtgagaggaagctttgtctcctctctctgctcctcatcggtgccttgggctgtgctatctgtcacggaaaccctgtggacgacatctgcatagcgaagccccgagacatccccgtgaatcccttgtgcatttaccgctcccctgggaagaaggccaccgaggaggatggctcagagcagaaggttccagaagccaccaaccggcgggtctgggaactgtccaaggccaattcgcgatttgccactaacttctaccagcacctggcagactccaagaatgacaacgacaacattttcctgtcacccttgagcatctccactgcttttgctatgaccaagctgggtgcctgtaacgacactctcaagcagctgatggaggtttttaaatttgataccatctccgagaagacatccgaccagatccacttcttctttgccaaactgaactgccgactctatcgaaaagccaacaagtcctctgacttggtatcagccaaccgcctttttggagacaaatccctcaccttcaacgagagctatcaagatgttagtgaggttgtctatggagccaagctccagcccctggacttcaaggagaatccggagcaatccagagtgaccatcaacaactgggtagctaataagactgaaggccgcatcaaagatgtcatcccacagggcgccattaacgagctcactgccctggttctggttaacaccatttacttcaagggcctgtggaagtcaaagttcagccctgagaacacaaggaaggaaccgttctataaggtcgatgggcagtcatgcccagtgcctatgatgtaccaggaaggcaaattcaaataccggcgcgtggcagagggcacccaggtgctagagctgcccttcaagggggatgacatcaccatggtgctcatcctgcccaagcctgagaagagcctggccaaggtggagcaggagctcaccccagagctgctgcaggagtggctggatgagctgtcagagactatgcttgtggtccacatgccccgcttccgcaccgaggatggcttcagtctgaaggagcagctgcaagacatgggcctcattgatctcttcagccctgaaaagtcccaactcccagggatcgttgctggaggcagggacgacctctatgtctccgacgcattccacaaagcatttcttgaggtaaatgaggaaggcagtgaagcagcagcgagtacttctgtcgtgattactggccggtcactgaaccccaatagggtgaccttcaaggccaacaggcccttcctggttcttataagggaagttgcactgaacactattatattcatggggagagtggctaatccttgtgtgaactaaaatattcttaatctttgcaccttttcctactttggtgtttgtgaatagaagtaaaaataaatacgactgccacctcacgagaatggacttttccacttgaagacgagagactggagtacagatgctacaccacttttgggcaagtgaagggggagcagccagccacggtggcacaaacctatatcctggtgcttttgaaggtagaagcagggcggtcaggagttaaggccagttgaggctgggctgcagagtgaaagaccatgtctcaagatggtctttctcctccccaaagtagaaaagaaaaccataaaaacaagaggtaaatatattactatttcatcttagaggatagcaggcatcttgaaagggtagagggaccttaaattctcattattgcccccatactacaaactaaaaaacaaacccgaatcaatctcccataaagacagagattcaaataagagtattaaacgttttatttctcaaaccactcacatgcataatgttcttatacacagtgtcaaaataaagagaaatgcatttttatacaaaaaaaaaaaa

SEQ ID NO: 4 - DNA, 1561 bp, Rattus norvegicus
cggagggattgctcagcactgtctccacggcttctctgcagaagcgtccaccatgtattccccgggaataggaagtgcggttgctggagagaggaagctttgtctcctctctctgctactcattggtgccttgggctgtgctgtctgtcatggaaaccctgtggacgacatctgcatagcgaagccccgagacatccccgtgaaccccatgtgcatttaccgctcccctgcgaagaaggccacggaggaggatgtcctagagcagaaggttccggaagccaccaaccggcgggtctgggaactgtccaaggccaattctcgatttgccactaacttctatcagcacctggcagactccaagaacgacaacgacaacattttcctgtcacccttgagcatctccacggcgtttgctatgaccaagctgggtgcttgtaataacaccctcaagcagctgatggaggtttttaaatttgataccatctccgagaagacatccgaccagatccacttcttctttgccaaactgaactgccgactctatcgaaaagccaacaagtcctctaacttggtgtcagccaaccgcctttttggagacaaatcccttaccttcaatgagagctatcaagacgttagtgagattgtctatggagccaagcttcagcccctggacttcaaggagaatccggagcaatccagagtgaccatcaacaactgggtagctaataagactgaaggccgcatcaaagacgtcatcccccaaggagccattgatgagctcactgccctggtgctggttaacaccatttacttcaagggcctgtggaagtcaaagttcagccctgagaacacaaggaaggaaccattccacaaagttgatgggcagtcatgcctggtgcccatgatgtaccaggaaggcaaattcaaatacaggcgtgtgggagagggtacccaggtgctagagatgcccttcaagggggacgacatcaccatggtgctcatcctgcccaagcctgagaagagcctggctaaggtggagcaggaactcaccccggagctgctgcaggagtggctggatgagctgtcggaggtcatgcttgtggtccacgtgccccgcttccgcatcgaggacagcttcagtctgaaggagcagctgcaagacatgggccttgttgatctcttcagccctgagaagtcccaactcccagggatcattgctgaaggcagggacgacctctttgtctccgatgcattccacaaagcgtttcttgaggtaaatgaggaaggcagtgaagcagcagcgagtacttctgtcgtgattactggccggtcactgaaccccagtagggtgaccttcaaggccaacaggcccttcctggttcttataagggaagtcgcactgaacactattatattcatggggagagtgtctaatccttgtgtgaactaaaatattcttaatctttgcaccttttcctatctcggtgtttgttaatggaagtaaaaataaatatgactgccacctcaaaaaaaaaaaaaaaaaaaaaaaaaaaaaaaaaaa

SEQ ID NO: 5 - DNA, 1599 bp, Homo sapiens
taatgtgagatggaagtagtttgtatttatttttacttctgttcacaaaccaaaaataggaagaggtgcaaagaataagaacattttacttaacacaagggttggctactctgcccatgaagataatagtgttcagaggaacttctcttataaaaaccaggaaaggcctgttggccttgaaagtcaccctgttggggtttagcgaacggccagcaatcacaacagcggtacttgcagctgcttcactgccttcttcatttacctcaagaaatgccttatggaatgcatctgagacatagaggtcatctcggccttctgcaacaatacctgggagtttggacttttcagggctgaacagatcgacaaggcccatgtcttgcagctgctccttcaaactgaagccgtcctcaatgcggaagcggggcatgtggaccaccagcatcatctcctccaattcatccagccactcttgcagcacctctggggtgagttccttctctaccttggccaggctcttctcaggcttgggcaagatgaggaccatggtgatgtcatcacctttgaagggcaactcaagcacctgggtgccttcagccacgcgccgataacggaacttgccttcctggtacatcatagatgctgaacacgactctccatcagccttgtagaacagttccttccttgtgttctcagggctgaactttgacttccacaggcccttgaagtaaatggtgttaaccagcaccagaacagtgagctcattgatggcttccgagggaatgacatcggtgattcggccttcggtcttattggacacccatttgttgatggccgctctggattgctctgcattttccttgaagtccaggggctggagcttggctccatataccaactcactgatgtcctggtaggtctcattgaaggtaagggatttgtctccaaaaaggcgattggctgatactaacttggaggatttgttggcttttcgatagagtcggcagttcagtttggcaaagaagaagtggatctgatcagatgttttctcagatatggtgtcaaacttaaatacctccatcagttgctggagggtgtcattacaggcacccagcttggtcatagcaaaagccgtggagatactcaggggtgacaggaaaatgttatcattgtcattcttggaatctgccaggtgctgatagaaagtggtagcaaagcgggaattggccttggacagttcccagacacgccggttggtggcctccgggatcttctgttctgagccctcatcctcagttgccttcttctccggggagcggtaaatgcacatgggattcatgggaatgtcccgcggcttggctgtgcagatgtccacagggctcccgtgacaggtcacgcagtcccagaagccaatgagcagcaaggacaaaagataaaccttcctttttccagaggttacagttcctatcacattggaatacatggccgctaatcttccacagggctgggcaagtggagatagtgtgatctgaggcaatccgcctgaaaactggttctttcctctaaatctcgcagaggttccagaggacagggtggggcaga

SEQ ID NO: 6 - DNA, 1545 bp, Macaca mulatta
tttttttttttttttttttaatgtaagatgggagtagttgtatttatttttacttctattcacaaaccaaaataggaagaggtacaaagaataagaacagtttagctcacacaagggttggctactctgcccatgaagataatagtgttcagaggaacttctcttataaaaaccaggaaaggcctgttggccttgaaggtcaccctgttggggtttagcgaacggccagcaatcccaatggcggtacttgcagctgcttcactgccttcttcatttacctcaagaaatgccttatggaatgcatcggagacatagaggtcatcccggccttctgcaacaatacctgggagtttggacttttcagggctgaacagatcgacaaggcccatgtcttgcagctgctccttcaaactgaagccgtcctcaatgcggaagcggggcatgtgaaccaccagcatcatctcctccaactcatccagccactcctgcagcacctctggggtgagttcctgctccaccttggtcaggctcttctcaggcttgggcaggatgagcaccatggtgatgtcatcacccttgaagggcaactcaagcacctgggtgccttcagccacgcgccgataacagaacttgccttcctggtacatcatagacgctgaacacgactctccatcagccttgtagaacggttccatccttgtgttctcagggctaaactttgacttccacaggcccttgaagtaaatggtgttaaccagcaccagaacagtgagctcgttgatggcttccgggggaatgacatcggtgattcggccttcggtcttattggacacccatttgttgatggccgctctggattgctctgcattttccttgaagtccaggggctggagcttggctccgtataccaactcactgatgtcctggtaggtctcattgaaggtaagggatttgtctccaaaaaggcgattggctgatactaacttggaggatttgttggcttttcgatagagtcggcagttcagtttggcaaagaagaagtggatctgatcagatgttttctcagatatggtgtcaaacttaaatacctccatcagttgcttgagggtgtcattacaggcacccagcttggtcatagcaaaagccgtggagacactcaggggtgacaggaaaatgttatccttgtcgttcttggaatctgccaggtgctgatagaaagtggtagcaaagcgggaattggccttggacagttcccagacgcgccggttggtggcctcggggatcttctgttctgagccctcatcctcagttgccttcttctccggggagcggtaaatgcacatgggattcatgggaatgtcccgcggcttggctgtgcagatgtccacagggctcccgtgacaggtcatacagtcccagaggccaatgagcagcaaggacagaagataaaccttcctttttccagaggctacggttcctatcacattggaatacatggtcgctaatcttccacagggctgggcaagtggagatggtcctcgtgcc

SEQ ID NO: 7 - DNA, 2171 bp, Mus musculus
ttttttttttttgtataaaaatgcatttctctttattttgacactgtgtataagaacattatgcatgtgagtggtttgagaaataaaacgtttaatactcttatttgaatctctgtctttatgggagattgattcgggtttgttttttagtttgtagtatgggggcaataatgagaatttaaggtccctctaccctttcaagatgcctgctatcctctaagatgaaatagtaatatatttacctcttgtttttatggttttcttttctactttggggaggagaaagaccatcttgagacatggtctttcactctgcagcccagcctcaactggccttaactcctgaccgccctgcttctaccttcaaaagcaccaggatataggtttgtgccaccgtggctggctgctcccccttcacttgcccaaaagtggtgtagcatctgtactccagtctctcgtcttcaagtggaaaagtccattctcgtgaggtggcagtcgtatttatttttacttctattcacaaacaccaaagtaggaaaaggtgcaaagattaagaatattttagttcacacaaggattagccactctccccatgaatataatagtgttcagtgcaacttcccttataagaaccaggaagggcctgttggccttgaaggtcaccctattggggttcagtgaccggccagtaatcacgacagaagtactcgctgctgcttcactgccttcctcatttacctcaagaaatgctttgtggaatgcgtcggagacatagaggtcgtccctgcctccagcaacgatccctgggagttgggacttttcagggctgaagagatcaatgaggcccatgtcttgcagctgctccttcagactgaagccatcctcggtgcggaagcggggcatgtggaccacaagcatagtctctgacagctcatccagccactcctgcagcagctctggggtgagctcctgctccaccttggccaggctcttctcaggcttgggcaggatgagcaccatggtgatgtcatcccccttgaagggcagctctagcacctgggtgccctctgccacgcgccggtatttgaatttgccttcctggtacatcataggcactgggcatgactgcccatcgaccttatagaacggttccttccttgtgttctcagggctgaactttgacttccacaggcccttgaagtaaatggtgttaaccagaaccagggcagtgagctcgttaatggcgccctgtgggatgacatctttgatgcggccttcagtcttattagctacccagttgttgatggtcactctggattgctccggattctccttgaagtccaggggctggagcttggctccatagacaacctcactaacatcttgatagctctcgttgaaggtgagggatttgtctccaaaaaggcggttggctgataccaagtcagaggacttgttggcttttcgatagagtcggcagttcagtttggcaaagaagaagtggatctggtcggatgtcttctcggagatggtatcaaatttaaaaacctccatcagctgcttgagagtgtcgttacaggcacccagcttggtcatagcaaaagcagtggagatgctcaagggtgacaggaaaatgttgtcgttgtcattcttggagtctgccaggtgctggtagaagttagtggcaaatcgcgaattggccttggacagttcccagacccgccggttggtggcttctggaaccttctgctctgagccatcctcctcggtggccttcttcccaggggagcggtaaatgcacaagggattcacggggatgtctcggggcttcgctatgcagatgtcgtccacagggtttccgtgacagatagcacagcccaaggcaccgatgaggagcagagagaggagacaaagcttcctctcaccagcagccccacttcctgccccaggggaatacatggccgatgctcctacagagatggtgctgactgagacgatcactccgaaaactggttctttcccctaaatctgagaggtccagaggccaggtgggggagggagctctccccttaaagtcttcaggtagatatgtcttctgattatgtcttatatctcaccacttcactaaaatgtctcagatacaaatcagatctatttctaaaattacctat

SEQ ID NO: 8 - DNA, 1561 bp, Rattus norvegicus
tttttttttttttttttttttttttttttttttttgaggtggcagtcatatttatttttacttccattaacaaacaccgagataggaaaaggtgcaaagattaagaatattttagttcacacaaggattagacactctccccatgaatataatagtgttcagtgcgacttcccttataagaaccaggaagggcctgttggccttgaaggtcaccctactggggttcagtgaccggccagtaatcacgacagaagtactcgctgctgcttcactgccttcctcatttacctcaagaaacgctttgtggaatgcatcggagacaaagaggtcgtccctgccttcagcaatgatccctgggagttgggacttctcagggctgaagagatcaacaaggcccatgtcttgcagctgctccttcagactgaagctgtcctcgatgcggaagcggggcacgtggaccacaagcatgacctccgacagctcatccagccactcctgcagcagctccggggtgagttcctgctccaccttagccaggctcttctcaggcttgggcaggatgagcaccatggtgatgtcgtcccccttgaagggcatctctagcacctgggtaccctctcccacacgcctgtatttgaatttgccttcctggtacatcatgggcaccaggcatgactgcccatcaactttgtggaatggttccttccttgtgttctcagggctgaactttgacttccacaggcccttgaagtaaatggtgttaaccagcaccagggcagtgagctcatcaatggctccttgggggatgacgtctttgatgcggccttcagtcttattagctacccagttgttgatggtcactctggattgctccggattctccttgaagtccaggggctgaagcttggctccatagacaatctcactaacgtcttgatagctctcattgaaggtaagggatttgtctccaaaaaggcggttggctgacaccaagttagaggacttgttggcttttcgatagagtcggcagttcagtttggcaaagaagaagtggatctggtcggatgtcttctcggagatggtatcaaatttaaaaacctccatcagctgcttgagggtgttattacaagcacccagcttggtcatagcaaacgccgtggagatgctcaagggtgacaggaaaatgttgtcgttgtcgttcttggagtctgccaggtgctgatagaagttagtggcaaatcgagaattggccttggacagttcccagacccgccggttggtggcttccggaaccttctgctctaggacatcctcctccgtggccttcttcgcaggggagcggtaaatgcacatggggttcacggggatgtctcggggcttcgctatgcagatgtcgtccacagggtttccatgacagacagcacagcccaaggcaccaatgagtagcagagagaggagacaaagcttcctctctccagcaaccgcacttcctattcccggggaatacatggtggacgcttctgcagagaagccgtggagacagtgctgagcaatccctccg

SEQ ID NO: 9 - protein, 16 aa, Unknown
  source: /note=“Description of Unknown RFGF peptide”
AAVALLPAVLLALLAP

SEQ ID NO: 10 - protein, 11 aa, Unknown
  source: /note=“Description of Unknown RFGF analogue peptide”
AALLPVLLAAP

SEQ ID NO: 11 - protein, 13 aa, Human immunodeficiency virus
GRKKRRQRRRPPQ

SEQ ID NO: 12 - protein, 16 aa, Drosophila sp.
RQIKIWFQNRRMKWKK

SEQ ID NO: 13 - RNA, 21 nt, Artificial Sequence
  source: /note=“Description of Artificial Sequence Synthetic oligonucleotide”
gguuaacaccauuuacuucaa

SEQ ID NO: 14 - RNA, 23 nt, Artificial Sequence
  source: /note=“Description of Artificial Sequence Synthetic oligonucleotide”
uugaaguaaaugguguuaaccag

SEQ ID NO: 15 - RNA, 23 nt, Artificial Sequence
  source: /note=“Description of Artificial Sequence Synthetic oligonucleotide”
uugaaguaaaugguguuaaccag

SEQ ID NO: 16 - RNA, 21 nt, Artificial Sequence
  source: /note=“Description of Artificial Sequence Synthetic oligonucleotide”
gguuaacaccauuuacuucaa

(19) United States
(12) Patent Application Publication
(10) Pub. No.: US 2022/0079971 A1
(43) Pub. Date: Mar. 17, 2022

(54) SERPINC1 iRNA Compositions and Methods of Use Thereof

(71) Applicant: Genzyme Corporation, Cambridge, MA (US)

(72) Inventor: Akin Akinc, Needham, MA (US)

(73) Assignee: Genzyme Corporation, Cambridge, MA (US)

(21) Appl. No.: 17/422,982

(22) PCT Filed: Jan. 16, 2020
(86) PCT No.: PCT/US2020/013811

§ 371 Date: Jul. 14, 2021

Related U.S. Application Data
(60) Provisional application No. 62/793,020, filed on Jan. 16, 2019.

(51) Int. Cl. A61K 31/713; C12N 15/113; A61K 9/00; A61K 45/06; A61K 47/54

(52) U.S. Cl. CPC A61K 31/713; C12N 15/113; C12N 2310/14; A61K 45/06; A61K 47/549; A61K 9/0019

(57) ABSTRACT

The invention relates to pharmaceutical compositions comprising an iRNA agent, e.g., double stranded ribonucleic acid (dsRNA) agent and methods of using such compositions to treat a bleeding event in a subject having a hemophilia (e.g., with or without inhibitors).

### RELATED APPLICATIONS

This application claims the benefit of priority to U.S. Provisional Application No. 62/793,020, filed on Jan. 16, 2019, the entire contents of which are incorporated herein by reference.

This application is related to International Application No. PCT/US2018/041400, filed on Jul. 10, 2018, U.S. Provisional Patent Application No. 62/530,518, filed on Jul. 10, 2017, U.S. Provisional Patent Application No. 62/599,223, filed on Dec. 15, 2017, U.S. Provisional Patent Application No. 62/614,111, filed on Jan. 5, 2018, and U.S. Provisional Patent Application No. 62/673,424, filed on May 18, 2018. The entire contents of each of the foregoing patent applications are incorporated herein by reference.

This application is also related to U.S. patent application Ser. No. 15/371,300, filed on Dec. 7, 2016, International Application No. PCT/US2016/065245, filed on Dec. 7, 2016, to U.S. Provisional Patent Application No. 62/264,013, filed on Dec. 7, 2015, to U.S. Provisional Patent Application No. 62/315,228, filed on Mar. 30, 2016, to U.S. Provisional Patent Application No. 62/366,304, filed on Jul. 25, 2016, and to U.S. Provisional Patent Application No. 62/429,241, filed on Dec. 2, 2016. The entire contents of each of the foregoing patent applications are hereby incorporated herein by reference.

In addition, this application is related to U.S. Provisional Patent Application No. 61/992,057, filed on May 12, 2014, U.S. Provisional Patent Application No. 62/089,018, filed Dec. 8, 2014, U.S. Provisional Patent Application No. 62/102,281, filed Jan. 12, 2015, and International Application No. PCT/US2015/030337, filed on May 12, 2015. The entire contents of each of the foregoing patent applications are hereby incorporated herein by reference.

This application is also related to U.S. Provisional Patent Application No. 61/638,952, filed on Apr. 26, 2012, U.S. Provisional Patent Application No. 61/669,249, filed on Jul. 9, 2012, U.S. Provisional Patent Application No. 61/734,573, filed on Dec. 7, 2012, U.S. patent application Ser. No. 13/837,129, filed on Mar. 15, 2013, now U.S. Pat. No. 9,127,274, U.S. patent application Ser. No. 14/806,084, filed on Jul. 22, 2015, now U.S. Pat. No. 9,376,680, U.S. patent application Ser. No. 15/070,358, filed on Mar. 15, 2016, U.S. patent application Ser. No. 15/955,873, filed on Apr. 18, 2018, U.S. patent application Ser. No. 16/220,157, filed on Dec. 14, 2018, and International Application No. PCT/US2013/038218, filed on Apr. 25, 2013. This application is also related to International Application No. PCT/US2012/065601, filed on Nov. 16, 2012. The entire contents of each of the foregoing patent applications are hereby incorporated herein by reference.

### SEQUENCE LISTING

The instant application contains a Sequence Listing which has been submitted electronically in ASCII format and is hereby incorporated by reference in its entirety. Said ASCII copy, created on Jan. 7, 2020, is named 117811_03020_SL.TXT and is 20,997 bytes in size.

### BACKGROUND OF THE INVENTION

Serpinc1 is a member of the serine proteinase inhibitor (serpin) superfamily. Serpinc1 is a plasma protease inhibitor that inhibits thrombin as well as other activated serine proteases of the coagulation system, such as factors X, IX, XI, XII and VII and, thus, regulates the blood coagulation cascade. The anticoagulant activity of Serpinc1 is enhanced by the presence of heparin and other related glycosaminoglycans which catalyze the formation of thrombin:antithrombin (TAT) complexes.

Bleeding disorders, either inherited or acquired, are conditions in which there is inadequate blood clotting. For example, hemophilia is a group of hereditary genetic bleeding disorders that impair the body's ability to control blood clotting or coagulation. Hemophilia A is a recessive X-linked genetic disorder involving a lack of functional clotting Factor VIII and represents 80% of hemophilia cases. Hemophilia B is a recessive X-linked genetic disorder involving a lack of functional clotting Factor IX. It comprises approximately 20% of haemophilia cases. Hemophilia C is an autosomal genetic disorder involving a lack of functional clotting Factor XI. Hemophilia C is not completely recessive, as heterozygous individuals also show increased bleeding.

Although at present there is no cure for hemophilia, it can be controlled with regular infusions of the deficient clotting factor, e.g., factor VIII in hemophilia A. However, some hemophiliacs develop antibodies (inhibitors) against the replacement factors given to them and, thus, become refractory to replacement coagulation factor. Accordingly, bleeds in such subjects cannot be properly controlled.

An investigational, once-monthly, subcutaneously administered RNAi therapeutic targeting antithrombin (AT), Fitusiran, has recently been developed for the treatment of hemophilia A and B, with and without inhibitors, and stable pharmaceutical compositions comprising such a therapeutic are needed in the art as alternative treatments for subjects having a bleeding disorder, such as hemophilia.

### SUMMARY OF THE INVENTION

The present invention is based, at least in part, on the discovery of stable pharmaceutical compostions comprising a double stranded ribonucleic acid agent (dsRNA) agent that inhibits the expression of a Serpinc1 gene which have improved satability, efficacy, durability, and ease of administration as compared to other compositions comprising a dsRNA agent that inhibits the expression of a Serpinc1 gene. Such pharmaceutical compositions are useful for treating subjects having a bleeding disorder, such as a hemophilia.

Accordingly, in one aspect the present invention provides a pharmaceutical composition for inhibiting expression of a Serpinc1 gene, comprising a double-stranded ribonucleic acid (dsRNA) agent at a concentration of about 50 mg/mL to about 200 mg/mL and phosphate buffered saline (PBS) at a concentration of about 1 mM to about 10 mM, wherein the pH of the pharmaceutical composition is suitable for subcutaneous administration to a subject, wherein the dsRNA agent has a sense strand consisting of the nucleotide sequence of 5′-GfsgsUfuAfaCfaCfCfAfuUfuAfcUfuCfaAf-3′ (SEQ ID NO:941) and an antisense strand consisting of the nucleotide sequence of 5′-usUfsgAfaGfuAfaAfuggUfgUfuAfaCfcsasg-3′ (SEQ ID NO:960), wherein a, g, c, and u are 2′-O-methyl (2′-OMe) A, G, C, and U; Af, Gf, Cf, and Uf are 2′-fluoro A, G, C, U; and s is a phosphorothioate linkage, and wherein a ligand is conjugated to the 3′ end of the sense strand via a linker, and wherein the ligand and the linker have the following structure:

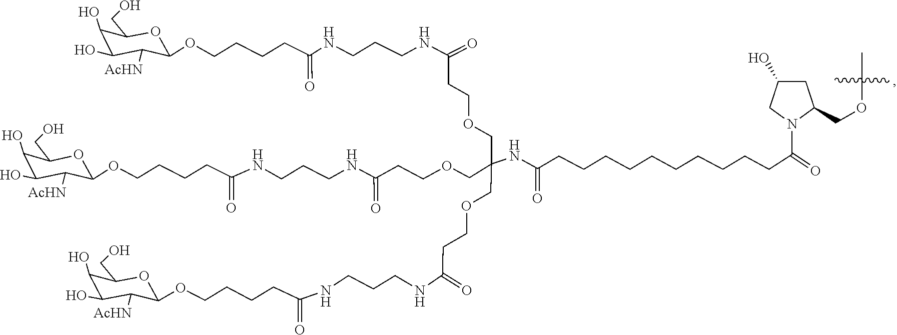 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

wherein the dsRNA agent is in a free acid form.

In another aspect, the present invention provides a pharmaceutical composition for inhibiting expression of a Serpinc1 gene, comprising a double-stranded ribonucleic acid (dsRNA) agent at a concentration of about 50 mg/mL to about 200 mg/mL and phosphate buffered saline (PBS) at a concentration of about 1 mM to about 10 mM, wherein the pH of the pharmaceutical composition is suitable for subcutaneous administration to a subject, wherein the dsRNA agent has a sense strand consisting of the nucleotide sequence of 5′-GfsgsUfuAfaCfaCfCfAfuUfuAfcUfuCfaAf-3′ (SEQ ID NO:941) and an antisense strand consisting of the nucleotide sequence of 5′-usUfsgAfaGfuAfaAfuggUfgUfuAfaCfcsasg-3′ (SEQ ID NO:960), wherein a, g, c, and u are 2′-O-methyl (2′-OMe) A, G, C, and U; Af, Gf, Cf, and Uf are 2′-fluoro A, G, C, U; and s is a phosphorothioate linkage, and wherein a ligand is conjugated to the 3′ end of the sense strand via a linker, and wherein the ligand and the linker have the following structure:

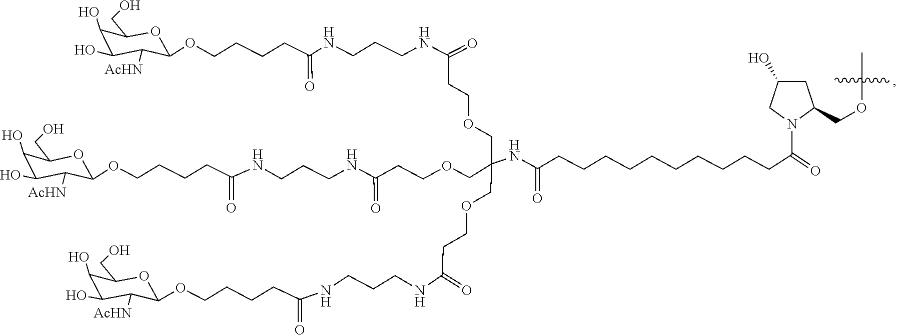 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

wherein the dsRNA agent is in a salt form.

In one aspect the present invention provides a pharmaceutical composition for inhibiting expression of a Serpinc1 gene, comprising a double-stranded ribonucleic acid (dsRNA) agent at a concentration of about 50 mg/mL to about 200 mg/mL and phosphate buffered saline (PBS) at a concentration of about 1 mM to about 10 mM, wherein the pH and the osmolality of the pharmaceutical composition are suitable for subcutaneous administration to a subject, wherein the dsRNA agent has a sense strand consisting of the nucleotide sequence of 5′-GfsgsUfuAfaCfaCfCfAfuUfuAfcUfuCfaAf-3′ (SEQ ID NO:941) and an antisense strand consisting of the nucleotide sequence of 5′-usUfsgAfaGfuAfaAfuggUfgUfuAfaCfcsasg-3′ (SEQ ID NO:960), wherein a, g, c, and u are 2′-O-methyl (2′-OMe) A, G, C, and U; Af, Gf, Cf, and Uf are 2′-fluoro A, G, C, U; and s is a phosphorothioate linkage, and wherein a ligand is conjugated to the 3′ end of the sense strand via a linker, and wherein the ligand and the linker have the following structure:

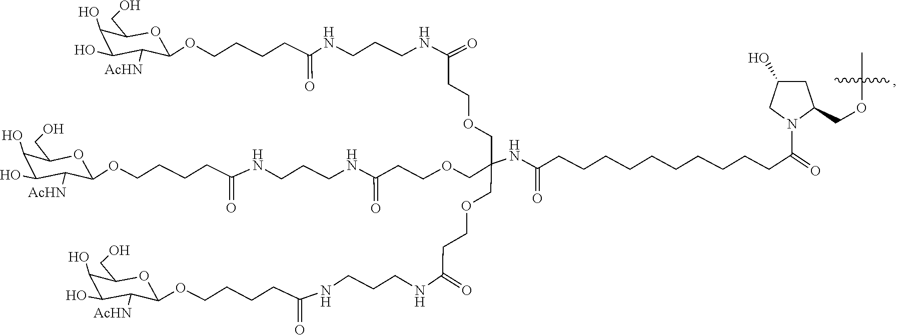 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

wherein the dsRNA agent is in a free acid form.

In another aspect, the present invention provides a pharmaceutical composition for inhibiting expression of a Serpinc1 gene, comprising a double-stranded ribonucleic acid (dsRNA) agent at a concentration of about 50 mg/mL to about 200 mg/mL and phosphate buffered saline (PBS) at a concentration of about 1 mM to about 10 mM, wherein the pH and the osmolality of the pharmaceutical composition are suitable for subcutaneous administration to a subject, wherein the dsRNA agent has a sense strand consisting of the nucleotide sequence of 5′-GfsgsUfuAfaCfaCfCfAfuUfuAfcUfuCfaAf-3′ (SEQ ID NO:941) and an antisense strand consisting of the nucleotide sequence of 5′-usUfsgAfaGfuAfaAfuggUfgUfuAfaCfcsasg-3′ (SEQ ID NO:960), wherein a, g, c, and u are 2′-O-methyl (2′-OMe) A, G, C, and U; Af, Gf, Cf, and Uf are 2′-fluoro A, G, C, U; and s is a phosphorothioate linkage, and wherein a ligand is conjugated to the 3′ end of the sense strand via a linker, and wherein the ligand and the linker have the following structure:

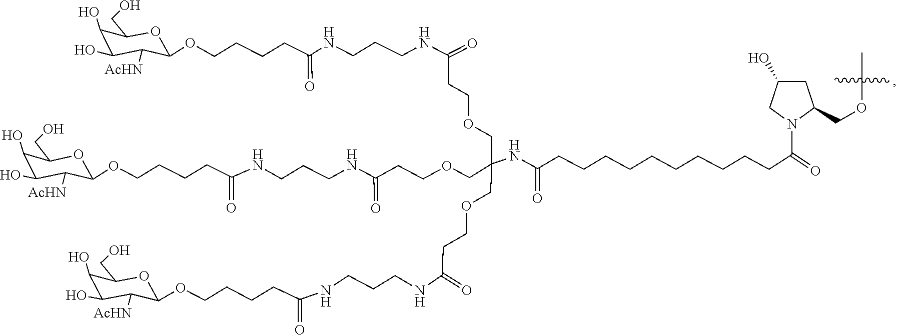 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

wherein the dsRNA agent is in a salt form.

The salt form of the dsRNA may be a sodium salt form.

In one embodiment, substantially all of the phosphodiester and/or phosphorothiotate groups in the agent comprise a sodium counterion. In another embodiment, all of the phosphodiester and/or phosphorothiotate groups in the agent comprise a sodium counterion.

The concentration of PBS in the pharmaceutical composition may be between about 2 mM and about 7 mM; between about 3 to about 6 mM; or about 5 mM.

The pH of the pharmaceutical composition may be between about 5.0 to about 8.0; between about 6.0 to about 8.0; between about 6.5 to about 7.5; or between about 6.8 to about 7.2.

The osmolality of the pharmaceutical composition may be between about 50 and about 400 mOsm/kg; between about 100 and about 400 mOsm/kg; between about 240 and about 390 mOsm/kg; or between about 290 and about 320 mOsm/kg.

The concentration of the dsRNA agent in the pharmaceutical composition may be between about 50 mg/mL and about 150 mg/mL; between about 80 mg/mL and about 110 mg/mL; or about 100 mg/mL.

In one embodiment, the composition is stable for between about 6 months to about 36 months when stored at about 2° C. to about 8° C. In another embodiment, the composition is stable for between about 6 months to about 36 months when stored at about 25° C. and 60% relative humidity (RH). In yet another embodiment, the composition is stable for about 6 months when stored at about 40° C. and 75% relative humidity (RH).

In another embodiment, the composition is stable for up to about 36 months when stored at about 2° C. to about 8° C. In another embodiment, the composition is stable up to about 36 months when stored at about 25° C. and 60% relative humidity (RH). In yet another embodiment, the composition is stable up to about 6 months when stored at about 40° C. and 75% relative humidity (RH).

In one embodiment, the composition comprises not less than (NLT) about 95.0 area % duplex and not more than (NMT) about 5 area % total impurities of duplex as determined by purity non-denaturing IPRP-HPLC.

In one embodiment, the composition comprises not less than (NLT) about 85.0 area % total single strands as determined by purity denaturing AX-HPLC.

In one embodiment, the composition comprises not less than (NLT) about 80.0 area % total single strands as determined by purity denaturing IPRP-HPLC.

The present invention also provides vials and syringes comprising the pharmaceutical compositions of the invention.

The vials may include about 0.5 mL to about 2.0 ml of the pharmaceutical composition; or about 0.8 ml of the pharmaceutical composition.

The syringes of the invention may be a 1 ml syringe; or a 3 ml syringe. In one embodiment, the syringe is a 1 ml single-use syringe.

The syringes of the invention may include a 29 G needle; or a 30 G needle. In one embodiment, the needle is a 29 G needle.

In one aspect, the present invention provides a pharmaceutical composition for inhibiting expression of a Serpinc1 gene, comprising a double-stranded ribonucleic acid (dsRNA) agent at a concentration of about 100 mg/mL and phosphate buffered saline (PBS) at a concentration of about 5 mM, wherein the pH of the pharmaceutical composition is about 6.8 to about 7.2, wherein the dsRNA agent has a sense strand consisting of the nucleotide sequence of 5′-GfsgsUfuAfaCfaCfCfAfuUfuAfcUfuCfaAf-3′ (SEQ ID NO:941) and an antisense strand consisting of the nucleotide sequence of 5′-usUfsgAfaGfuAfaAfuggUfgUfuAfaCfcsasg-3′ (SEQ ID NO:960), wherein a, g, c, and u are 2′-O-methyl (2′-OMe) A, G, C, and U; Af, Gf, Cf, and Uf are 2′-fluoro A, G, C, U; and s is a phosphorothioate linkage, and wherein a ligand is conjugated to the 3′ end of the sense strand via a linker, and wherein the ligand and the linker have the following structure:

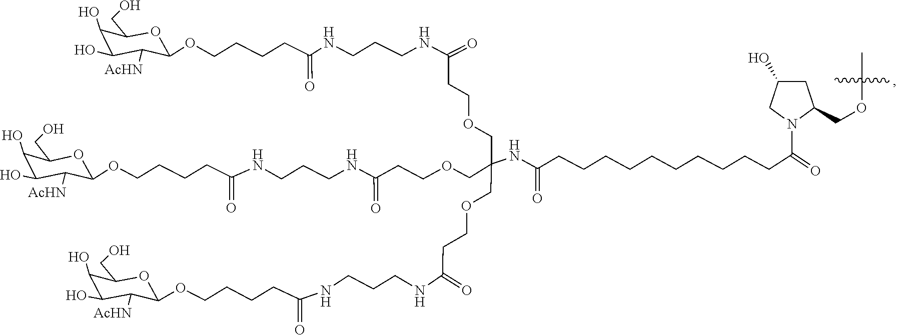 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

wherein the dsRNA agent is in a free acid form.

In another aspect, the present invention provides a pharmaceutical composition for inhibiting expression of a Serpinc1 gene, comprising a double-stranded ribonucleic acid (dsRNA) agent at a concentration of about 106 mg/mL and phosphate buffered saline (PBS) at a concentration of about 5 mM, wherein the pH of the pharmaceutical composition is about 6.8 to about 7.2, wherein the dsRNA agent has a sense strand consisting of the nucleotide sequence of 5′-GfsgsUfuAfaCfaCfCfAfuUfuAfcUfuCfaAf-3′ (SEQ ID NO:941) and an antisense strand consisting of the nucleotide sequence of 5′-usUfsgAfaGfuAfaAfuggUfgUfuAfaCfcsasg-3′ (SEQ ID NO:960), wherein a, g, c, and u are 2′-O-methyl (2′-OMe) A, G, C, and U; Af, Gf, Cf, and Uf are 2′-fluoro A, G, C, U; and s is a phosphorothioate linkage, and wherein a ligand is conjugated to the 3′ end of the sense strand via a linker, and wherein the ligand and the linker have the following structure:

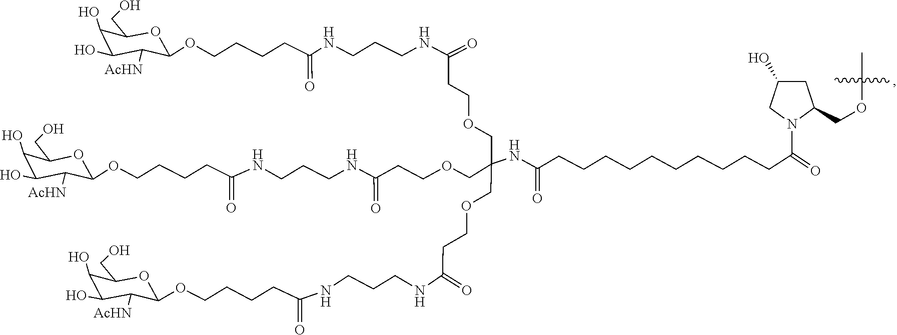 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

wherein the dsRNA agent is in a salt form.

In one aspect, the present invention provides a pharmaceutical composition for inhibiting expression of a Serpinc1 gene, comprising a double-stranded ribonucleic acid (dsRNA) agent at a concentration of about 100 mg/mL and phosphate buffered saline (PBS) at a concentration of about 5 mM, wherein the pH of the pharmaceutical composition is about 6.8 to about 7.2, wherein the osmolality of the pharmaceutical composition is about 300 mOsm/kg, wherein the dsRNA agent has a sense strand consisting of the nucleotide sequence of 5′-GfsgsUfuAfaCfaCfCfAfuUfuAfcUfuCfaAf-3′ (SEQ ID NO:941) and an antisense strand consisting of the nucleotide sequence of 5′-usUfsgAfaGfuAfaAfuggUfgUfuAfaCfcsasg-3′ (SEQ ID NO:960), wherein a, g, c, and u are 2′-O-methyl (2′-OMe) A, G, C, and U; Af, Gf, Cf, and Uf are 2′-fluoro A, G, C, U; and s is a phosphorothioate linkage, and wherein a ligand is conjugated to the 3′ end of the sense strand via a linker, and wherein the ligand and the linker have the following structure:

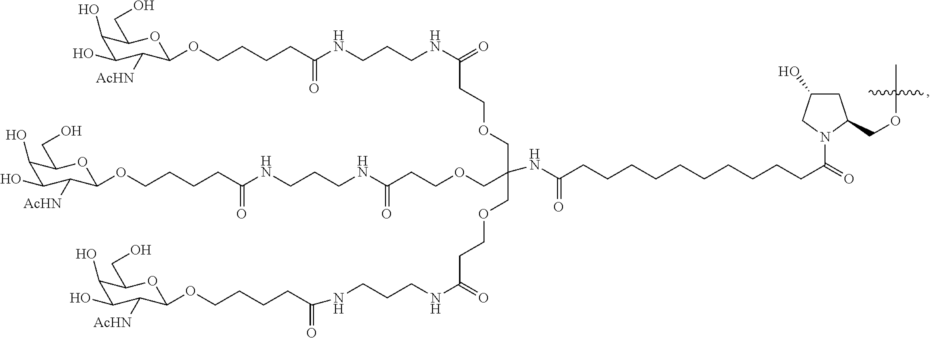 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

wherein the dsRNA agent is in a free acid form.

In another aspect, the present invention provides a pharmaceutical composition for inhibiting expression of a Serpinc1 gene, comprising a double-stranded ribonucleic acid (dsRNA) agent at a concentration of about 106 mg/mL and phosphate buffered saline (PBS) at a concentration of about 5 mM, wherein the pH of the pharmaceutical composition is about 6.8 to about 7.2, wherein the osmolality of the pharmaceutical composition is about 300 mOsm/kg, wherein the dsRNA agent has a sense strand consisting of the nucleotide sequence of 5′-GfsgsUfuAfaCfaCfCfAfuUfuAfcUfuCfaAf-3′ (SEQ ID NO:941) and an antisense strand consisting of the nucleotide sequence of 5′-usUfsgAfaGfuAfaAfuggUfgUfuAfaCfcsasg-3′ (SEQ ID NO:960), wherein a, g, c, and u are 2′-O-methyl (2′-OMe) A, G, C, and U; Af, Gf, Cf, and Uf are 2′-fluoro A, G, C, U; and s is a phosphorothioate linkage, and wherein a ligand is conjugated to the 3′ end of the sense strand via a linker, and wherein the ligand and the linker have the following structure:

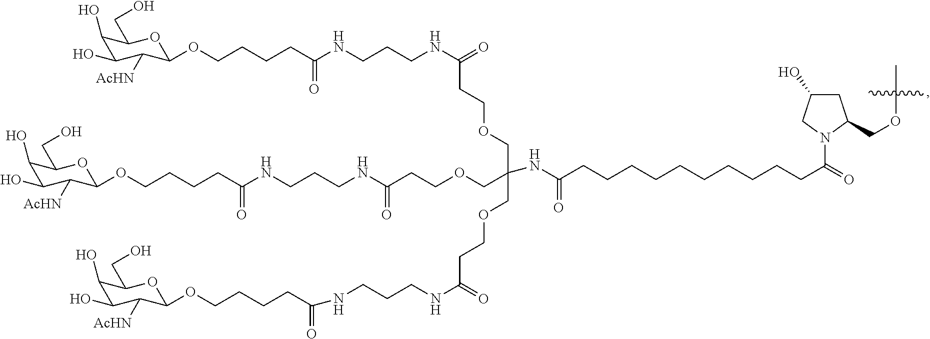 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

wherein the dsRNA agent is in a salt form.

In one embodiment, the salt form is a sodium salt form.

In one embodiment, substantially all of the phosphodiester and/or phosphorothiotate groups in the agent comprise a sodium counterion. In another embodiment, all of the phosphodiester and/or phosphorothiotate groups in the agent comprise a sodium counterion.

In one embodiment, the composition is stable for between about 6 months to about 36 months when stored at about 2° C. to about 8° C. In another embodiment, the composition is stable for between about 6 months to about 36 months when stored at about 25° C. and 60% relative humidity (RH). In yet another embodiment, the composition is stable for about 6 months when stored at about 40° C. and 75% relative humidity (RH).

In one embodiment, the composition is stable up to about 36 months when stored at about 2° C. to about 8° C. In another embodiment, the composition is stable up to about 36 months when stored at about 25° C. and 60% relative humidity (RH). In yet another embodiment, the composition is stable for up to 6 months when stored at about 40° C. and 75% relative humidity (RH).

In one embodiment, the composition comprises not less than (NLT) about 95.0 area % duplex and not more than (NMT) about 5 area % total impurities of duplex as determined by purity non-denaturing IPRP-HPLC.

In one embodiment, the composition comprises not less than (NLT) about 85.0 area % total single strands as determined by purity denaturing AX-HPLC.

In one embodiment, the composition comprises not less than (NLT) about 80.0 area % total single strands as determined by purity denaturing IPRP-HPLC.

The present invention also provides a vial comprising the foregoing pharmaceutical compositions. The vials may include about 0.5 mL to about 2.0 ml of the pharmaceutical composition; or about 0.8 ml of the pharmaceutical composition.

The present invention further provides a syringe comprising the foregoing pharmaceutical compositions.

The syringes of the invention may be a 1 ml syringe; or a 3 ml syringe. In one embodiment, the syringe is a 1 ml single-use syringe.

The syringes of the invention may include a 29 G needle; or a 30 G needle. In one embodiment, the needle is a 29 G needle.

In one embodiment, the syringe is a pre-filled syringe.

In another aspect, the present invention provides a 2 ml vial comprising about 0.8 ml of a pharmaceutical composition for inhibiting expression of a Serpinc1 gene, wherein the pharmaceutical composition comprises a double-stranded ribonucleic acid (dsRNA) agent at a concentration of about 106 mg/mL and phosphate buffered saline (PBS) at a concentration of about 5 mM, wherein the pH of the pharmaceutical composition is about 6.8 to about 7.2, wherein the osmolality of the pharmaceutical composition is about 300 mOsm/kg, wherein the dsRNA agent has a sense strand consisting of the nucleotide sequence of 5′-GfsgsUfuAfaCfaCfCfAfuUfuAfcUfuCfaAf-3′ (SEQ ID NO:941) and an antisense strand consisting of the nucleotide sequence of 5′-usUfsgAfaGfuAfaAfuggUfgUfuAfaCfcsasg-3′ (SEQ ID NO:960), wherein a, g, c, and u are 2′-O-methyl (2′-OMe) A, G, C, and U; Af, Gf, Cf, and Uf are 2′-fluoro A, G, C, U; and s is a phosphorothioate linkage, and wherein a ligand is conjugated to the 3′ end of the sense strand via a linker, and wherein the ligand and the linker have the following structure:

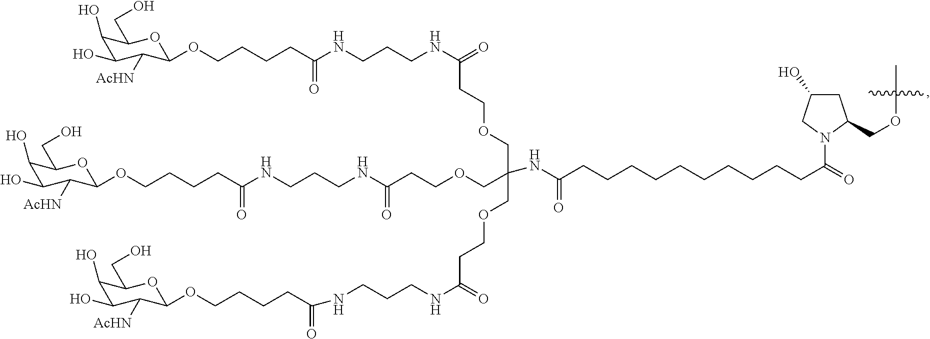 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

wherein the dsRNA agent is in a sodium salt form and all of the phosphodiester and/or phosphorothioate groups in the agent comprise a sodium counterion.

In another aspect, the present invention provides a 1 ml pre-filled single-use syringe comprising a 29 G needle, wherein the syringe comprises about 0.8 ml of a pharmaceutical composition for inhibiting expression of a Serpinc1 gene, wherein the pharmaceutical composition comprises a double-stranded ribonucleic acid (dsRNA) agent at a concentration of about 106 mg/mL and phosphate buffered saline (PBS) at a concentration of about 5 mM, wherein the pH of the pharmaceutical composition is about 6.8 to about 7.2, wherein the osmolality of the pharmaceutical composition is about 300 mOsm/kg, wherein the dsRNA agent has a sense strand consisting of the nucleotide sequence of 5′-GfsgsUfuAfaCfaCfCfAfuUfuAfcUfuCfaAf-3′ (SEQ ID NO:941) and an antisense strand consisting of the nucleotide sequence of 5′-usUfsgAfaGfuAfaAfuggUfgUfuAfaCfcsasg-3′ (SEQ ID NO:960), wherein a, g, c, and u are 2′-O-methyl (2′-OMe) A, G, C, and U; Af, Gf, Cf, and Uf are 2′-fluoro A, G, C, U; and s is a phosphorothioate linkage, and wherein a ligand is conjugated to the 3′ end of the sense strand via a linker, and wherein the ligand and the linker have the following structure:

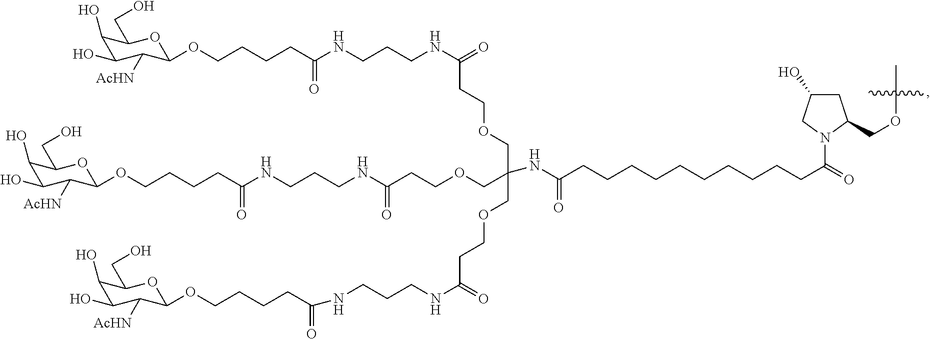 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

wherein the dsRNA agent is in a sodium salt form and all of the phosphodiester and/or phosphorothioate groups in the agent comprise a sodium counterion.

In another aspect, the present invention provides a 2 ml vial comprising about 0.8 ml of a pharmaceutical composition for inhibiting expression of a Serpinc1 gene, wherein the pharmaceutical composition comprises a double-stranded ribonucleic acid (dsRNA) agent at a concentration of about 106 mg/mL and phosphate buffered saline (PBS) at a concentration of about 5 mM, wherein the pH of the pharmaceutical composition is about 6.8 to about 7.2, wherein the osmolality of the pharmaceutical composition is about 300 mOsm/kg, wherein the dsRNA agent has the structure

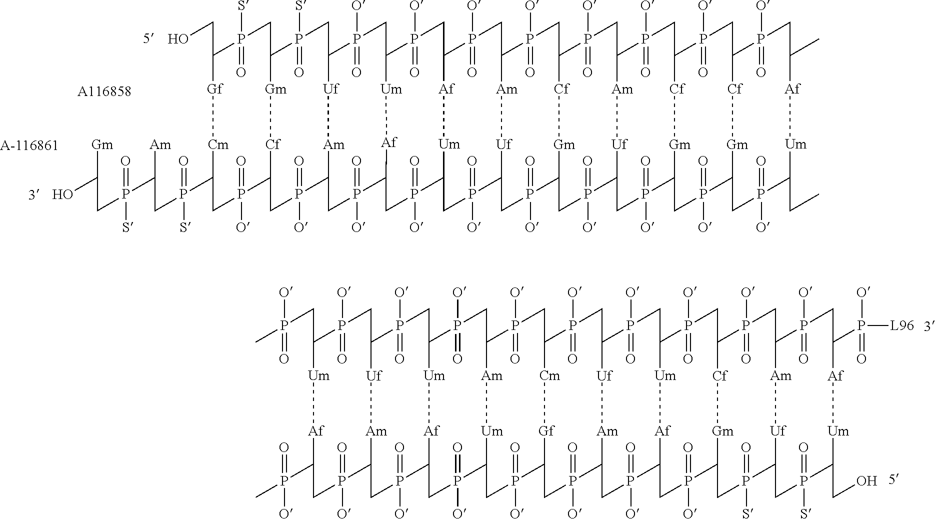

wherein Am, Gm, Cm, and Um are 2′-O-methyl (2′ OMe) A, G, C, and U; Af, Gf, Cf, and Uf are 2′-fluoro A, G, C, U; s is a phosphorothioate linkage; and wherein L96 is a ligand and linker having the following structure:

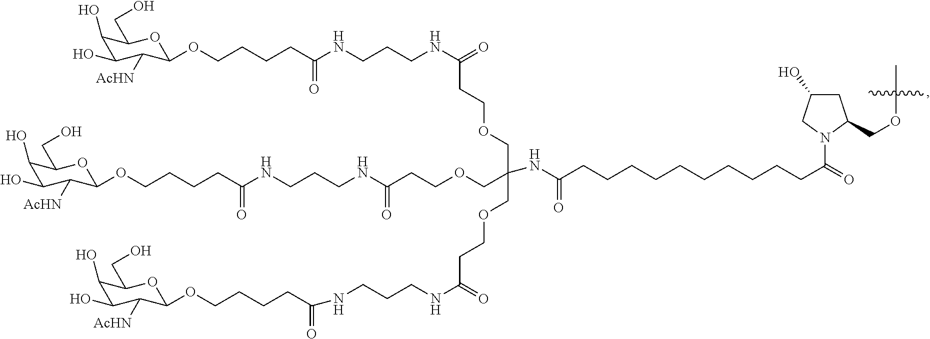 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

wherein the dsRNA agent is in a sodium salt form and all of the phosphodiester and/or phosphorothioate groups in the agent comprise a sodium counterion.

In another aspect, the present invention provides a 1 ml pre-filled single-use syringe comprising a 29 G needle, wherein the syringe comprises about 0.8 ml of a pharmaceutical composition for inhibiting expression of a Serpinc1 gene, wherein the pharmaceutical composition comprises a double-stranded ribonucleic acid (dsRNA) agent at a concentration of about 106 mg/mL and phosphate buffered saline (PBS) at a concentration of about 5 mM, wherein the pH of the pharmaceutical composition is about 6.8 to about 7.2, wherein the osmolality of the pharmaceutical composition is about 300 mOsm/kg, wherein the dsRNA agent has the structure

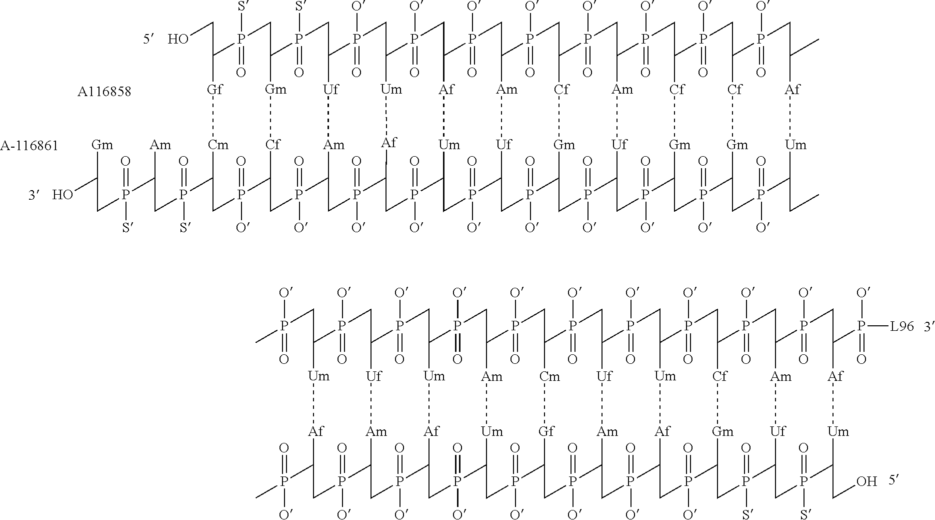

wherein Am, Gm, Cm, and Um are 2′-O-methyl (2′-OMe) A, G, C, and U; Af, Gf, Cf, and Uf are 2′-fluoro A, G, C, U; s is a phosphorothioate linkage; and wherein L96 is a ligand and linker having the following structure:

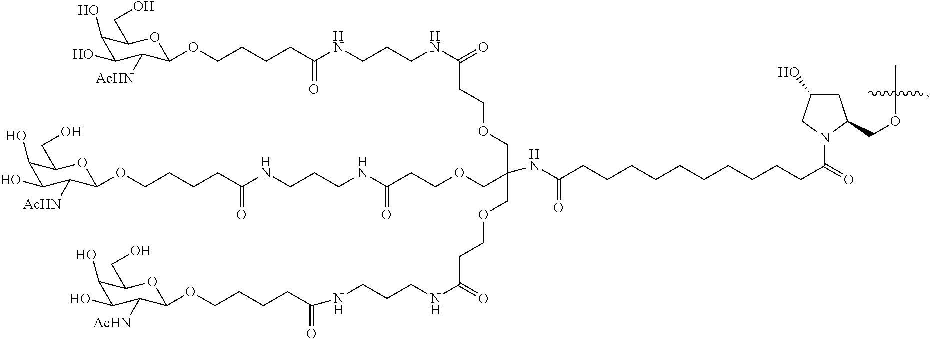 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

wherein the dsRNA agent is in a sodium salt form and all of the phosphodiester and/or phosphorothioate groups in the agent comprise a sodium counterion.

In another aspect, the present invention provides a 2 ml vial comprising about 0.8 ml of a pharmaceutical composition for inhibiting expression of a Serpinc1 gene, wherein the pharmaceutical composition comprises a double-stranded ribonucleic acid (dsRNA) agent at a concentration of about 106 mg/mL and phosphate buffered saline (PBS) at a concentration of about 5 mM, wherein the pH of the pharmaceutical composition is about 6.8 to about 7.2, wherein the dsRNA agent has a sense strand consisting of the nucleotide sequence of 5′-GfsgsUfuAfaCfaCfCfAfuUfuAfcUfuCfaAf-3′ (SEQ ID NO:941) and an antisense strand consisting of the nucleotide sequence of 5′-usUfsgAfaGfuAfaAfuggUfgUfuAfaCfcsasg-3′ (SEQ ID NO:960), wherein a, g, c, and u are 2′-O-methyl (2′-OMe) A, G, C, and U; Af, Gf, Cf, and Uf are 2′-fluoro A, G, C, U; and s is a phosphorothioate linkage, and wherein a ligand is conjugated to the 3′ end of the sense strand via a linker, and wherein the ligand and the linker have the following structure:

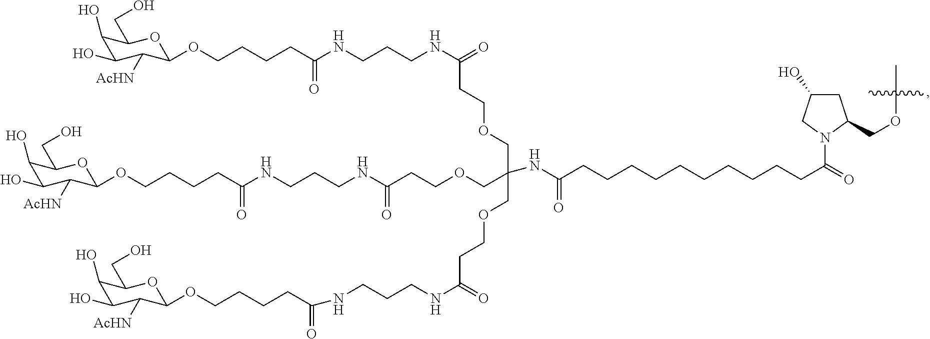 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

wherein the dsRNA agent is in a sodium salt form and all of the phosphodiester and/or phosphorothioate groups in the agent comprise a sodium counterion.

In another aspect, the present invention provides a 1 ml pre-filled single-use syringe comprising a 29 G needle, wherein the syringe comprises about 0.8 ml of a pharmaceutical composition for inhibiting expression of a Serpinc1 gene, wherein the pharmaceutical composition comprises a double-stranded ribonucleic acid (dsRNA) agent at a concentration of about 106 mg/mL and phosphate buffered saline (PBS) at a concentration of about 5 mM, wherein the pH of the pharmaceutical composition is about 6.8 to about 7.2, wherein the dsRNA agent has a sense strand consisting of the nucleotide sequence of 5′-GfsgsUfuAfaCfaCfCfAfuUfuAfcUfuCfaAf-3′ (SEQ ID NO:941) and an antisense strand consisting of the nucleotide sequence of 5′-usUfsgAfaGfuAfaAfuggUfgUfuAfaCfcsasg-3′ (SEQ ID NO:960), wherein a, g, c, and u are 2′-O-methyl (2′-OMe) A, G, C, and U; Af, Gf, Cf, and Uf are 2′-fluoro A, G, C, U; and s is a phosphorothioate linkage, and wherein a ligand is conjugated to the 3′ end of the sense strand via a linker, and wherein the ligand and the linker have the following structure:

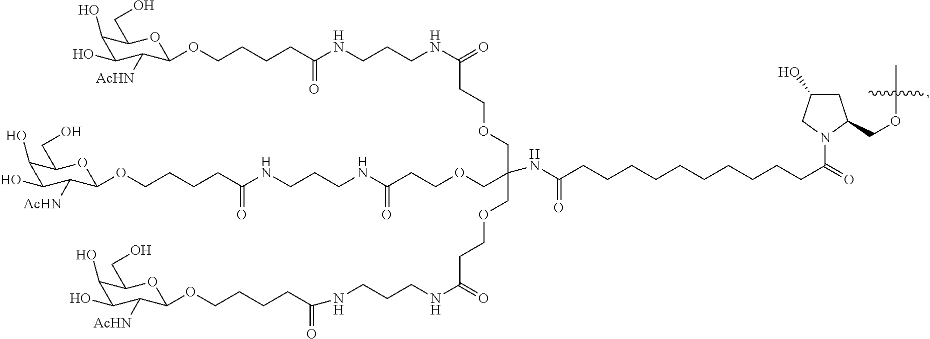 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

wherein the dsRNA agent is in a sodium salt form and all of the phosphodiester and/or phosphorothioate groups in the agent comprise a sodium counterion.

In another aspect, the present invention provides a 2 ml vial comprising about 0.8 ml of a pharmaceutical composition for inhibiting expression of a Serpinc1 gene, wherein the pharmaceutical composition comprises a double-stranded ribonucleic acid (dsRNA) agent at a concentration of about 106 mg/mL and phosphate buffered saline (PBS) at a concentration of about 5 mM, wherein the pH of the pharmaceutical composition is about 6.8 to about 7.2, wherein the dsRNA agent has the structure

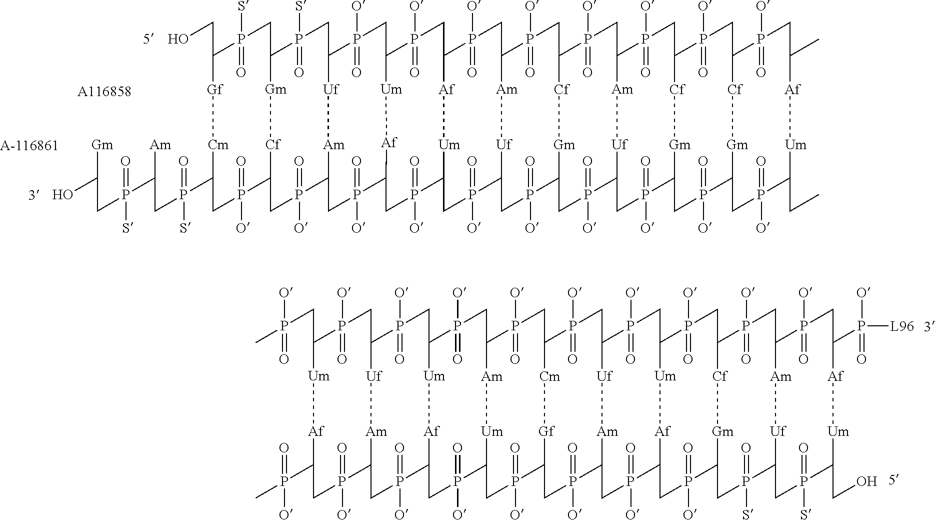

wherein Am, Gm, Cm, and Um are 2′-O-methyl (2′ OMe) A, G, C, and U; Af, Gf, Cf, and Uf are 2′-fluoro A, G, C, U; s is a phosphorothioate linkage; and wherein L96 is a ligand and linker having the following structure:

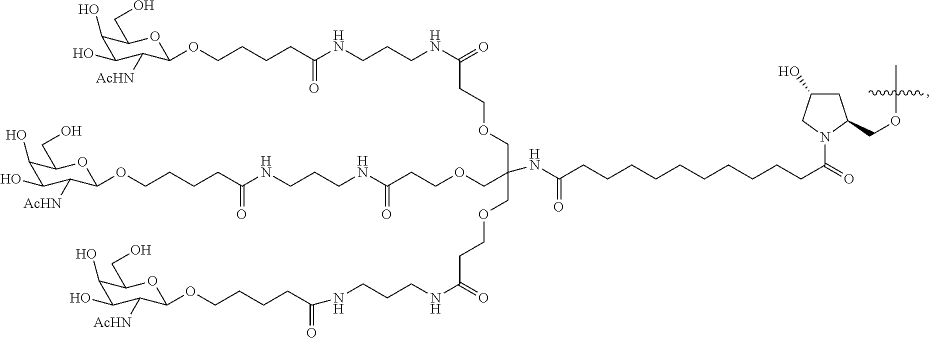 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

wherein the dsRNA agent is in a sodium salt form and all of the phosphodiester and/or phosphorothioate groups in the agent comprise a sodium counterion.

In another aspect, the present invention provides a 1 ml pre-filled single-use syringe comprising a 29 G needle, wherein the syringe comprises about 0.8 ml of a pharmaceutical composition for inhibiting expression of a Serpinc1 gene, wherein the pharmaceutical composition comprises a double-stranded ribonucleic acid (dsRNA) agent at a concentration of about 106 mg/mL and phosphate buffered saline (PBS) at a concentration of about 5 mM, wherein the pH of the pharmaceutical composition is about 6.8 to about 7.2, wherein the dsRNA agent has the structure

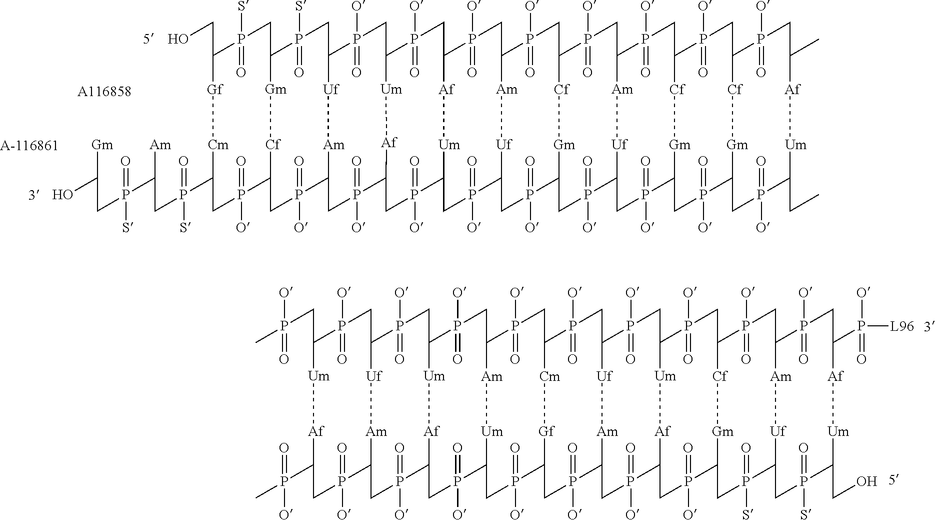

wherein Am, Gm, Cm, and Um are 2′-O-methyl (2′-OMe) A, G, C, and U; Af, Gf, Cf, and Uf are 2′-fluoro A, G, C, U; s is a phosphorothioate linkage; and wherein L96 is a ligand and linker having the following structure:

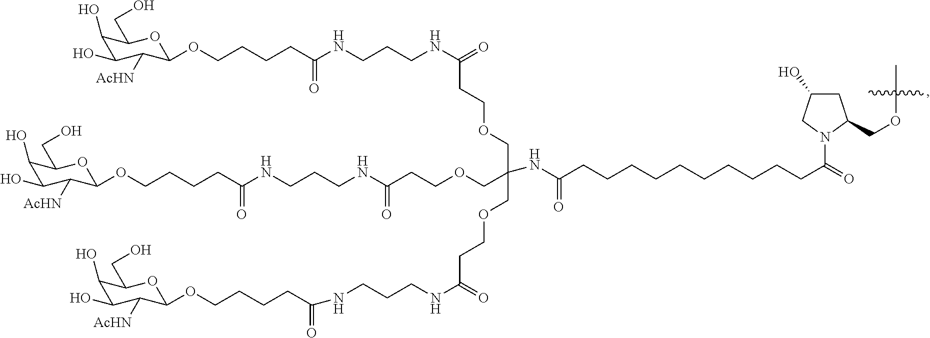 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

wherein the dsRNA agent is in a sodium salt form and all of the phosphodiester and/or phosphorothioate groups in the agent comprise a sodium counterion.

### BRIEF SUMMARY OF THE DRAWINGS

FIG. 1 depicts a representative non-denaturing Ion-Pair Reversed-Phase High Performance Liquid Chromatography (IP RP-HPLC) chromatogram of fitusiran drug product. Occasional slight splitting of the fitusiran duplex peak was observed due to partial resolution of different stereoisomers of phosphorothioates in the siRNA duplex. Mass spectrometric analysis of the fitusiran peak confirmed the presence of the expected masses of both single strands in equal ratio throughout the entire duplex peak.

FIG. 2 depicts a representative denaturing Anion Exchange High Performance Liquid Chromatography (AX-HPLC) chromatogram of single strands in duplex in the fitusiran drug product.

FIG. 3 depicts a representative denaturing Ion-Pair Reversed-Phase High Performance Liquid Chromatography (IP RP-HPLC) chromatographic profile of single strands in duplex in the fitusiran drug product.

### DETAILED DESCRIPTION OF THE INVENTION

The present invention provides pharmaceutical compositions comprising an iRNA agent which effects the RNA-induced silencing complex (RISC)-mediated cleavage of RNA transcripts of a Serpinc1 gene. The present invention is based, at least in part, on the discovery of stable pharmaceutical compostions comprising such agents which have improved satability, efficacy, durability, and ease of administration as compared to other compositions comprising a dsRNA agent that inhibits the expression of a Serpinc1 gene. Such pharmaceutical compositions are useful for inhibiting the expression of a Serpinc1 gene and/or for treating a subject having a disorder that would benefit from inhibiting or reducing the expression of a Serpinc1 gene, e.g., a bleeding disorder, such as a hemophilia.

The following detailed description discloses how to make and use compositions containing iRNAs to inhibit the expression of a Serpinc1 gene, as well as compositions, uses, and methods for treating subjects having diseases and disorders that would benefit from inhibition and/or reduction of the expression of this gene.

### I. Definitions

In order that the present invention may be more readily understood, certain terms are first defined. In addition, it should be noted that whenever a value or range of values of a parameter are recited, it is intended that values and ranges intermediate to the recited values are also intended to be part of this invention.

The articles “a” and “an” are used herein to refer to one or to more than one (i.e., to at least one) of the grammatical object of the article. By way of example, “an element” means one element or more than one element, e.g., a plurality of elements.

The term “about” is used herein to mean within the typical ranges of tolerances in the art. For example, “about” can be understood as about 2 standard deviations from the mean. In certain embodiments, about means +10%. In certain embodiments, about means +5%. When about is present before a series of numbers or a range, it is understood that “about” can modify each of the numbers in the series or range.

The term “including” is used herein to mean, and is used interchangeably with, the phrase “including but not limited to”.

The term “or” is used herein to mean, and is used interchangeably with, the term “and/or,” unless context clearly indicates otherwise.

The term “pharmaceutical composition,” as used herein, refers to a composition that it is useful for treating a disease or disorder in a subject, e.g., a human subject.

The term “pharmaceutical administration” refers to the delivery of a composition comprising a dsRNA agent, as described herein, to a subject for treating a disease or disorder. Thus, “suitable for pharmaceutical administration” such as “suitable for subcutaneous administration” describes a composition comprising a dsRNA agent which may be used to treat a disease or disorder in a subject bu subcutaneous administration of the pharmaceutical composition. A pharmaceutical composition is suitable for pharmaceutical administration, e.g., suitable for subcutaneous administration.

The term “osmolality” refers to the number of osmoles of solute per kilogram of solvent. It is expressed in terms of osmol/kg or Osm/kg. An “osmole” is a unit of measurement that describes the number of moles of a compound that contribute to the osmotic pressure of a chemical solution.

As used herein, “Serpinc1” refers to a particular polypeptide expressed in a cell. Serpinc1 is also known as serpin peptidase inhibitor, clade C (antithrombin; AT), member 1; antithrombin III; AT3; antithrombin; and heparin cofactor 1. The sequence of a human Serpinc1 mRNA transcript can be found at, for example, GenBank Accession No. GI:254588059 (NM_000488; SEQ ID NO:1). The sequence of rhesus Serpinc1 mRNA can be found at, for example, GenBank Accession No. GI:157167169 (NM_001104583; SEQ ID NO:2). The sequence of mouse Serpinc1 mRNA can be found at, for example, GenBank Accession No. GI:237874216 (NM_080844; SEQ ID NO:3). The sequence of rat Serpinc1 mRNA can be found at, for example, GenBank Accession No. GI:58865629 (NM_001012027; SEQ ID NO:4).

The term “Serpinc1” as used herein also refers to a particular polypeptide expressed in a cell by naturally occurring DNA sequence variations of the Serpinc1 gene, such as a single nucleotide polymorphism in the Serpinc1 gene. Numerous SNPs within the Serpinc1 gene have been identified and may be found at, for example, NCBI dbSNP (see, e.g., www.ncbi.nlm.nih.gov/snp). Non-limiting examples of SNPs within the Serpinc1 gene may be found at, NCBI dbSNP Accession Nos. rs677; rs5877; rs5878; rs5879; rs941988; rs941989; rs1799876; rs19637711; rs2008946; and rs2227586.

As used herein, a “subject” is an animal, such as a mammal, including a primate (such as a human, a non-human primate, e.g., a monkey, and a chimpanzee), a non-primate (such as a cow, a pig, a camel, a llama, a horse, a goat, a rabbit, a sheep, a hamster, a guinea pig, a cat, a dog, a rat, a mouse, a horse, and a whale), or a bird (e.g., a duck or a goose). In one embodiment, the subject is a human, such as a human being treated or assessed for a disease, disorder or condition that would benefit from reduction in Serpinc1 expression; a human at risk for a disease, disorder or condition that would benefit from reduction in Serpinc1 expression; a human having a disease, disorder or condition that would benefit from reduction in Serpinc1 expression; and/or human being treated for a disease, disorder or condition that would benefit from reduction in Serpinc1 expression as described herein.

The term “inhibiting,” as used herein, is used interchangeably with “reducing,” “silencing,” “downregulating,” “suppressing” and other similar terms, and includes any level of inhibition.

The phrase “inhibiting expression of a Serpinc1,” as used herein, includes inhibition of expression of any Serpinc1 gene (such as, e.g., a mouse Serpinc1 gene, a rat Serpinc1 gene, a monkey Serpinc1 gene, or a human Serpinc1 gene) as well as variants or mutants of a Serpinc1 gene that encode a Serpinc1 protein.

“Inhibiting expression of a Serpinc1 gene” includes any level of inhibition of a Serpinc1 gene, e.g., at least partial suppression of the expression of a Serpinc1 gene, such as an inhibition by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%.

The expression of a Serpinc1 gene may be assessed based on the level of any variable associated with Serpinc1 gene expression, e.g., Serpinc1 mRNA level, Serpinc1 protein level, or, for example, thrombin:antithrombin complex levels as a measure of thrombin generation portential, bleeding time, prothrombin time (PT), platelet count, and/or activated partial thromboplastin time (aPTT). Inhibition may be assessed by a decrease in an absolute or relative level of one or more of these variables compared with a control level. The control level may be any type of control level that is utilized in the art, e.g., a pre-dose baseline level, or a level determined from a similar subject, cell, or sample that is untreated or treated with a control (such as, e.g., buffer only control or inactive agent control).

In one embodiment, at least partial suppression of the expression of a Serpinc1 gene, is assessed by a reduction of the amount of Serpinc1 mRNA which can be isolated from or detected in a first cell or group of cells in which a Serpinc1 gene is transcribed and which has or have been treated such that the expression of a Serpinc1 gene is inhibited, as compared to a second cell or group of cells substantially identical to the first cell or group of cells but which has or have not been so treated (control cells). The degree of inhibition may be expressed in terms of

$$\frac{( mRNA in control cells ) - ( mRNA in treated cells )}{( mRNA in control cells )} \cdot 100 \%$$

The phrase “contacting a cell with an RNAi agent,” such as a dsRNA, as used herein, includes contacting a cell by any possible means. Contacting a cell with an RNAi agent includes contacting a cell in vitro with the iRNA or contacting a cell in vivo with the iRNA. The contacting may be done directly or indirectly. Thus, for example, the RNAi agent may be put into physical contact with the cell by the individual performing the method, or alternatively, the RNAi agent may be put into a situation that will permit or cause it to subsequently come into contact with the cell.

Contacting a cell in vitro may be done, for example, by incubating the cell with the RNAi agent. Contacting a cell in vivo may be done, for example, by injecting the RNAi agent into or near the tissue where the cell is located, or by injecting the RNAi agent into another area, e.g., the bloodstream or the subcutaneous space, such that the agent will subsequently reach the tissue where the cell to be contacted is located. For example, the RNAi agent may contain and/or be coupled to a ligand, e.g., GalNAc3, that directs the RNAi agent to a site of interest, e.g., the liver. Combinations of in vitro and in vivo methods of contacting are also possible. For example, a cell may also be contacted in vitro with an RNAi agent and subsequently transplanted into a subject.

### II. Pharmaceutical Compositions of the Invention

The present invention provides stable pharmaceutical compositions comprising a double-stranded ribonucleic acid (dsRNA) agent that inhibits expression of a Serpinc1 gene. The pharmaceutical compositions of the invention include a dsRNA agent, as described herein, and phosphate buffered saline (PBS), and are suitable for subcutaneous administration to a subject. The pharmaceutical compositions containing the dsRNA agents are useful for treating a disease or disorder associated with the expression or activity of a Serpinc1 gene, e.g. a Serpinc1-associated disease, e.g., a hemophilia. The pharmaceutical compositions of the invention may be administered in dosages sufficient to inhibit expression of a Serpinc1 gene.

In one embodiment, the pharmaceutical compositions of the invention include dsRNA agents of the invention in a free acid form. In another embodiment, the pharmaceutical compositions of the invention include dsRNA agents of the invention in a sodium salt form. In certain embodiments, when the dsRNA agents of the invention are in the sodium salt form, sodium ions are present in the agent as counterions (in order to maintain electric neutrality), for substantially all of the phosphodiester and/or phosphorothiotate groups present in the agent. Agents in which substantially all of the phosphodiester and/or phosphorothioate linkages have a sodium counterion include not more than 5, 4, 3, 2, or 1 phosphodiester and/or phosphorothioate linkages without a sodium counterion. In some embodiments, when the dsRNA agents of the invention are in the sodium salt form, sodium ions are present in the agent as counterions for all of the phosphodiester and/or phosphorothiotate groups present in the agent.

The pharmaceutical compositions of the invention may include a dsRNA agent at a concentration of about 50 mg/mL to about 200 mg/mL, about 50 mg/mL to about 150 mg/mL; about 90 mg/mL to about 110 mg/mL, about 90 mg/mL to about 100 mg/mL, or about 80 mg/mL to about 110 mg/mL, e.g., about 50 mg/mL, 55 mg/mL, 60 mg/mL, 65 mg/mL, 70 mg/mL, 75 mg/mL, 80 mg/mL, 85 mg/mL, 90 mg/mL, 95 mg/mL, 100 mg/mL, 105 mg/mL, 106 mg/mL, 110 mg/mL, 115 mg/mL, 120 mg/mL, 125 mg/mL, 130 mg/mL, 135 mg/mL, 140 mg/mL, 145 mg/mL, 150 mg/mL, 155 mg/mL, 160 mg/mL, 165 mg/mL, 170 mg/mL, 175 mg/mL, 180 mg/mL, 185 mg/mL, 190 mg/mL, 195 mg/mL, or about 200 mg/mL. In one embodiment, the pharmaceutical compositions of the invention include a dsRNA agent at a concentration of about 100 mg/mL. Values intermediate to the above recited ranges and values are also intended to be part of this invention. In addition, ranges of values using a combination of any of the above recited values as upper and/or lower limits are intended to be included.

The pharmaceutical compositions of the invention may include PBS. In one embodiment, the PBS includes sodium chloride and sodium phosphate, but does not include potassium chloride and/or potassium phosphate. In another embodiment, the PBS includes sodium chloride, sodium phosphate, and potassium chloride. In yet another embodiment, the PBS includes sodium chloride, sodium phosphate, and potassium phosphate. In one embodiment, the PBS includes sodium chloride, sodium phosphate, potassium chloride, and potassium phosphate. In certain embodiments, e.g., when the PBS includes sodium chloride and sodium phosphate, the PBS may be at a concentration of about 1 mM to about 10 mM; or about 3 mM to about 6 mM, e.g., about 1 mM, 1.5 mM, 2 mM, 2.5 mM, 3 mM, 3.5 mM, 4 mM, 4.5 mM, 5 mM, 6.5 mM, 7 mM, 7.5. mM, 9 mM, 8.5 mM, 9 mM, 9.5 mM, or about 10 mM PBS. In one embodiment of the invention, a pharmaceutical composition of the invention PBS at a concentration of about 5 mM (e.g., about 0.64 mM NaH₂PO₄, about 4.36 mM Na₂HPO₄, about 85 mM NaCl). Values intermediate to the above recited ranges and values are also intended to be part of this invention. In addition, ranges of values using a combination of any of the above recited values as upper and/or lower limits are intended to be included.

In one embodiment, the pharmaceutical compositions of the invention are preservative-free. In another embodiment of the invention, the pharmaceutical compositions of the invention include a preservative.

The pH of the pharmaceutical compositions of the invention are suitable for subcutaneous administration and may be between about 5.0 to about 8.0, about 5.5 to about 8.0, about 6.0 to about 8.0, about 6.5 to about 8.0, about 7.0 to about 8.0, about 5.0 to about 7.5, about 5.5 to about 7.5, about 6.0 to about 7.5, about 6.5 to about 7.5, about 5.0 to about 7.2, about 5.25 to about 7.2, about 5.5 to about 7.2, about 5.75 to about 7.2, about 6.0 to about 7.2, about 6.5 to about 7.2, or about 6.8 to about 7.2. Ranges and values intermediate to the above recited ranges and values are also intended to be part of this invention.

The osmolality of the pharmaceutical compositions of the invention may be suitable for subcutaneous administration, such as no more than about 400 mOsm/kg, e.g., between 50 and 400 mOsm/kg, between 75 and 400 mOsm/kg, between 100 and 400 mOsm/kg, between 125 and 400 mOsm/kg, between 150 and 400 mOsm/kg, between 175 and 400 mOsm/kg, between 200 and 400 mOsm/kg, between 250 and 400 mOsm/kg, between 300 and 400 mOsm/kg, between 50 and 375 mOsm/kg, between 75 and 375 mOsm/kg, between 100 and 375 mOsm/kg, between 125 and 375 mOsm/kg, between 150 and 375 mOsm/kg, between 175 and 375 mOsm/kg, between 200 and 375 mOsm/kg, between 250 and 375 mOsm/kg, between 300 and 375 mOsm/kg, between 50 and 350 mOsm/kg, between 75 and 350 mOsm/kg, between 100 and 350 mOsm/kg, between 125 and 350 mOsm/kg, between 150 and 350 mOsm/kg, between 175 and 350 mOsm/kg, between 200 and 350 mOsm/kg, between 250 and 350 mOsm/kg, between 50 and 325 mOsm/kg, between 75 and 325 mOsm/kg, between 100 and 325 mOsm/kg, between 125 and 325 mOsm/kg, between 150 and 325 mOsm/kg, between 175 and 325 mOsm/kg, between 200 and 325 mOsm/kg, between 250 and 325 mOsm/kg, between 300 and 325 mOsm/kg, between 300 and 350 mOsm/kg, between 50 and 300 mOsm/kg, between 75 and 300 mOsm/kg, between 100 and 300 mOsm/kg, between 125 and 300 mOsm/kg, between 150 and 300 mOsm/kg, between 175 and 300 mOsm/kg, between 200 and 300 mOsm/kg, between 250 and 300, between 50 and 250 mOsm/kg, between 75 and 250 mOsm/kg, between 100 and 250 mOsm/kg, between 125 and 250 mOsm/kg, between 150 and 250 mOsm/kg, between 175 and 350 mOsm/kg, between 200 and 250 mOsm/kg, e.g., about 50, 55, 60, 65, 70, 75, 80, 85, 90, 95, 100, 105, 110, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 295, 300, 305, 310, 320, 325, 330, 335, 340, 345, 350, 355, 360, 365, 370, 375, 380, 385, 390, 395, or about 400 mOsm/kg. Ranges and values intermediate to the above recited ranges and values are also intended to be part of this invention. In addition, ranges of values using a combination of any of the above recited values as upper and/or lower limits are intended to be included.

The pharmaceutical compositions of the invention are physically and chemically stable.

As used herein, the term “stable” refers to a pharmaceutical composition and/or a dsRNA agent within such a pharmaceutical composition which essentially retains its physical stability and/or chemical stability and/or biological activity. Various analytical techniques for measuring stability of the composition and the dsRNA agent therein are available in the art and are described herein.

A pharmaceutical composition (or dsRNA agent within such a composition) “retains its physical stability” if it shows substantially no signs of, e.g., increased impurities upon visual examination or UV examination of color and/or clarity, or as measured by, for example HPLC analysis, e.g., denaturing IP RP-HPLC, non-denaturing IP RP-HPLC, and/or denaturing AX-HPLC analysis.

A dsRNA agent “retains its chemical stability” in pharmaceutical composition, if the chemical stability at a given time is such that the dsRNA agent is considered to still retain its biological activity. Chemical stability can be assessed by, e.g., detecting and/or quantifying chemically altered forms of the dsRNA duplex and/or chemically altered forms of the sense strand and/or antisense strand. Chemical alteration may involve size modification and/or sodium content change which can be evaluated by, for example duplex retention time and/or identification of the molecular weight of the single strands forming the duplex using, e.g., non-denaturing IP RP-HPLC, identification by melting temperature using, e.g., thermal UV spectrophotometry, and/or by sodium content (on an anhydrous basis) using, for example, Flame Atomic Absorption (flame AAS)/inductively coupled plasma optical emission spectrometry (ICP-OES).

A dsRNA agent “retains its biological activity” in a pharmaceutical composition, if the dsRNA agent in a composition is biologically active for its intended purpose. For example, biological activity is retained if the biological activity of an dsRNA agent in the composition is within about 30%, about 20%, or about 10% (within the errors of the assay) of the biological activity exhibited at the time the composition was prepared (e.g., as determined by an in vitro RT-PCR assay).

For example, in some embodiments, the compositions of the invention are stable for between about 6 months to about 36 months when stored at about 2° C. to about 8° C. In other embodiments, the compositions of the invention are stable for between about 6 months to about 36 months when stored at about 25° C. and 60% relative humidity (RH). In still other embodiment, the compositions of the invention are stable for about 6 months when stored at about 40° C. and 75% relative humidity (RH).

In some embodiments, the compositions of the invention are stable for up to about 36 months when stored at about 2° C. to about 8° C. In other embodiments, the compositions of the invention are stable for up to about 36 months when stored at about 25° C. and 60% relative humidity (RH). In still other embodiment, the compositions of the invention are stable for up to 6 months when stored at about 40° C. and 75% relative humidity (RH).

In one embodiment, the compositions of the invention comprise not less than (NLT) about 95.0 area % duplex and not more than (NMT) about 5 area % total impurities of duplex as determined by purity non-denaturing IPRP-HPLC. In another embodiment, the pharmaceutical compositions of the invention comprise not less than (NLT) about 85.0 area % total single strands as determined by purity denaturing AX-HPLC. In yet another embodiment, the pharmaceutical compositions of the invention comprise not less than (NLT) about 80.0 area % total single strands as determined by purity denaturing IPRP-HPLC.

In one aspect, the present invention provides a pharmaceutical composition for inhibiting expression of a Serpinc1 gene. The pharmaceutical composition includes a double-stranded ribonucleic acid (dsRNA) agent at a concentration of about 50 mg/mL to about 200 mg/mL and phosphate buffered saline (PBS) at a concentration of about 1 mM to about 10 mM, wherein the pH and the osmolality of the pharmaceutical composition are suitable for subcutaneous administration to a subject, wherein the dsRNA agent comprises a sense strand and an antisense strand, the antisense strand comprising a region of complementarity to an mRNA encoding Serpinc1 which comprises at least 15 contiguous nucleotides differing by no more than 3 nucleotides from the nucleotide sequence of 5′-UUGAAGUAAAUGGUGUUAACCAG-3′ (SEQ ID NO: 15), wherein substantially all of the nucleotides of the sense strand and substantially all of the nucleotides of the antisense strand are modified nucleotides, wherein the sense strand is conjugated to a ligand attached at the 3′-terminus, and wherein the dsRNA agent is in a free acid form.

In another aspect, the present invention provides a pharmaceutical composition for inhibiting expression of a Serpinc1 gene. The pharmaceutical composition includes a double-stranded ribonucleic acid (dsRNA) agent at a concentration of about 50 mg/mL to about 200 mg/mL and phosphate buffered saline (PBS) at a concentration of about 1 mM to about 10 mM, wherein the pH and the osmolality of the pharmaceutical composition are suitable for subcutaneous administration to a subject, wherein the dsRNA agent comprises a sense strand and an antisense strand, the antisense strand comprising a region of complementarity to an mRNA encoding Serpinc1 which comprises at least 15 contiguous nucleotides differing by no more than 3 nucleotides from the nucleotide sequence of 5′-UUGAAGUAAAUGGUGUUAACCAG-3′ (SEQ ID NO: 15), wherein substantially all of the nucleotides of the sense strand and substantially all of the nucleotides of the antisense strand are modified nucleotides, wherein the sense strand is conjugated to a ligand attached at the 3′-terminus, and wherein the dsRNA agent is in a salt form.

In one embodiment, all of the nucleotides of the sense strand and all of the nucleotides of the antisense strand are modified nucleotides.

In one embodiment, the modified nucleotides are independently selected from the group consisting of a 2′-deoxy-2′-fluoro modified nucleotide, a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, and a non-natural base comprising nucleotide.

The region of complementarity may be at least 17 nucleotides in length or 19 nucleotides in length.

In one embodiment, the region of complementarity is between 19 and 21 nucleotides in length. In another embodiment, the region of complementarity is between 21 and 23 nucleotides in length.

In one embodiment, each strand is no more than 30 nucleotides in length.

At least one strand of the double stranded RNAi agent may comprise a 3′ overhang of at least 1 nucleotide or a 3′ overhang of at least 2 nucleotides, e.g., 2, 3, 4, 5, 6, 7, 9, 10, 11, 12, 13, 14, or 15 nucleotides. In other embodiments, at least one strand of the RNAi agent comprises a 5′ overhang of at least 1 nucleotide. In certain embodiments, at least one strand comprises a 5′ overhang of at least 2 nucleotides, e.g., 2, 3, 4, 5, 6, 7, 9, 10, 11, 12, 13, 14, or 15 nucleotides. In still other embodiments, both the 3′ and the 5′ end of one strand of the RNAi agent comprise an overhang of at least 1 nucleotide.

In certain embodiments, the ligand is an N-acetylgalactosamine (GalNAc). The ligand may be one or more GalNAc attached to the RNAi agent through a monovalent, a bivalent, or a trivalent branched linker. The ligand may be conjugated to the 3′ end of the sense strand of the double stranded RNAi agent, the 5′ end of the sense strand of the double stranded RNAi agent, the 3′ end of the antisense strand of the double stranded RNAi agent, or the 5′ end of the antisense strand of the double stranded RNAi agent.

In some embodiments, the double stranded RNAi agents comprise a plurality, e.g., 2, 3, 4, 5, or 6, of GalNAc, each independently attached to a plurality of nucleotides of the double stranded RNAi agent through a plurality of monovalent linkers.

In certain embodiments, the ligand is

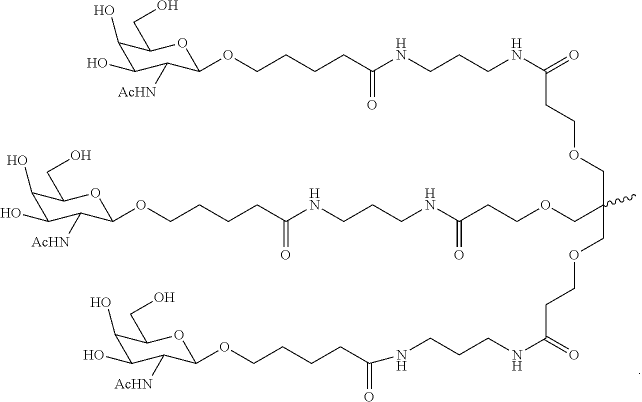 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In one embodiment, the RNAi agent is conjugated to the ligand via a linker and the ligand and linker are conjugated to the RNAi agent as shown in the following schematic

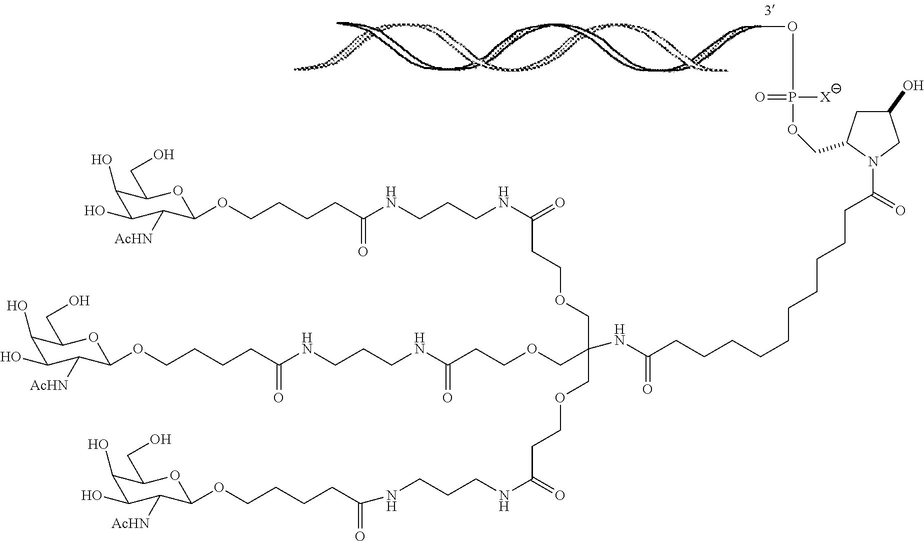

and, wherein X is O or S.

In one embodiment, the X is O.

In one embodiment, the region of complementarity consists of the nucleotide sequence of 5′-UUGAAGUAAAUGGUGUUAACCAG-3′(SEQ ID NO: 15).

In one embodiment, the double stranded RNAi agent comprises a sense strand comprising the nucleotide sequence of 5′-GGUUAACACCAUUUACUUCAA-3′(SEQ ID NO: 16), and an antisense strand comprising the nucleotide sequence of 5′-UUGAAGUAAAUGGUGUUAACCAG-3′(SEQ ID NO: 15).

In one embodiment, the sense strand comprises 5′-GfsgsUfuAfaCfaCfCfAfuUfuAfcUfuCfaAf-3′ (SEQ ID NO:13) and the antisense strand comprises 5′-usUfsgAfaGfuAfaAfuggUfgUfuAfaCfcsasg-3′ (SEQ ID NO:14), wherein a, c, g, and u are 2′-O-methyl (2′-OMe) A, C, G, or U; Af, Cf, Gf or Uf are 2′-fluoro A, C, G or U; and s is a phosphorothioate linkage.

In one embodiment, the sense strand comprises 5′-GfsgsUfuAfaCfaCfCfAfuUfuAfcUfuCfaAf-3′ (SEQ ID NO:13) and the antisense strand comprises 5′-usUfsgAfaGfuAfaAfuggUfgUfuAfaCfcsasg-3′ (SEQ ID NO:14), wherein a, c, g, and u are 2′-O-methyl (2′-OMe) A, C, G, or U; Af, Cf, Gf or Uf are 2′-fluoro A, C, G or U; and s is a phosphorothioate linkage; and wherein the sense strand is conjugated to the ligand via a linker and the ligand and linker are conjugated to the RNAi agent as shown in the following schematic

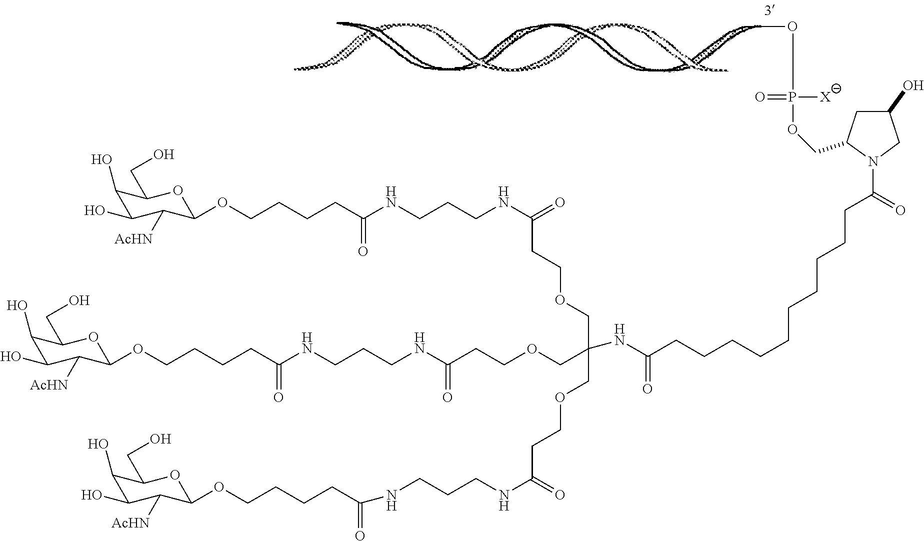

wherein X is O or S.

In one aspect, the present invention provides a pharmaceutical composition for inhibiting expression of a Serpinc1 gene. The pharmaceutical composition includes a double-stranded ribonucleic acid (dsRNA) agent at a concentration of about 50 mg/mL to about 200 mg/mL and phosphate buffered saline (PBS) at a concentration of about 1 mM to about 10 mM, wherein the pH and the osmolality of the pharmaceutical composition are suitable for subcutaneous administration to a subject, wherein the dsRNA agent has a sense strand consisting of the nucleotide sequence of 5′-GfsgsUfuAfaCfaCfCfAfuUfuAfcUfuCfaAf-3′ (SEQ ID NO:941) and an antisense strand consisting of the nucleotide sequence of 5′-usUfsgAfaGfuAfaAfuggUfgUfuAfaCfcsasg-3′ (SEQ ID NO:960), wherein a, g, c, and u are 2′-O-methyl (2′-OMe) A, G, C, and U; Af, Gf, Cf, and Uf are 2′-fluoro A, G, C, U; and s is a phosphorothioate linkage, and wherein a ligand is conjugated to the 3′ end of the sense strand via a linker, and wherein the ligand and the linker have the following structure:

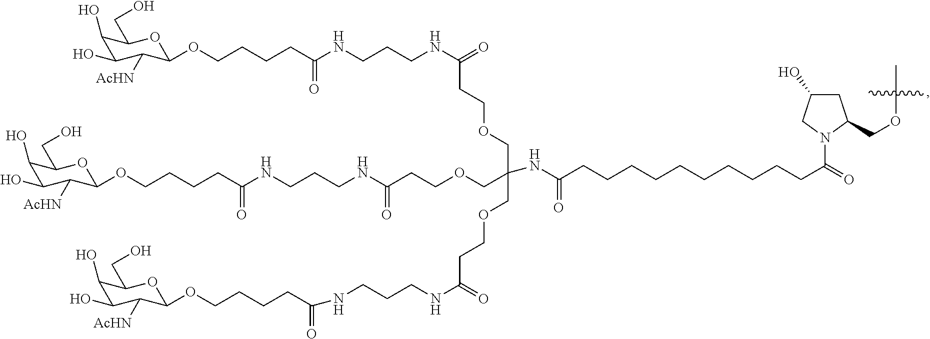 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

wherein the dsRNA agent is in a free acid form.

In another aspect, the present invention provides a pharmaceutical composition for inhibiting expression of a Serpinc1 gene. The pharmaceutical composition includes a double-stranded ribonucleic acid (dsRNA) agent at a concentration of about 50 mg/mL to about 200 mg/mL and phosphate buffered saline (PBS) at a concentration of about 1 mM to about 10 mM, wherein the pH and the osmolality of the pharmaceutical composition are suitable for subcutaneous administration to a subject, wherein the dsRNA agent has a sense strand consisting of the nucleotide sequence of 5′-GfsgsUfuAfaCfaCfCfAfuUfuAfcUfuCfaAf-3′ (SEQ ID NO:941) and an antisense strand consisting of the nucleotide sequence of 5′-usUfsgAfaGfuAfaAfuggUfgUfuAfaCfcsasg-3′ (SEQ ID NO:960), wherein a, g, c, and u are 2′-O-methyl (2′-OMe) A, G, C, and U; Af, Gf, Cf, and Uf are 2′-fluoro A, G, C, U; and s is a phosphorothioate linkage, and wherein a ligand is conjugated to the 3′ end of the sense strand via a linker, and wherein the ligand and the linker have the following structure:

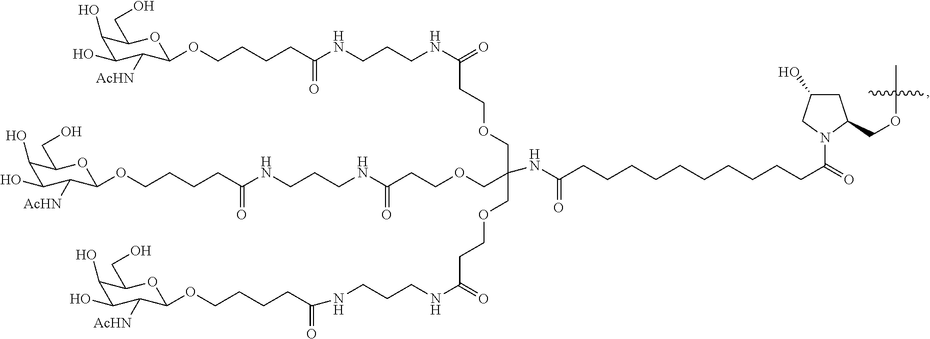 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

wherein the dsRNA agent is in a salt form.

In one aspect, the present invention provides a pharmaceutical composition for inhibiting expression of a Serpinc1 gene. The compositions include a double-stranded ribonucleic acid (dsRNA) agent at a concentration of about 100 mg/mL and phosphate buffered saline (PBS) at a concentration of about 5 mM, wherein the pH of the pharmaceutical composition is about 6.8 to about 7.2, wherein the osmolality of the pharmaceutical composition is about 300 mOsm/kg, wherein the dsRNA agent has a sense strand consisting of the nucleotide sequence of 5′-GfsgsUfuAfaCfaCfCfAfuUfuAfcUfuCfaAf-3′ (SEQ ID NO:941) and an antisense strand consisting of the nucleotide sequence of 5′-usUfsgAfaGfuAfaAfuggUfgUfuAfaCfcsasg-3′ (SEQ ID NO:960), wherein a, g, c, and u are 2′-O-methyl (2′-OMe) A, G, C, and U; Af, Gf, Cf, and Uf are 2′-fluoro A, G, C, U; and s is a phosphorothioate linkage, and wherein a ligand is conjugated to the 3′ end of the sense strand via a linker, and wherein the ligand and the linker have the following structure:

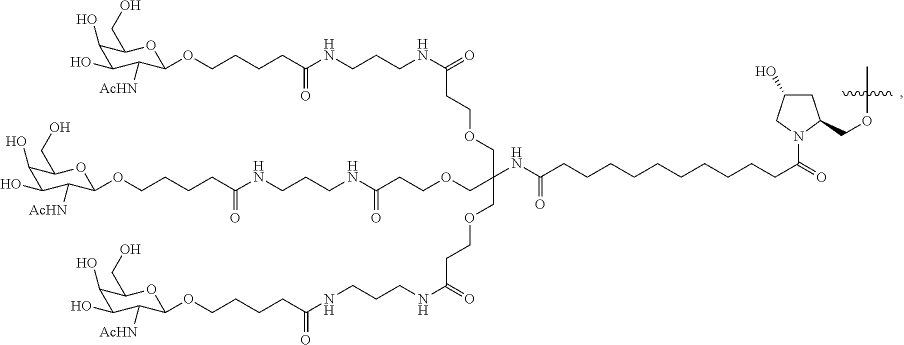 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

wherein the dsRNA agent is in a free acid form.

In another aspect, the present invention provides a pharmaceutical composition for inhibiting expression of a Serpinc1 gene. The pharmaceutical compositions include a double-stranded ribonucleic acid (dsRNA) agent at a concentration of about 106 mg/mL and phosphate buffered saline (PBS) at a concentration of about 5 mM, wherein the pH of the pharmaceutical composition is about 6.8 to about 7.2, wherein the osmolality of the pharmaceutical composition is about 300 mOsm/kg, wherein the dsRNA agent has a sense strand consisting of the nucleotide sequence of 5′-GfsgsUfuAfaCfaCfCfAfuUfuAfcUfuCfaAf-3′ (SEQ ID NO:941) and an antisense strand consisting of the nucleotide sequence of 5′-usUfsgAfaGfuAfaAfuggUfgUfuAfaCfcsasg-3′ (SEQ ID NO:960), wherein a, g, c, and u are 2′-O-methyl (2′-OMe) A, G, C, and U; Af, Gf, Cf, and Uf are 2′-fluoro A, G, C, U; and s is a phosphorothioate linkage, and wherein a ligand is conjugated to the 3′ end of the sense strand via a linker, and wherein the ligand and the linker have the following structure:

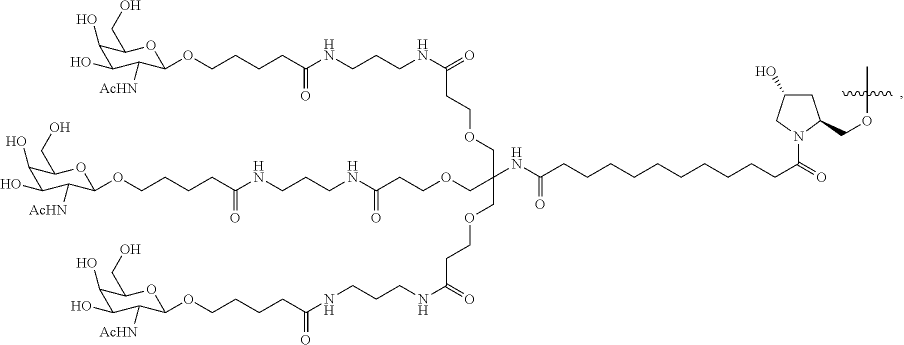 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

wherein the dsRNA agent is in a salt form.

The compositions of the present invention can additionally contain other adjunct components conventionally found in pharmaceutical compositions, at their art-established usage levels. Thus, for example, the compositions can contain additional, compatible, pharmaceutically-active materials such as, for example, antipruritics, astringents, local anesthetics or anti-inflammatory agents, or can contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

In some embodiments, pharmaceutical compositions featured in the invention include (a) one or more iRNA compounds and (b) one or more agents which function by a non-RNAi mechanism and which are useful in treating a hemolytic disorder. Examples of such agents include, but are not limited to an anti-inflammatory agent, anti-steatosis agent, anti-viral, and/or anti-fibrosis agent. In addition, other substances commonly used to protect the liver, such as silymarin, can also be used in conjunction with the iRNAs described herein. Other agents useful for treating liver diseases include telbivudine, entecavir, and protease inhibitors such as telaprevir and other disclosed, for example, in Tung et al., U.S. Application Publication Nos. 2005/0148548, 2004/0167116, and 2003/0144217; and in Hale et al., U.S. Application Publication No. 2004/0127488.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are preferred.

### III. iRNAs for Use in the Pharmaceutical Compositions of the Invention

The compositions of the invention include RNAi agents which target a Serpinc1 gene and inhibit the expression of the Serpinc1 gene in a cell, such as a cell within a subject, e.g., a mammal, such as a human having a Serpinc1-associated disorder, e.g., a bleeding disorder, e.g., hemophilia.

As used herein, “target sequence” refers to a contiguous portion of the nucleotide sequence of an mRNA molecule formed during the transcription of a Serpinc1 gene, including mRNA that is a product of RNA processing of a primary transcription product. In one embodiment, the target portion of the sequence will be at least long enough to serve as a substrate for iRNA-directed cleavage at or near that portion of the nucleotide sequence of an mRNA molecule formed during the transcription of a Serpinc1 gene.

The target sequence may be from about 9-36 nucleotides in length, e.g., about 15-30 nucleotides in length. For example, the target sequence can be from about 15-30 nucleotides, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides in length. Ranges and lengths intermediate to the above recited ranges and lengths are also contemplated to be part of the invention.

As used herein, the term “strand comprising a sequence” refers to an oligonucleotide comprising a chain of nucleotides that is described by the sequence referred to using the standard nucleotide nomenclature.

“G,” “C,” “A,” “T” and “U” each generally stand for a nucleotide that contains guanine, cytosine, adenine, thymidine and uracil as a base, respectively. However, it will be understood that the term “ribonucleotide” or “nucleotide” can also refer to a modified nucleotide, as further detailed below, or a surrogate replacement moiety (see, e.g., Table 1). The skilled person is well aware that guanine, cytosine, adenine, and uracil can be replaced by other moieties without substantially altering the base pairing properties of an oligonucleotide comprising a nucleotide bearing such replacement moiety. For example, without limitation, a nucleotide comprising inosine as its base can base pair with nucleotides containing adenine, cytosine, or uracil. Hence, nucleotides containing uracil, guanine, or adenine can be replaced in the nucleotide sequences of dsRNA featured in the invention by a nucleotide containing, for example, inosine. In another example, adenine and cytosine anywhere in the oligonucleotide can be replaced with guanine and uracil, respectively to form G-U Wobble base pairing with the target mRNA. Sequences containing such replacement moieties are suitable for the compositions and methods featured in the invention.

The terms “iRNA”, “RNAi agent,” “iRNA agent,”, “RNA interference agent” as used interchangeably herein, refer to an agent that contains RNA as that term is defined herein, and which mediates the targeted cleavage of an RNA transcript via an RNA-induced silencing complex (RISC) pathway. iRNA directs the sequence-specific degradation of mRNA through a process known as RNA interference (RNAi). The iRNA modulates, e.g., inhibits, the expression of Serpinc1 in a cell, e.g., a cell within a subject, such as a mammalian subject.

In one embodiment, an RNAi agent of the invention includes a single stranded RNA that interacts with a target RNA sequence, e.g., a Serpinc1 target mRNA sequence, to direct the cleavage of the target RNA. Without wishing to be bound by theory it is believed that long double stranded RNA introduced into cells is broken down into siRNA by a Type III endonuclease known as Dicer (Sharp et al. (2001) Genes Dev. 15:485). Dicer, a ribonuclease-III-like enzyme, processes the dsRNA into 19-23 base pair short interfering RNAs with characteristic two base 3′ overhangs (Bernstein, et al., (2001) Nature 409:363). The siRNAs are then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleave the target to induce silencing (Elbashir, et al., (2001) Genes Dev. 15:188). Thus, in one aspect the invention relates to a single stranded RNA (siRNA) generated within a cell and which promotes the formation of a RISC complex to effect silencing of the target gene, i.e., a Serpinc1 gene. Accordingly, the term “siRNA” is also used herein to refer to an RNAi as described above.

In another embodiment, the RNAi agent may be a single-stranded siRNA that is introduced into a cell or organism to inhibit a target mRNA. Single-stranded RNAi agents bind to the RISC endonuclease, Argonaute 2, which then cleaves the target mRNA. The single-stranded siRNAs are generally 15-30 nucleotides and are chemically modified. The design and testing of single-stranded siRNAs are described in U.S. Pat. No. 8,101,348 and in Lima et al., (2012) Cell 150: 883-894, the entire contents of each of which are hereby incorporated herein by reference. Any of the antisense nucleotide sequences described herein may be used as a single-stranded siRNA as described herein or as chemically modified by the methods described in Lima et al., (2012) Cell 150; 883-894.

In another embodiment, an “iRNA” for use in the compositions, uses, and methods of the invention is a double stranded RNA and is referred to herein as a “double stranded RNAi agent,” “double stranded RNA (dsRNA) molecule,” “dsRNA agent,” or “dsRNA”. The term “dsRNA”, refers to a complex of ribonucleic acid molecules, having a duplex structure comprising two anti-parallel and substantially complementary nucleic acid strands, referred to as having “sense” and “antisense” orientations with respect to a target RNA, i.e., a Serpinc1 gene. In some embodiments of the invention, a double stranded RNA (dsRNA) triggers the degradation of a target RNA, e.g., an mRNA, through a post-transcriptional gene-silencing mechanism referred to herein as RNA interference or RNAi.

In general, the majority of nucleotides of each strand of a dsRNA molecule are ribonucleotides, but as described in detail herein, each or both strands can also include one or more non-ribonucleotides, e.g., a deoxyribonucleotide and/or a modified nucleotide. In addition, as used in this specification, an “RNAi agent” may include ribonucleotides with chemical modifications; an RNAi agent may include substantial modifications at multiple nucleotides.

As used herein, the term “modified nucleotide” refers to a nucleotide having, independently, a modified sugar moiety, a modified internucleotide linkage, and/or a modified nucleobase. Thus, the term modified nucleotide encompasses substitutions, additions or removal of, e.g., a functional group or atom, to internucleoside linkages, sugar moieties, or nucleobases.

The modifications suitable for use in the agents of the invention include all types of modifications disclosed herein or known in the art. Any such modifications, as used in a siRNA type molecule, are encompassed by “RNAi agent” for the purposes of this specification and claims.

The duplex region may be of any length that permits specific degradation of a desired target RNA through a RISC pathway, and may range from about 9 to 36 base pairs in length, e.g., about 15-30 base pairs in length, for example, about 9, 10, 11, 12, 13, 14, 15, 16, 17, 18, 19, 20, 21, 22, 23, 24, 25, 26, 27, 28, 29, 30, 31, 32, 33, 34, 35, or 36 base pairs in length, such as about 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 base pairs in length. Ranges and lengths intermediate to the above recited ranges and lengths are also contemplated to be part of the invention.

The two strands forming the duplex structure may be different portions of one larger RNA molecule, or they may be separate RNA molecules. Where the two strands are part of one larger molecule, and therefore are connected by an uninterrupted chain of nucleotides between the 3′-end of one strand and the 5′-end of the respective other strand forming the duplex structure, the connecting RNA chain is referred to as a “hairpin loop.” A hairpin loop can comprise at least one unpaired nucleotide. In some embodiments, the hairpin loop can comprise at least 2, at least 3, at least 4, at least 5, at least 6, at least 7, at least 8, at least 9, at least 10, at least 20, at least 23 or more unpaired nucleotides.

Where the two substantially complementary strands of a dsRNA are comprised by separate RNA molecules, those molecules need not, but can be covalently connected. Where the two strands are connected covalently by means other than an uninterrupted chain of nucleotides between the 3′-end of one strand and the 5′-end of the respective other strand forming the duplex structure, the connecting structure is referred to as a “linker.” The RNA strands may have the same or a different number of nucleotides. The maximum number of base pairs is the number of nucleotides in the shortest strand of the dsRNA minus any overhangs that are present in the duplex. In addition to the duplex structure, an RNAi may comprise one or more nucleotide overhangs.

In one embodiment, an RNAi agent of the invention is a dsRNA of 24-30 nucleotides that interacts with a target RNA sequence, e.g., a Serpinc1 target mRNA sequence, to direct the cleavage of the target RNA. Without wishing to be bound by theory, long double stranded RNA introduced into cells is broken down into siRNA by a Type III endonuclease known as Dicer (Sharp et al. (2001) Genes Dev. 15:485). Dicer, a ribonuclease-III-like enzyme, processes the dsRNA into 19-23 base pair short interfering RNAs with characteristic two base 3′ overhangs (Bernstein, et al., (2001) Nature 409:363). The siRNAs are then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleave the target to induce silencing (Elbashir, et al., (2001) Genes Dev. 15:188).

As used herein, the term “nucleotide overhang” refers to at least one unpaired nucleotide that protrudes from the duplex structure of an iRNA, e.g., a dsRNA. For example, when a 3′-end of one strand of a dsRNA extends beyond the 5′-end of the other strand, or vice versa, there is a nucleotide overhang. A dsRNA can comprise an overhang of at least one nucleotide; alternatively the overhang can comprise at least two nucleotides, at least three nucleotides, at least four nucleotides, at least five nucleotides or more. A nucleotide overhang can comprise or consist of a nucleotide/nucleoside analog, including a deoxynucleotide/nucleoside. The overhang(s) can be on the sense strand, the antisense strand or any combination thereof.

Furthermore, the nucleotide(s) of an overhang can be present on the 5′-end, 3′-end or both ends of either an antisense or sense strand of a dsRNA.

In one embodiment, the antisense strand of a dsRNA has a 1-10 nucleotide, e.g., a 1, 2, 3, 4, 5, 6, 7, 8, 9, or 10 nucleotide, overhang at the 3′-end and/or the 5′-end. In one embodiment, the sense strand of a dsRNA has a 1-10 nucleotide, e.g., a 1, 2, 3, 4, 5, 6, 7, 8, 9, or 10 nucleotide, overhang at the 3′-end and/or the 5′-end. In another embodiment, one or more of the nucleotides in the overhang is replaced with a nucleoside thiophosphate.

In certain embodiments, the overhang on the sense strand or the antisense strand, or both, can include extended lengths longer than 10 nucleotides, e.g., 10-30 nucleotides, 10-25 nucleotides, 10-20 nucleotides or 10-15 nucleotides in length. In certain embodiments, an extended overhang is on the sense strand of the duplex. In certain embodiments, an extended overhang is present on the 3′end of the sense strand of the duplex. In certain embodiments, an extended overhang is present on the 5′end of the sense strand of the duplex. In certain embodiments, an extended overhang is on the antisense strand of the duplex. In certain embodiments, an extended overhang is present on the 3′end of the antisense strand of the duplex. In certain embodiments, an extended overhang is present on the 5′end of the antisense strand of the duplex. In certain embodiments, one or more of the nucleotides in the extended overhang is replaced with a nucleoside thiophosphate.

“Blunt” or “blunt end” means that there are no unpaired nucleotides at that end of the double stranded RNAi agent, i.e., no nucleotide overhang. A “blunt ended” RNAi agent is a dsRNA that is double stranded over its entire length, i.e., no nucleotide overhang at either end of the molecule. The RNAi agents of the invention include RNAi agents with nucleotide overhangs at one end (i.e., agents with one overhang and one blunt end) or with nucleotide overhangs at both ends.

The term “antisense strand” or “guide strand” refers to the strand of an iRNA, e.g., a dsRNA, which includes a region that is substantially complementary to a target sequence, e.g., a Serpinc1 mRNA. As used herein, the term “region of complementarity” refers to the region on the antisense strand that is substantially complementary to a sequence, for example a target sequence, e.g., a Serpinc1 nucleotide sequence, as defined herein. Where the region of complementarity is not fully complementary to the target sequence, the mismatches can be in the internal or terminal regions of the molecule. Generally, the most tolerated mismatches are in the terminal regions, e.g., within 5, 4, 3, or 2 nucleotides of the 5′- and/or 3′-terminus of the iRNA.

The term “sense strand,” or “passenger strand” as used herein, refers to the strand of an iRNA that includes a region that is substantially complementary to a region of the antisense strand as that term is defined herein.

As used herein, the term “cleavage region” refers to a region that is located immediately adjacent to the cleavage site. The cleavage site is the site on the target at which cleavage occurs. In some embodiments, the cleavage region comprises three bases on either end of, and immediately adjacent to, the cleavage site. In some embodiments, the cleavage region comprises two bases on either end of, and immediately adjacent to, the cleavage site. In some embodiments, the cleavage site specifically occurs at the site bound by nucleotides 10 and 11 of the antisense strand, and the cleavage region comprises nucleotides 11, 12 and 13.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions can include: 400 mM NaCl, 40 mM PIPES pH 6.4, 1 mM EDTA, 50° C. or 70° C. for 12-16 hours followed by washing (see, e.g., “Molecular Cloning: A Laboratory Manual, Sambrook, et al. (1989) Cold Spring Harbor Laboratory Press). Other conditions, such as physiologically relevant conditions as can be encountered inside an organism, can apply. The skilled person will be able to determine the set of conditions most appropriate for a test of complementarity of two sequences in accordance with the ultimate application of the hybridized nucleotides.

Complementary sequences within an iRNA, e.g., within a dsRNA as described herein, include base-pairing of the oligonucleotide or polynucleotide comprising a first nucleotide sequence to an oligonucleotide or polynucleotide comprising a second nucleotide sequence over the entire length of one or both nucleotide sequences. Such sequences can be referred to as “fully complementary” with respect to each other herein. However, where a first sequence is referred to as “substantially complementary” with respect to a second sequence herein, the two sequences can be fully complementary, or they can form one or more, but generally not more than 5, 4, 3 or 2 mismatched base pairs upon hybridization for a duplex up to 30 base pairs, while retaining the ability to hybridize under the conditions most relevant to their ultimate application, e.g., inhibition of gene expression via a RISC pathway. However, where two oligonucleotides are designed to form, upon hybridization, one or more single stranded overhangs, such overhangs shall not be regarded as mismatches with regard to the determination of complementarity. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, can yet be referred to as “fully complementary” for the purposes described herein.

“Complementary” sequences, as used herein, can also include, or be formed entirely from, non-Watson-Crick base pairs and/or base pairs formed from non-natural and modified nucleotides, in so far as the above requirements with respect to their ability to hybridize are fulfilled. Such non-Watson-Crick base pairs include, but are not limited to, G:U Wobble or Hoogstein base pairing.

The terms “complementary,” “fully complementary” and “substantially complementary” herein can be used with respect to the base matching between the sense strand and the antisense strand of a dsRNA, or between the antisense strand of an iRNA agent and a target sequence, as will be understood from the context of their use.

As used herein, a polynucleotide that is “substantially complementary to at least part of” a messenger RNA (mRNA) refers to a polynucleotide that is substantially complementary to a contiguous portion of the mRNA of interest (e.g., an mRNA encoding Serpinc1). For example, a polynucleotide is complementary to at least a part of a Serpinc1 mRNA if the sequence is substantially complementary to a non-interrupted portion of an mRNA encoding Serpinc1.

Accordingly, in some embodiments, the antisense strand polynucleotides disclosed herein are fully complementary to the target Serpinc1 sequence. In other embodiments, the antisense strand polynucleotides disclosed herein are substantially complementary to the target Serpinc1 sequence and comprise a contiguous nucleotide sequence which is at least about 80% complementary over its entire length to the equivalent region of the nucleotide sequence of SEQ ID NO:1, or a fragment of SEQ ID NO:1, such as about 85%, about 86%, about 87%, about 88%, about 89%, about 90%, about % 91%, about 92%, about 93%, about 94%, about 95%, about 96%, about 97%, about 98%, or about 99% complementary.

In one embodiment, an RNAi agent of the invention includes a sense strand that is substantially complementary to an antisense polynucleotide which, in turn, is complementary to a target Serpinc1 sequence, and wherein the sense strand polynucleotide comprises a contiguous nucleotide sequence which is at least about 80% complementary over its entire length to the equivalent region of the nucleotide sequence of SEQ ID NO:5, or a fragment of any one of SEQ ID NO:5, such as about 85%, about 86%, about 87%, about 88%, about 89%, about 90%, about % 91%, about 92%, about 93%, about 94%, about 95%, about 96%, about 97%, about 98%, or about 99% complementary.

Suitable dsRNA agents capable of inhibiting the expression of a target gene (i.e., a Serpinc1 gene) in vivo include chemical modifications. In certain aspects of the invention, substantially all of the nucleotides of an iRNA of the invention are modified. In other embodiments of the invention, all of the nucleotides of an iRNA of the invention are modified. iRNAs of the invention in which “substantially all of the nucleotides are modified” are largely but not wholly modified and can include not more than 5, 4, 3, 2, or 1 unmodified nucleotides.

The iRNA agents for use in the methods of the invention generally include an RNA strand (the antisense strand) having a region which is about 30 nucleotides or less in length, e.g., 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 nucleotides in length, which region is substantially complementary to at least part of an mRNA transcript of an Serpinc1 gene.

In other embodiments, one or both of the strands of the double stranded RNAi agents of the invention is up to 66 nucleotides in length, e.g., 36-66, 26-36, 25-36, 31-60, 22-43, 27-53 nucleotides in length, with a region of at least 19 contiguous nucleotides that is substantially complementary to at least a part of an mRNA transcript of a Serpinc1 gene. In some embodiments, the sense and antisense strands form a duplex of 18-30 contiguous nucleotides.

In some embodiments, the iRNA agents for use in the methods of the invention include an RNA strand (the antisense strand) which can be up to 66 nucleotides in length, e.g., 36-66, 26-36, 25-36, 31-60, 22-43, 27-53 nucleotides in length, with a region of at least 19 contiguous nucleotides that is substantially complementary to at least a part of an mRNA transcript of a Serpinc1 gene. In some embodiments, such iRNA agents having longer length antisense strands may include a second RNA strand (the sense strand) of 20-60 nucleotides in length wherein the sense and antisense strands form a duplex of 18-30 contiguous nucleotides.

The RNAi agent comprises a sense strand and an antisense strand. Each strand of the RNAi agent may range from 12-30 nucleotides in length. For example, each strand may be between 14-30 nucleotides in length, 17-30 nucleotides in length, 19-30 nucleotides in length, 25-30 nucleotides in length, 27-30 nucleotides in length, 17-23 nucleotides in length, 17-21 nucleotides in length, 17-19 nucleotides in length, 19-25 nucleotides in length, 19-23 nucleotides in length, 19-21 nucleotides in length, 21-25 nucleotides in length, or 21-23 nucleotides in length.

The sense strand and antisense strand typically form a duplex double stranded RNA (“dsRNA”), also referred to herein as an “RNAi agent.” The duplex region of an RNAi agent may be 12-30 nucleotide pairs in length. For example, the duplex region can be between 14-30 nucleotide pairs in length, 17-30 nucleotide pairs in length, 27-30 nucleotide pairs in length, 17-23 nucleotide pairs in length, 17-21 nucleotide pairs in length, 17-19 nucleotide pairs in length, 19-25 nucleotide pairs in length, 19-23 nucleotide pairs in length, 19-21 nucleotide pairs in length, 21-25 nucleotide pairs in length, or 21-23 nucleotide pairs in length. In another example, the duplex region is selected from 15, 16, 17, 18, 19, 20, 21, 22, 23, 24, 25, 26, and 27 nucleotides in length.

In one embodiment, the RNAi agent may contain one or more overhang regions and/or capping groups at the 3′-end, 5′-end, or both ends of one or both strands. The overhang can be 1-6 nucleotides in length, for instance 2-6 nucleotides in length, 1-5 nucleotides in length, 2-5 nucleotides in length, 1-4 nucleotides in length, 2-4 nucleotides in length, 1-3 nucleotides in length, 2-3 nucleotides in length, or 1-2 nucleotides in length. The overhangs can be the result of one strand being longer than the other, or the result of two strands of the same length being staggered. The overhang can form a mismatch with the target mRNA or it can be complementary to the gene sequences being targeted or can be another sequence. The first and second strands can also be joined, e.g., by additional bases to form a hairpin, or by other non-base linkers.

In one embodiment, the nucleotides in the overhang region of the RNAi agent can each independently be a modified or unmodified nucleotide including, but no limited to 2′-sugar modified, such as, 2-F, 2′-O-methyl, thymidine (T), 2′-O-methoxyethyl-5-methyluridine (Teo), 2′-O-methoxyethyladenosine (Aeo), 2′-O-methoxyethyl-5-methylcytidine (m5Ceo), and any combinations thereof. For example, TT can be an overhang sequence for either end on either strand. The overhang can form a mismatch with the target mRNA or it can be complementary to the gene sequences being targeted or can be another sequence.

The 5′- or 3′-overhangs at the sense strand, antisense strand or both strands of the RNAi agent may be phosphorylated. In some embodiments, the overhang region(s) contains two nucleotides having a phosphorothioate between the two nucleotides, where the two nucleotides can be the same or different. In one embodiment, the overhang is present at the 3′-end of the sense strand, antisense strand, or both strands. In one embodiment, this 3′-overhang is present in the antisense strand. In one embodiment, this 3′-overhang is present in the sense strand.

The RNAi agent may contain only a single overhang, which can strengthen the interference activity of the RNAi, without affecting its overall stability. For example, the single-stranded overhang may be located at the 3′-terminal end of the sense strand or, alternatively, at the 3′-terminal end of the antisense strand. The RNAi may also have a blunt end, located at the 5′-end of the antisense strand (or the 3′-end of the sense strand) or vice versa. Generally, the antisense strand of the RNAi has a nucleotide overhang at the 3′-end, and the 5′-end is blunt. While not wishing to be bound by theory, the asymmetric blunt end at the 5′-end of the antisense strand and 3′-end overhang of the antisense strand favor the guide strand loading into RISC process.

Any of the nucleic acids featured in the invention can be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference. Modifications include, for example, end modifications, e.g., 5′-end modifications (phosphorylation, conjugation, inverted linkages) or 3′-end modifications (conjugation, DNA nucleotides, inverted linkages, etc.); base modifications, e.g., replacement with stabilizing bases, destabilizing bases, or bases that base pair with an expanded repertoire of partners, removal of bases (abasic nucleotides), or conjugated bases; sugar modifications (e.g., at the 2′-position or 4′-position) or replacement of the sugar; and/or backbone modifications, including modification or replacement of the phosphodiester linkages. Specific examples of iRNA compounds useful in the embodiments described herein include, but are not limited to RNAs containing modified backbones or no natural internucleoside linkages. RNAs having modified backbones include, among others, those that do not have a phosphorus atom in the backbone. For the purposes of this specification, and as sometimes referenced in the art, modified RNAs that do not have a phosphorus atom in their internucleoside backbone can also be considered to be oligonucleosides. In some embodiments, a modified iRNA will have a phosphorus atom in its internucleoside backbone.

Modified RNA backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′-linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464, the entire contents of each of which are hereby incorporated herein by reference.

Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and, 5,677,439, the entire contents of each of which are hereby incorporated herein by reference.

In other embodiments, suitable RNA mimetics are contemplated for use in iRNAs, in which both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262, the entire contents of each of which are hereby incorporated herein by reference. Additional PNA compounds suitable for use in the iRNAs of the invention are described in, for example, in Nielsen et al., Science, 1991, 254, 1497-1500.

Some embodiments featured in the invention include RNAs with phosphorothioate backbones and oligonucleosides with heteroatom backbones, and in particular —CH₂—NH—CH₂—, —CH₂—N(CH₃)—O—CH₂-[known as a methylene (methylimino) or MMI backbone], —CH₂—O—N(CH₃)—CH₂-, —CH₂—N(CH₃)—N(CH₃)—CH₂- and —N(CH₃)—CH₂—CH₂-[wherein the native phosphodiester backbone is represented as —O—P—O—CH₂—] of the above-referenced U.S. Pat. No. 5,489,677, and the amide backbones of the above-referenced U.S. Pat. No. 5,602,240. In some embodiments, the RNAs featured herein have morpholino backbone structures of the above-referenced U.S. Pat. No. 5,034,506.

Modified RNAs can also contain one or more substituted sugar moieties. The iRNAs, e.g., dsRNAs, featured herein can include one of the following at the 2′-position: OH; F; O-, S-, or N-alkyl; O-, S-, or N-alkenyl; O-, S- or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl. Exemplary suitable modifications include O[(CH₂)ₙO]ₘCH₃, O(CH₂).ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. In other embodiments, dsRNAs include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an iRNA, or a group for improving the pharmacodynamic properties of an iRNA, and other substituents having similar properties. In some embodiments, the modification includes a 2′-methoxyethoxy (2′-O—CH₂CH₂OCH₃, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂.

Other modifications include 2′-methoxy (2′-OCH₃), 2′-aminopropoxy (2′-OCH₂CH₂CH₂NH₂) and 2′-fluoro (2′-F). Similar modifications can also be made at other positions on the RNA of an iRNA, particularly the 3′ position of the sugar on the 3′ terminal nucleotide or in 2′-5′ linked dsRNAs and the 5′ position of 5′ terminal nucleotide. iRNAs can also have sugar mimetics such as cyclobutyl moieties in place of the pentofuranosyl sugar.

Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, certain of which are commonly owned with the instant application. The entire contents of each of the foregoing are hereby incorporated herein by reference.

An iRNA can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as deoxy-thymine (dT), 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Further nucleobases include those disclosed in U.S. Pat. No. 3,687,808, those disclosed in Modified Nucleosides in Biochemistry, Biotechnology and Medicine, Herdewijn, P. ed. Wiley-VCH, 2008; those disclosed in The Concise Encyclopedia Of Polymer Science And Engineering, pages 858-859, Kroschwitz, J. L, ed. John Wiley & Sons, 1990, these disclosed by Englisch et al., Angewandte Chemie, International Edition, 1991, 30, 613, and those disclosed by Sanghvi, Y S., Chapter 15, dsRNA Research and Applications, pages 289-302, Crooke, S. T. and Lebleu, B., Ed., CRC Press, 1993. Certain of these nucleobases are particularly useful for increasing the binding affinity of the oligomeric compounds featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications.

Representative U.S. patents that teach the preparation of certain of the above noted modified nucleobases as well as other modified nucleobases include, but are not limited to, the above noted U.S. Pat. Nos. 3,687,808, 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; 5,750,692; 6,015,886; 6,147,200; 6,166,197; 6,222,025; 6,235,887; 6,380,368; 6,528,640; 6,639,062; 6,617,438; 7,045,610; 7,427,672; and 7,495,088, the entire contents of each of which are hereby incorporated herein by reference.

The RNA of an iRNA can also be modified to include one or more bicyclic sugar moities. A “bicyclic sugar” is a furanosyl ring modified by the bridging of two atoms. A “bicyclic nucleoside” (“BNA”) is a nucleoside having a sugar moiety comprising a bridge connecting two carbon atoms of the sugar ring, thereby forming a bicyclic ring system. In certain embodiments, the bridge connects the 4′-carbon and the 2′-carbon of the sugar ring. Thus, in some embodiments an agent of the invention may include the RNA of an iRNA can also be modified to include one or more locked nucleic acids (LNA). A locked nucleic acid is a nucleotide having a modified ribose moiety in which the ribose moiety comprises an extra bridge connecting the 2′ and 4′ carbons. In other words, an LNA is a nucleotide comprising a bicyclic sugar moiety comprising a 4′-CH2-O-2′ bridge. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mol Canc Ther 6(3):833-843; Grunweller, A. et al., (2003) Nucleic Acids Research 31(12):3185-3193).

Examples of bicyclic nucleosides for use in the polynucleotides of the invention include without limitation nucleosides comprising a bridge between the 4′ and the 2′ ribosyl ring atoms. In certain embodiments, the antisense polynucleotide agents of the invention include one or more bicyclic nucleosides comprising a 4′ to 2′ bridge. Examples of such 4′ to 2′ bridged bicyclic nucleosides, include but are not limited to 4′-(CH2)-O-2′ (LNA); 4′-(CH2)-S-2′; 4′-(CH2)2-O-2′ (ENA); 4′-CH(CH3)-O-2′ (also referred to as “constrained ethyl” or “cEt”) and 4′-CH(CH2OCH3)-O-2′ (and analogs thereof; see, e.g., U.S. Pat. No. 7,399,845); 4′-C(CH3)(CH3)O-2′ (and analogs thereof; see e.g., U.S. Pat. No. 8,278,283); 4′-CH2-N(OCH3)-2′ (and analogs thereof; see e.g., U.S. Pat. No. 8,278,425); 4′-CH2-O—N(CH3)-2′ (see, e.g., U.S. Patent Publication No. 2004/0171570); 4′-CH2-N(R)—O-2′, wherein R is H, C1-C12 alkyl, or a protecting group (see, e.g., U.S. Pat. No. 7,427,672); 4′-CH2-C(H)(CH3)-2′ (see, e.g., Chattopadhyaya et al., J. Org. Chem., 2009, 74, 118-134); and 4′-CH2-C(═CH2)-2′ (and analogs thereof; see, e.g., U.S. Pat. No. 8,278,426). The entire contents of each of the foregoing are hereby incorporated herein by reference.

Additional representative U.S. patents and US patent Publications that teach the preparation of locked nucleic acid nucleotides include, but are not limited to, the following: U.S. Pat. Nos. 6,268,490; 6,525,191; 6,670,461; 6,770,748; 6,794,499; 6,998,484; 7,053,207; 7,034,133; 7,084,125; 7,399,845; 7,427,672; 7,569,686; 7,741,457; 8,022,193; 8,030,467; 8,278,425; 8,278,426; 8,278,283; US 2008/0039618; and US 2009/0012281, the entire contents of each of which are hereby incorporated herein by reference.

Any of the foregoing bicyclic nucleosides can be prepared having one or more stereochemical sugar configurations including for example α-L-ribofuranose and 3-D-ribofuranose (see WO 99/14226).

The RNA of an iRNA can also be modified to include one or more constrained ethyl nucleotides. As used herein, a “constrained ethyl nucleotide” or “cEt” is a locked nucleic acid comprising a bicyclic sugar moiety comprising a 4′-CH(CH3)-O-2′ bridge. In one embodiment, a constrained ethyl nucleotide is in the S conformation referred to herein as “S-cEt.”

An iRNA of the invention may also include one or more “conformationally restricted nucleotides” (“CRN”). CRN are nucleotide analogs with a linker connecting the C2′ and C4′ carbons of ribose or the C3 and —C5′ carbons of ribose. CRN lock the ribose ring into a stable conformation and increase the hybridization affinity to mRNA. The linker is of sufficient length to place the oxygen in an optimal position for stability and affinity resulting in less ribose ring puckering.

Representative publications that teach the preparation of certain of the above noted CRN include, but are not limited to, US Patent Publication No. 2013/0190383; and PCT publication WO 2013/036868, the entire contents of each of which are hereby incorporated herein by reference.

One or more of the nucleotides of an iRNA of the invention may also include a hydroxymethyl substituted nucleotide. A “hydroxymethyl substituted nucleotide” is an acyclic 2′-3′-seco-nucleotide, also referred to as an “unlocked nucleic acid” (“UNA”) modification Representative U.S. publications that teach the preparation of UNA include, but are not limited to, U.S. Pat. No. 8,314,227; and US Patent Publication Nos. 2013/0096289; 2013/0011922; and 2011/0313020, the entire contents of each of which are hereby incorporated herein by reference.

Potentially stabilizing modifications to the ends of RNA molecules can include N-(acetylaminocaproyl)-4-hydroxyprolinol (Hyp-C6-NHAc), N-(caproyl-4-hydroxyprolinol (Hyp-C6), N-(acetyl-4-hydroxyprolinol (Hyp-NHAc), thymidine-2′-O-deoxythymidine (ether), N-(aminocaproyl)-4-hydroxyprolinol (Hyp-C6-amino), 2-docosanoyl-uridine-3″-phosphate, inverted base dT(idT) and others. Disclosure of this modification can be found in PCT Publication No. WO 2011/005861.

In certain aspects of the invention, the double stranded RNAi agents of the invention include agents with chemical modifications as disclosed, for example, in U.S. Provisional Application No. 61/561,710, filed on Nov. 18, 2011, or in PCT/US2012/065691, filed on Nov. 16, 2012, the entire contents of each of which are incorporated herein by reference.

The double stranded RNA (dsRNA) agents of the invention may optionally be conjugated to one or more ligands. The ligand can be attached to the sense strand, antisense strand or both strands, at the 3′-end, 5′-end or both ends. For instance, the ligand may be conjugated to the sense strand. In preferred embodiments, the ligand is conjugated to the 3′-end of the sense strand.

In one embodiment, the ligand is a carbohydrate conjugate, such as a monosaccharide. In one embodiment, the ligand is an N-acetylgalactosamine (GalNAc) GalNAc or GalNAc derivative. In certain embodiments of the invention, the GalNAc or GalNAc derivative is attached to an iRNA agent of the invention via a monovalent linker. In some embodiments, the GalNAc or GalNAc derivative is attached to an iRNA agent of the invention via a bivalent linker. In yet other embodiments of the invention, the GalNAc or GalNAc derivative is attached to an iRNA agent of the invention via a trivalent linker. Suitable ligands are disclosed in, for example, U.S. patent application Ser. No. 15/371,300 and U.S. Patent Publication No. 2009/0239814, the entire contents of each of which as they relate to suitable ligands are incorporated herein by reference.

In some embodiments, the ligand, e.g., GalNAc ligand, is attached to the 3′ end of the RNAi agent. In one embodiment, the RNAi agent is conjugated to the ligand via a linker, e.g., GalNAc ligand, as shown in the following schematic

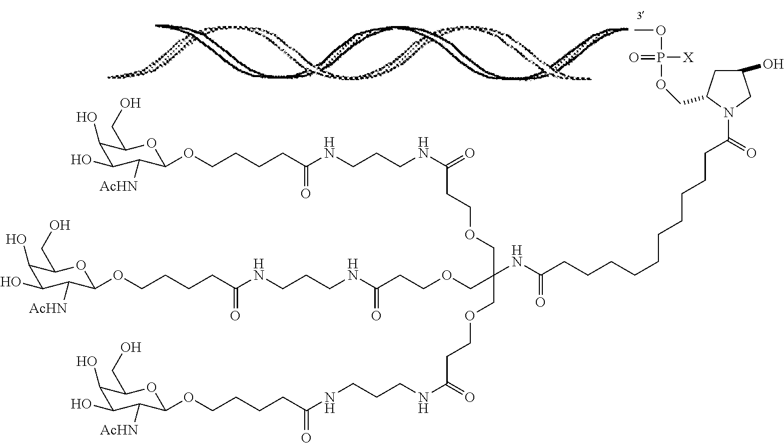

wherein X is O or S. In one embodiment, X is O.

Representative U.S. patents that teach the preparation of RNA conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941; 6,294,664; 6,320,017; 6,576,752; 6,783,931; 6,900,297; 7,037,646; 8,106,022, the entire contents of each of which are hereby incorporated herein by reference.

It is not necessary for all positions in a given compound to be uniformly modified, and in fact more than one of the aforementioned modifications can be incorporated in a single compound or even at a single nucleoside within an iRNA. The present invention also includes iRNA compounds that are chimeric compounds.

“Chimeric” iRNA compounds or “chimeras,” in the context of this invention, are iRNA compounds, preferably dsRNAs, which contain two or more chemically distinct regions, each made up of at least one monomer unit, i.e., a nucleotide in the case of a dsRNA compound.

These iRNAs typically contain at least one region wherein the RNA is modified so as to confer upon the iRNA increased resistance to nuclease degradation, increased cellular uptake, and/or increased binding affinity for the target nucleic acid. An additional region of the iRNA can serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of iRNA inhibition of gene expression. Consequently, comparable results can often be obtained with shorter iRNAs when chimeric dsRNAs are used, compared to phosphorothioate deoxy dsRNAs hybridizing to the same target region. Cleavage of the RNA target can be routinely detected by gel electrophoresis and, if necessary, associated nucleic acid hybridization techniques known in the art.

In certain instances, the RNA of an iRNA can be modified by a non-ligand group. A number of non-ligand molecules have been conjugated to iRNAs in order to enhance the activity, cellular distribution or cellular uptake of the iRNA, and procedures for performing such conjugations are available in the scientific literature. Such non-ligand moieties have included lipid moieties, such as cholesterol (Kubo, T. et al., Biochem. Biophys. Res. Comm., 2007, 365(1):54-61; Letsinger et al., Proc. Natl. Acad. Sci. USA, 1989, 86:6553), cholic acid (Manoharan et al., Bioorg. Med. Chem. Lett., 1994, 4:1053), a thioether, e.g., hexyl-S-tritylthiol (Manoharan et al., Ann. N.Y. Acad. Sci., 1992, 660:306; Manoharan et al., Bioorg. Med. Chem. Let., 1993, 3:2765), a thiocholesterol (Oberhauser et al., Nucl. Acids Res., 1992, 20:533), an aliphatic chain, e.g., dodecandiol or undecyl residues (Saison-Behmoaras et al., EMBO J., 1991, 10:111; Kabanov et al., FEBS Lett., 1990, 259:327; Svinarchuk et al., Biochimie, 1993, 75:49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36:3651; Shea et al., Nucl. Acids Res., 1990, 18:3777), a polyamine or a polyethylene glycol chain (Manoharan et al., Nucleosides & Nucleotides, 1995, 14:969), or adamantane acetic acid (Manoharan et al., Tetrahedron Lett., 1995, 36:3651), a palmityl moiety (Mishra et al., Biochim. Biophys. Acta, 1995, 1264:229), or an octadecylamine or hexylamino-carbonyl-oxycholesterol moiety (Crooke et al., J. Pharmacol. Exp. Ther., 1996, 277:923). Representative United States patents that teach the preparation of such RNA conjugates have been listed above. Typical conjugation protocols involve the synthesis of an RNAs bearing an amino linker at one or more positions of the sequence. The amino group is then reacted with the molecule being conjugated using appropriate coupling or activating reagents. The conjugation reaction can be performed either with the RNA still bound to the solid support or following cleavage of the RNA, in solution phase. Purification of the RNA conjugate by HPLC typically affords the pure conjugate.

### V. Uses of the Pharmaceutical Compositions of the Invention

The pharmaceutical compositions of the invention are useful for therapeutic and prophylactic treatment of subjects having a disorder that would benefit from reduction in Serpinc1 expression, such as a bleeding disorder, e.g., a hemophilia (e.g., hemophilia A, hemophilia B, or hemophilia C).

As used herein, the terms “treating” or “treatment” refer to a beneficial or desired result including, but not limited to, alleviation or amelioration of one or more symptoms, diminishing the extent of bleeding, stabilized (i.e., not worsening) state of bleeding, amelioration or palliation of the bleeding, whether detectable or undetectable, or resolving the bleeding. “Treatment” can also mean prolonging survival as compared to expected survival in the absence of treatment. In the methods of the invention, treatment includes on demand treatment and control of bleeding episodes, perioperative management of bleeding and routine prophylaxis to reduce the frequency of bleeding episodes.

The term “lower” in the context of the level of a Serpinc1 in a subject or a disease marker or symptom refers to a statistically significant decrease in such level. The decrease can be, for example, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or more and is preferably down to a level accepted as within the range of normal for an individual without such disorder.

As used herein, “prevention” or “preventing,” when used in reference to a disease, disorder or condition thereof, that would benefit from a reduction in expression of a Sertpinc1 gene, refers to a reduction in the likelihood that a subject will develop a symptom associated with a such a disease, disorder, or condition, e.g., a symptom such as a bleed. The likelihood of developing a bleed is reduced, for example, when an individual having one or more risk factors for a bleed either fails to develop a bleed or develops a bleed with less severity relative to a population having the same risk factors and not receiving treatment as described herein. The failure to develop a disease, disorder or condition, or the reduction in the development of a symptom associated with such a disease, disorder or condition (e.g., by at least about 10% on a clinically accepted scale for that disease or disorder), or the exhibition of delayed symptoms delayed (e.g., by days, weeks, months or years) is considered effective prevention.

Subjects that would benefit from a reduction and/or inhibition of Serpinc1 gene expression are those having a bleeding disorder, e.g., an inherited bleeding disorder or an acquired bleeding disorder as described herein. In one embodiment, a subject having an inherited bleeding disorder has a hemophilia, e.g., hemophilia A, B, or C. In one embodiment, a subject having an inherited bleeding disorder, e.g., a hemophilia, is an inhibitor subject (a subject that has become refractory to replacement coagulation factors). In one embodiment, the inhibitor subject has hemophilia A. In another embodiment, the inhibitor subject has hemophilia B. In yet another embodiment, the inhibitor subject has hemophilia C. Treatment of a subject that would benefit from a reduction and/or inhibition of Serpinc1 gene expression includes therapeutic (e.g., on-demand, e.g., the subject is bleeding (spontaneous bleeding or bleeding as a result of trauma) and failing to clot) and prophylactic (e.g., the subject is not bleeding and/or is to undergo surgery) treatment.

As used herein, the term “bleeding disorder” is a disease or disorder that results in poor blood clotting and/or excessive bleeding. A bleeding disorder may be an inherited disorder, such as a hemophilia or von Willebrand's disease, or an acquired disorder, associated with, for example, disseminated intravascular coagulation, pregnancy-associated eclampsia, vitamin K deficiency, an autoimmune disorder, inflammatory bowel disease, ulcerative colitis, a dermatologic disorder (e.g., psoriasis, pemphigus), a respiratory disease (e.g., asthma, chronic obstructive pulmonary disease), an allergic drug reaction, e.g., the result of medications, such as aspirin, heparin, and warfarin, diabetes, acute hepatitis B infection, acute hepatitis C infection, a malignancy or solid tumor (e.g., prostate, lung, colon, pancreas, stomach, bile duct, head and neck, cervix, breast, melanoma, kidney, and/or a hematologic malignancy). In one embodiment, an inherited bleeding disorder is a hemophilia, e.g., hemophilia A, B, or C. In one embodiment, a subject having an inherited bleeding disorder, e.g., a hemophilia, has developed inhibitors, e.g., alloantibody inhibitors, to replacement coagulation therapies and is referred to herein as an “inhibitor subject.” In one embodiment, the inhibitor subject has hemophilia A. In another embodiment, the inhibitor subject has hemophilia B. In yet another embodiment, the inhibitor subject has hemophilia C.

In one embodiment, a bleeding disorder is a rare bleeding disorder (RBD). A RBD may be an acquired RBD or an inherited RBD. Inherited RBDs include disorders associated with deficiencies of the coagulation factors fibrinogen, FII, FV, combined FV and FVIII, FVII, FX, FXI, FXIII, and congenital deficiency of vitamin K-dependent factors (VKCFDs). They are generally transmitted as autosomal recessive conditions although, in some cases, such as FXI and dysfibrinogenemia, may be autosomal dominant. RBDs are reported in most populations, with homozygous or a double heterozygous incidence varying from 1 in 500,000 for FVII deficiency to 1 in 2 to 3 million for prothrombin and FXIII deficiencies. Relative frequency varies among populations, being higher where consanguineous or endogamous marriages are common, with increased frequency of specific mutant genes.

Exemplary RBDs include afibrinogenemia (fibrinogen; Factor I deficieny); hypofibrinogenemia (fibrinogen; Factor I deficieny); dysfibrinogenemia (fibrinogen; Factor I deficieny); hypodysfibrinogenemia (fibrinogen; Factor I deficieny); hypoprothrombinemia (prothrombin; Factor II deficieny); prothrombin deficiency (prothrombin; Factor II deficieny); thrombophilia (prothrombin; Factor II deficieny); congenital antithrombin III deficiency (thromboplastin; Factor III; tissue factor); parahemophilia (proaccelerin; Factor V; labile factor); Owren's disease (proaccelerin; Factor V; labile factor); activated Protein C resistance (proaccelerin; Factor V; labile factor); Alexander's disease (stable factor proconvertin; Factor VII); congenital proconvertin/Factor VII deficiency (stable factor proconvertin; Factor VII); Stuart-Prower deficiency (Stuart-Prower factor; Factor X); congenital Factor XIIIa/b deficiency (is fibrin stabilizing factor; Factor XIII); inherited Factor XIII deficiency (fibrin stabilizing factor; Factor XIII); and fibrin stabilizing Factor deficiency (fibrin stabilizing factor; Factor XIII).

“Therapeutically effective amount,” as used herein, is intended to include the amount of an RNAi agent that, when administered to a subject having a bleeding disorder and bleeding, is sufficient to effect treatment of the disease (e.g., by diminishing, ameliorating or maintaining the existing disease or one or more symptoms of disease). The “therapeutically effective amount” may vary depending on the RNAi agent, how the agent is administered, the disease and its severity and the history, age, weight, family history, genetic makeup, the types of preceding or concomitant treatments, if any, and other individual characteristics of the subject to be treated.

“Prophylactically effective amount,” as used herein, is intended to include the amount of an iRNA that, when administered to a subject having a bleeding disorder but not bleeding, e.g., a subject having a bleeding disorder and scheduled for surgery (e.g., perioperative treatment), is sufficient to prevent or ameliorate the disease or one or more symptoms of the disease. Ameliorating the disease includes slowing the course of the disease or reducing the severity of later-developing disease. The “prophylactically effective amount” may vary depending on the iRNA, how the agent is administered, the degree of risk of disease, and the history, age, weight, family history, genetic makeup, the types of preceding or concomitant treatments, if any, and other individual characteristics of the patient to be treated.

A “therapeutically effective amount” or “prophylactically effective amount” also includes an amount of an RNAi agent that produces some desired local or systemic effect at a reasonable benefit/risk ratio applicable to any treatment. iRNA employed in the methods of the present invention may be administered in a sufficient amount to produce a reasonable benefit/risk ratio applicable to such treatment.

The “recommended therapeutically effective amount of a replacement factor” and the “recommended therapeutically effective amount of a bypassing agent” are the doses of replacement factor or bypassing agent, respectively, sufficient to generate thrombin and resolve a bleed and/or sufficient to achieve a peak level of plasma factor in a subject having a bleed as provided by the World Federation of Hemophilia (see, e.g., Srivastava, et al. “Guidelines for the Management of Hemophilia”, Hemophilia Epub 6 Jul. 2012; DOI:10.1111/j.1365-2516.2012.02909.x; ADVATE (Antihemophilic Factor (Recombinant)) product insert; November 2016; and BeneFIX (Coagulation Factor IX (Recombinant) product insert; November 2011. The entire contents of each of the forgoing are incorporated herein by reference.

For example, the recommended dose of replacement factor or bypassing agent for a subject having a minor bleed is the dose sufficient to achieve a peak plasma Factor VIII level of about 10-40 IU/dL; the recommended dose of replacement factor or bypassing agent for a subject having a moderate bleed is the dose sufficient to achieve a peak plasma Factor VIII level of about 30-60 IU/dL; the recommended dose of replacement factor or bypassing agent for a subject having a major bleed is the dose sufficient to achieve a peak plasma Factor VIII level of about 60-100 IU/dL; the recommended dose of replacement factor or bypassing agent for a subject perioperatively is the dose sufficient to achieve a peak plasma Factor VIII level of about 30-60 IU/dL (see, e.g., Tables 1 and 2 of ADVATE (Antihemophilic Factor (Recombinant)) product insert; November 2016).

The recommended dose of replacement factor or bypassing agent for a subject having a minor bleed is the dose sufficient to achieve a peak plasma Factor IX level of about 10-30 IU/dL; the recommended dose of replacement factor or bypassing agent for a subject having a moderate bleed is the dose sufficient to achieve a peak plasma Factor IX level of about 25-50 IU/dL; the recommended dose of replacement factor or bypassing agent for a subject having a major bleed is the dose sufficient to achieve a peak plasma Factor IX level of about 50-100 IU/dL.

The methods and uses of the pharmaceutical compositions of the invention generally include administering to a subject having a Serpinc1-associated disease, e.g., a bleeding disorder, e.g., a hemophilia (e.g., hemophilia A, hemophilia B, or hemophilia C), a pharmaceutical composition of the invention. In some aspects of the invention, the methods further include administering to the subject an additional therapeutic agent.

Accordingly, in one aspect, the invention provides methods of preventing at least one symptom in a subject having a disorder that would benefit from reduction in Serpinc1 expression, e.g., a bleeding disorder, e.g., a hemophilia. The methods include administering to the subject, e.g., a human, a pharmaceutical composition of the invention comprising a prophylactically effective dose, e.g., a fixed dose of about 25 mg to about 100 mg, e.g., a fixed dose of about 80 mg, of the iRNA agent, e.g., dsRNA, of the invention, thereby preventing at least one symptom in the subject having a disorder that would benefit from reduction in Serpinc1 expression.

In another aspect, the present invention provides methods of treating a subject having a disorder that would benefit from reduction in Serpinc1 expression, e.g., a bleeding disorder, e.g., a hemophilia, which include administering to the subject, e.g., a human, a pharmaceutical composition of the invention comprising a therapeutically effective dose, e.g., a fixed dose of about 25 mg to about 100 mg, e.g., a fixed dose of about 80 mg, of an iRNA agent targeting a Serpinc1 gene or a pharmaceutical composition comprising an iRNA agent targeting a Serpinc1 gene, thereby treating the subject having a disorder that would benefit from reduction in Serpinc1 expression.

In certain embodiments, the therapeutic and prophylactic methods of the invention include administering to the subject a pharmaceutical composition comprising an iRNA agent of the invention, e.g., in an amount which lowers Serpinc1 activity in the subject by about 75% or more, and a replacement factor or a bypassing agent in a therapeutically effective amount that is reduced as compared to the recommended therapeutically effective amount of the replacement factor or bypassing agent, e.g., recommended by the World Federation of Hemophilia (see, e.g., Srivastava, et al. “Guidelines for the Management of Hemophilia”, Hemophilia Epub 6 Jul. 2012; DOI:10.1111/j.1365-2516.2012.02909.x) and/or the Food and Drug Administration ((see, e.g., ADVATE (Antihemophilic Factor (Recombinant)) product insert; November 2016; BeneFIX (Coagulation Factor IX (Recombinant) product insert; November 2011) (e.g., an amount sufficient to generate thrombin and resolve a bleed (form a clot). The entire contents of each of the foregoing are incorporated herein by reference.

Suitable replacement factors include Factor VIII, e.g., Advate, Eloctate, Haemate, Helixate, Immunate, Octanate, Recombinate, and Refacto, or Factor IX, e.g., Aimafix, Benefix, Immunine, and Refacto. Suitable bypassing agents for use in the methods of the invention include activated prothrombin complex concentrates (aPCC), e.g., FEIBA and Prothromplex, and Recombinant factor VIIa (rFVIIa), e.g., NovoSeven.

The replacement factor may be Factor VIII and the therapeutically effective amount of the replacement factor administered to the subject in the methods of the invention is a dose sufficient to achieve a peak plasma Factor VIII level of about 10-100 IU/dL, e.g., about 10, 15, 20, 25, 30, 35, 40, 45, 50, 55, 60, 65, 70, 75, 80, 85, 90, 95, or about 100 IU/dL.

For example, the therapeutically effective amount of Factor VIII replacement factor administered to the subject may be less than about 200 IU/kg, or less than about 190 IU/kg, or less than about 180 IU/kg, or less than about 170 IU/kg, or less than about 160 IU/kg, or less than about 150 IU/kg, or less than about 140 IU/kg, or less than about 130 IU/kg, or less than about 120 IU/kg, or less than about 110 IU/kg, or less than about 100 IU/kg, or less than about 90 IU/kg, or less than about 80 IU/kg, or less than about 70 IU/kg, or less than about 60 IU/kg, or less than about 50 IU/kg, or less than about 40 IU/kg, or less than about 30 IU/kg, or less than about 20 IU/kg, or less than about 10 IU/kg. In one embodiment, the therapeutically effective amount of Factor VIII administered to the subject is about one and one half times to about five times less than the recommended effective amount of the replacement factor, such as a dose of about 5 to about 20 IU/kg or about 10 to about 20 IU/kg, e.g., 5, 10, 15, or 20 IU/kg. In one embodiment, the bleeding event is a moderate bleeding event. In another embodiment, the bleeding event is a major bleeding event.

The replacement factor may be Factor IX and the therapeutically effective amount of the replacement factor administered to the subject in the methods of the invention is a dose sufficient to achieve a peak plasma Factor IX level of about 10-100 IU/dL, e.g., about 10, 15, 20, 25, 30, 35, 40, 45, 50, 55, 60, 65, 70, 75, 80, 85, 90, 95, or about 100 IU/dL.

For example, the therapeutically effective amount of Factor IX replacement factor may be less than about 200 IU/kg, or less than about 190 IU/kg, or less than about 180 IU/kg, or less than about 170 IU/kg, or less than about 160 IU/kg, or less than about 150 IU/kg, or less than about 140 IU/kg, or less than about 130 IU/kg, or less than about 120 IU/kg, or less than about 110 IU/kg, or less than about 100 IU/kg, or less than about 90 IU/kg, or less than about 80 IU/kg, or less than about 70 IU/kg, or less than about 60 IU/kg, or less than about 50 IU/kg, or less than about 40 IU/kg, or less than about 30 IU/kg, or less than about 20 IU/kg, or less than about 10 IU/kg. In one embodiment, the therapeutically effective amount of Factor IX administered to the subject is about two times to about six times less than the recommended effective amount of the replacement factor, e.g., a dose of about 10 to about 30 IU/kg or about 20 to about 30 IU/kg, such as, about 10, 15, 20, 25, or 30 IU/kg. In one embodiment, the bleeding event is a moderate bleeding event. In another embodiment, the bleeding event is a major bleeding event

The bypassing agent may be aPCC and the therapeutically effective amount of the bypassing agent administered to the subject in the methods of the invention is a dose sufficient to generate thrombin and resolve a bleed.

For example, the therapeutically effective amount of the bypassing agent aPCC may be less than about 100 U/kg, or less than about 90 U/kg, or less than about 80 U/kg, or less than about 70 U/kg, or less than about 60 U/kg, or less than about 50 U/kg, or less than about 40 U/kg, or less than about 30 U/kg, or less than about 20 U/kg, or less than about 10 U/kg. In one embodiment, the therapeutically effective amount of aPCC administered to the subject is about two times to about three times less than the recommended effective amount of the replacement factor, e.g., a dose of about 30 to about 50 U/kg, such as, about 30, 35, 40, 45, or 50 U/kg. In one embodiment, the bleeding event is a moderate bleeding event. In another embodiment, the bleeding event is a major bleeding event.

The bypassing agent may be rFVIIa and the therapeutically effective amount of the bypassing agent administered to the subject in the methods of the invention is a dose sufficient to generate thrombin and resolve a bleed.

For example, the therapeutically effective amount of the bypassing agent rFVIIa is less than about 120 μg/kg, or less than about 110 μg/kg, or less than about 100 μg/kg, or less than about 90 μg/kg, or less than about 80 μg/kg, or less than about 70 μg/kg, or less than about 60 μg/kg, or less than about 50 μg/kg, or less than about 40 μg/kg, or less than about 30 μg/kg, or less than about 20 μg/kg. In one embodiment, the therapeutically effective amount of rFVIIa administered to the subject is about two times less than the recommended effective amount of the replacement factor, e.g., a dose of about 45 μg/kg. In one embodiment, the bleeding event is a moderate bleeding event. In another embodiment, the bleeding event is a major bleeding event.

In some embodiment, a pharmaceutical composition comprising the dsRNA agent is administered to a subject at a fixed dose. A “fixed dose” (e.g., a dose in mg) means that one dose of an iRNA agent is used for all subjects regardless of any specific subject-related factors, such as weight. In one particular embodiment, a fixed dose of an iRNA agent of the invention is based on a predetermined weight or age.

In some embodiments, the pharmaceutical composition comprising the iRNA agent is administered at a fixed dose of between about 25 mg to about 100 mg, e.g., between about 25 mg to about 95 mg, between about 25 mg to about 90 mg, between about 25 mg to about 85 mg, between about 25 mg to about 80 mg, between about 25 mg to about 75 mg, between about 25 mg to about 70 mg, between about 25 mg to about 65 mg, between about 25 mg to about 60 mg, between about 25 mg to about 50 mg, between about 50 mg to about 100 mg, between about 50 mg to about 95 mg, between about 50 mg to about 90 mg, between about 50 mg to about 85 mg, between about 50 mg to about 80 mg, between about 30 mg to about 100 mg, between about 30 mg to about 90 mg, between about 30 mg to about 80 mg, between about 40 mg to about 100 mg, between about 40 mg to about 90 mg, between about 40 mg to about 80 mg, between about 60 mg to about 100 mg, between about 60 mg to about 90 mg, between about 25 mg to about 55 mg, between about 30 mg to about 95 mg, between about 30 mg to about 85 mg, between about 30 mg to about 75 mg, between about 30 mg to about 65 mg, between about 30 mg to about 55 mg, between about 40 mg to about 95 mg, between about 40 mg to about 85 mg, between about 40 mg to about 75 mg, between about 40 mg to about 65 mg, between about 40 mg to about 55 mg, or between about 45 mg to about 95 mg.

In some embodiments, the pharmaceutical composition comprising the iRNA agent is administered at a fixed dose of about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg.

In one embodiment, the RNAi agent is administered to the subject at a fixed dose of about 100 mg.

In one embodiment, the RNAi agent is administered to the subject at a dose which lowers Serpinc1 activity by about 75% or more

A pharmaceutical composition comprising the iRNA agent may be administered to a subject as one or more doses.

A pharmaceutical composition comprising the iRNA may be administered to the subject about once a month, about once every five weeks, about once every six weeks, about once every 2 months, or once a quarter.

In some embodiments, a single dose of the pharmaceutical compositions can be long lasting, such that subsequent doses are administered at not more than 1, 2, 3, 4, 5, 6, 7, or 8 week intervals. In some embodiments of the invention, a single dose of the pharmaceutical compositions of the invention is administered once per month. In one embodiment, the fixed dose of the RNAi agent is suitable for administration to the subject once a month, such as a fixed dose of 80 mg once per month.

The methods and uses of the invention include administering a composition described herein such that expression of the target Serpinc1 gene is decreased, such as for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or about 80 days. In one embodiment, expression of the target Serpinc1 gene is decreased for an extended duration, e.g., at least about seven days or more, e.g., about one week, two weeks, three weeks, about four weeks, about 5 weeks, about 6 weeks, about 2 months, about a quarter, or longer.

Reduction in gene expression can be assessed by any methods known in the art. For example, a reduction in the expression of Serpinc1 may be determined by determining the mRNA expression level of Serpinc1 using methods routine to one of ordinary skill in the art, e.g., Northern blotting, qRT-PCR, by determining the protein level of Serpinc1 using methods routine to one of ordinary skill in the art, such as Western blotting, immunological techniques, and/or by determining a biological activity of Serpinc1, such as affecting one or more molecules associated with the cellular blood clotting mechanism (or in an in vivo setting, blood clotting itself). In one embodiment, thrombin generation time, clot formation time and/or clotting time are determined to assess Serpinc1 expression using, e.g., ROTEM® Thromboelastometry analysis of whole blood.

Administration of the dsRNA according to the methods and uses of the invention may result in a reduction of the severity, signs, symptoms, and/or markers of such diseases or disorders in a patient with a Serpinc1-associated disease. By “reduction” in this context is meant a statistically significant decrease in such level. The reduction can be, for example, at least about 5%, 10%, 15%, 20%, 25%, 30%, 35%, 40%, 45%, 50%, 55%, 60%, 65%, 70%, 75%, 80%, 85%, 90%, 95%, or about 100%.

Efficacy of treatment or prevention of disease can be assessed, for example by measuring disease progression, disease remission, symptom severity, frequency of bleeds, reduction in pain, quality of life, dose of a medication required to sustain a treatment effect, level of a disease marker or any other measurable parameter appropriate for a given disease being treated or targeted for prevention. It is well within the ability of one skilled in the art to monitor efficacy of treatment or prevention by measuring any one of such parameters, or any combination of parameters. For example, efficacy of treatment of a bleeding disorder may be assessed, for example, by periodic monitoring of thrombin:anti-thrombin levels. Comparisons of the later readings with the initial readings provide a physician an indication of whether the treatment is effective. It is well within the ability of one skilled in the art to monitor efficacy of treatment or prevention by measuring any one of such parameters, or any combination of parameters. In connection with the administration of an iRNA targeting Serpinc1 or pharmaceutical composition thereof, “effective against” a bleeding disorder indicates that administration in a clinically appropriate manner results in a beneficial effect for at least a statistically significant fraction of patients, such as a improvement of symptoms, a cure, a reduction in disease, extension of life, improvement in quality of life, or other effect generally recognized as positive by medical doctors familiar with treating bleeding disorders and the related causes.

A treatment or preventive effect is evident when there is a statistically significant improvement in one or more parameters of disease status, or by a failure to worsen or to develop symptoms where they would otherwise be anticipated. As an example, a favorable change of at least 10% in a measurable parameter of disease, and preferably at least 20%, 30%, 40%, 50% or more can be indicative of effective treatment. Efficacy for a given iRNA drug or formulation of that drug can also be judged using an experimental animal model for the given disease as known in the art. When using an experimental animal model, efficacy of treatment is evidenced when a statistically significant reduction in a marker or symptom is observed.

Alternatively, the efficacy can be measured by a reduction in the severity of disease as determined by one skilled in the art of diagnosis based on a clinically accepted disease severity grading scale. Any positive change resulting in e.g., lessening of severity of disease measured using the appropriate scale, represents adequate treatment using an iRNA or iRNA formulation as described herein.

The invention further provides methods and uses for the use of an iRNA or a pharmaceutical composition thereof, e.g., for treating a subject that would benefit from reduction and/or inhibition of Serpinc1 expression, e.g., a subject having a bleeding disorder, in combination with other pharmaceuticals and/or other therapeutic methods, e.g., with known pharmaceuticals and/or known therapeutic methods, such as, for example, those which are currently employed for treating these disorders.

For example, in certain embodiments, an iRNA targeting Serpinc1 is administered in combination with, e.g., an agent useful in treating a bleeding disorder as described elsewhere herein. For example, additional therapeutics and therapeutic methods suitable for treating a subject that would benefit from reduction in Serpinc1 expression, e.g., a subject having a bleeding disorder, include fresh-frozen plasma (FFP); recombinant FVIIa; recombinant FIX; FXI concentrates; virus-inactivated, vWF-containing FVIII concentrates; desensitization therapy which may include large doses of FVIII or FIX, along with steroids or intravenous immunoglobulin (IVIG) and cyclophosphamide; plasmapheresis in conjunction with immunosuppression and infusion of FVIII or FIX, with or without antifibrinolytic therapy; immune tolerance induction (ITI), with or without immunosuppressive therapy (e.g., cyclophosphamide, prednisone, and/or anti-CD20); desmopressin acetate [DDAVP]; antifibrinolytics, such as aminocaproic acid and tranexamic acid; activated prothrombin complex concentrate (PCC); antihemophilic agents; corticosteroids; immunosuppressive agents; and estrogens.

The iRNA and an additional therapeutic agent and/or treatment may be administered at the same time and/or in the same combination, e.g., parenterally, or the additional therapeutic agent can be administered as part of a separate composition or at separate times and/or by another method known in the art or described herein.

### VI. Containers of the Invention

The present invention also provides containers, such a vials, syringes, autoinjector pens, or needle-free administration devices, comprising a pharmaceutical composition of the invention.

In one embodiment, the compositions of the invention may be used for self administration using, e.g., a preloaded syringe or an automatic injection device.

In one embodiment, a container comprising a pharmaceutical composition of the invention is a vial. The vial may include about 0.5 mL to about 2.0 ml of the pharmaceutical composition.

In one embodiment, the vial comprises about 0.8 ml of the pharmaceutical composition. In one embodiment, the vial is a 2R vial (i.e., a 2 ml injection vial) comprising a single dose of the pharmaceutical composition. In one embodiment, the 2R vial comprises about 0.80 ml (e.g., about 0.96 to about 1.05 mL) of a pharmaceutical composition of the invention comprising a single 80 mg dose of the composition.

In one embodiment, a container of the invention comprises a syringe, such as a pre-filled syringe. In one embodiment, the pre-filled syringe includes a needle sharp injury prevention safety feature (PFS-S). Suitable syringes may be 1 ml syringes or 3 ml syringes and include a 29 G needle or a 30 G needle. In one embodiment, the syringe is a single-use 3 ml glass syringe with a 29 G or 30 G needle. In one embodiment, the pre-filled syringe comprises about 0.80 ml (e.g., about 0.84 ml, or 0.8 to 0.84 ml) of a pharmaceutical composition of the invention comprising a single 80 mg dose of the composition.

An exemplary pre-filled syringe of the invention may include a syringe, such as a BD Neopak with 29 G X ½″ needle; rigid needle shield (RNS); a plunger, such as a BD 4023 plunger with FluroTec coating; a safety system, such as a BD UltraSafelm Plus; a plunger rod, such as a BD UltraSafe' Passive Plunger Rod; and a finger flange, such as a BD UltraSaferm Passive Add-on Finger.

### VII. Kits of the Invention

The present invention also provides kits comprising a pharmaceutical composition. Such kits include one or more vials or one or more pre-filled syringes comprising a pharmaceutical composition of the invention and instructions for use, e.g., instructions for administering a prophylactically or therapeutically effective amount of an RNAi agent(s). The kits may optionally further comprise means for administering the RNAi agent (e.g., an injection device), or means for measuring the inhibition of Serpinc1 (e.g., means for measuring the inhibition of Serpinc1 mRNA, Serpinc1 protein, and/or Serpinc1 activity). Such means for measuring the inhibition of Serpinc1 may comprise a means for obtaining a sample from a subject, such as, e.g., a plasma sample. The kits of the invention may optionally further comprise means for determining the therapeutically effective or prophylactically effective amount.

Unless otherwise defined, all technical and scientific terms used herein have the same meaning as commonly understood by one of ordinary skill in the art to which this invention belongs. Although methods and materials similar or equivalent to those described herein can be used in the practice or testing of the iRNAs and methods featured in the invention, suitable methods and materials are described below. All publications, patent applications, patents, and other references mentioned herein are incorporated herein by reference in their entirety. In addition, the materials, methods, and examples are illustrative only and not intended to be limiting.

This invention is further illustrated by the following examples which should not be construed as limiting. The contents of all references, patents and published patent applications cited throughout this application, as well as the Figures, are hereby incorporated by reference.

### Examples

TABLE 1
Abbreviations of nucleotide monomers used in nucleic acid sequence
representation. It will be understood that these monomers, when present
in an oligonucleotide, are mutually linked by 5′-3′-phosphodiester bonds.

| Abbreviation | Nucleotide(s) |
|---|---|
| A | Adenosine-3′-phosphate |
| Af | 2′-fluoroadenosine-3′-phosphate |
| Afs | 2′-fluoroadenosine-3′-phosphorothioate |
| As | adenosine-3′-phosphorothioate |
| C | cytidine-3′-phosphate |
| Cf | 2′-fluorocytidine-3′-phosphate |
| Cfs | 2′-fluorocytidine-3′-phosphorothioate |
| Cs | cytidine-3′-phosphorothioate |
| G | guanosine-3′-phosphate |
| Gf | 2′-fluoroguanosine-3′-phosphate |
| Gfs | 2′-fluoroguanosine-3′-phosphorothioate |
| Gs | guanosine-3′-phosphorothioate |
| T | 5′-methyluridine-3′-phosphate |
| Tf | 2′-fluoro-5-methyluridine-3′-phosphate |
| Tfs | 2′-fluoro-5-methyluridine-3′-phosphorothioate |
| Ts | 5-methyluridine-3′-phosphorothioate |
| U | Uridine-3′-phosphate |
| Uf | 2′-fluorouridine-3′-phosphate |
| Ufs | 2′-fluorouridine-3′-phosphorothioate |
| Us | uridine-3′-phosphorothioate |
| N | any nucleotide (G, A, C, T or U) |
| a | 2′-O-methyladenosine-3′-phosphate |
| as | 2′-O-methyladenosine-3′-phosphorothioate |
| c | 2′-O-methylcytidine-3′-phosphate |
| cs | 2′-O-methylcytidine-3′-phosphorothioate |
| g | 2′-O-methylguanosine-3′-phosphate |
| gs | 2′-O-methylguanosine-3′-phosphorothioate |
| t | 2′-O-methyl-5-methyluridine-3′-phosphate |
| ts | 2′-O-methyl-5-methyluridine-3′-phosphorothioate |
| u | 2′-O-methyluridine-3′-phosphate |
| us | 2′-O-methyluridine-3′-phosphorothioate |
| s | phosphorothioate linkage |
| L96 | N-[tris(GalNAc-alkyl)-amidodecanoyl)]-4-hydroxyprolinol |
| | Hyp-(GalNAc-alkyl)3 |

### Example 1: Fitusiran Drug Product

Fitusiran drug product (Fitusiran) is a sterile solution containing 100 mg/mL fitusiran (equivalent to 106 mg/mL fitusiran sodium) in 5 mM phosphate buffered saline (PBS) for subcutaneous administration. The drug product is commercially supplied as a 0.8 mL solution in 2R Type I glass vial with teflon coated butyl-rubber stopper and center tear over seals. The drug product does not contain preservatives and is intended for single use.

The composition of fitusiran drug product is summarized in Table 2.

TABLE 2
Composition of Fitusiran Drug Product

| Component, | Concentration | Per vial | |
|---|---|---|---|
| grade | (mg/mL) | (mg)^{a} | Function |
| fitusiran (cGMP) | 100 | 80 | Active |
| PBS: | | | Ingredient |
| Monosodium Phosphate | 0.0885 | 0.0708 | Buffer |
| Monohydrate (BP, USP) | | | Salt |
| Disodium Phosphate | 1.169 | 0.9352 | Buffer |
| Heptahydrate (USP) | | | Salt |
| Sodium Chloride | 4.909 | 3.9272 | Buffer |
| (Ph. Eur., BP, JP, USP) | | | Salt |
| Water for Injection | q.s. | q.s. | Diluent |
| (Ph. Eur., USP) | | | |

^{a}Sodium hydroxide (USP, Ph. Eur.) and phosphoric acid (USP, Ph. Eur.) are used only if pH adjustment is needed for a particular batch.
Abbreviations:
PBS = Phosphate Buffered solution;
BP = British Pharmacopeia;
Ph.Eur. = European Pharmacopeia;
USP = United States Pharmacopeia;
q.s. = quantity sufficient

The chemical structure of Fitusiran, shown below, is represented using an expanded structural formula showing the phosphate backbone. The bases involved in base pair formation are connected with a dotted line. The structure of L96, the GalNAc containing ligand, and the linker conjugating the ligand to the 3′-end of the sense strand is also presented below. The molecular formulas and masses of the duplex and the two single strands (AD-116858, sense strand; A-116861, antisense strand) of the Fitusiran duplex (AD-57213) are also provided in the Table below.

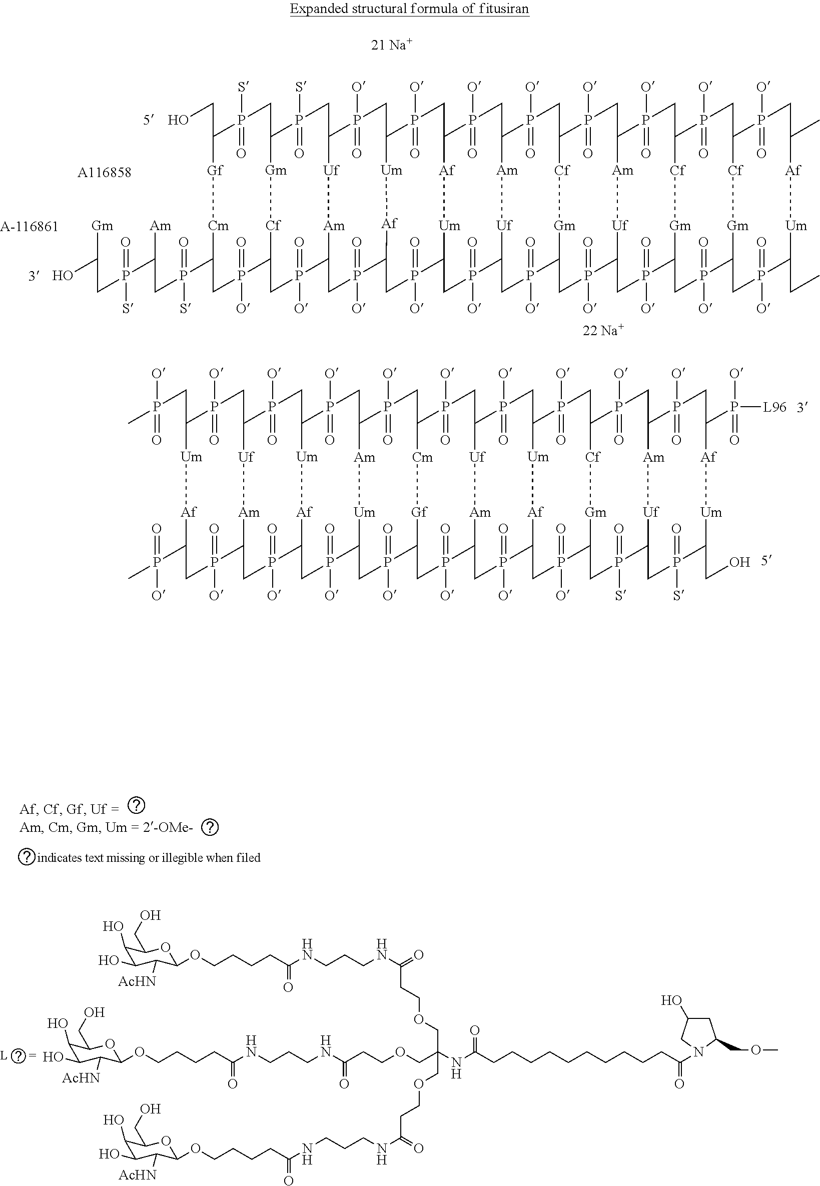

Molecular formula and molecular mass

| | Fitusiran | A-116858 | A-116861 |
|---|---|---|---|
| | (Duplex) | (Sense strand) | (Antisense strand) |
| Molecular formula | [symbol] | [symbol] | [symbol] |
| sodium salt | | | |
| Molecular formula | [symbol] | [symbol] | [symbol] |
| free acid | | | |
| Molecular weight | 17,193 Da | 9,035 Da | 8,159 Da |
| sodium salt | | | |
| Molecular weight | 16,248 Da | 8,573 Da | 7,675 Da |
| free acid | | | |

[symbol]

indicates data missing or illegible when filed

The fitusiran formulation was designed for subcutaneous administration. Formulations designed for subcutaneous administration should not be too acidic or too alkaline to avoid the risk of increased irritation and chemical incompatibility. With due consideration of tonicity, pH, and viscosity, the formulation was designed to be as close to physiological as possible. The pH of aqueous solutions of fitusiran drug product at 100 mg/mL varies from 5.0 to 6.8. The presence of sodium counter ions with anionic phosphodiester contributes a certain amount of osmolality which is dependent on the concentration of the aqueous solution. At the target formulation of 100 mg/mL drug substance, the counter ions gives rise to an approximately 118 mOsm/kg solution. To maintain the isotonicity and buffer capacity of the drug product, drug substance was dissolved in a 5 mM phosphate buffered saline (0.64 mM NaH2PO4, 4.36 mM Na2HPO4, 84 mM NaCl).

The drug product formulation described above has the following physicochemical properties: pH of about 6.8 to about 7.2; Osmolality of about 300 mOsm/kg; and a Density of about 1.038 g/mL.

Fitusiran drug product manufacturing consisted of dissolving the required amount of the powdered (lyophilized) fitusiran drug substance in 5 mM phosphate buffered saline and adjusting the pH with sodium hydroxide or phosphoric acid to approximately 7.0, followed by sterile filtration and filling.

The drug product used in early development, including nonclinical and Phase ½ clinical studies, was supplied as a 100 mg/mL (fitusiran sodium) solution in a nominal 0.5 mL per vial. The drug product which is used in the Phase 3 study and intended for commercial production is supplied as a 100 mg/mL (fitusiran free acid, equivalent to 106 mg/mL fitusiran sodium) in a nominal 0.8 mL per vial. The table below summarizes the differences in Fitusiran drug product formulations.

| | Manufacturing Scale | Concentration | Nominal Fill Volume |
|---|---|---|---|
| Drug Product Batch | [L] | [mg/mL] | [mL] |
| Development and | About 1.5 | 100 (fitusiran sodium) | 0.5 |
| clinical lots | | equivalent to 94 | |
| (Phase 1 and 2) | | (fitusiran free acid) | |
| Clinical lots for Phase 3 | About 1.5 | 100 (fitusiran free acid) | 0.8 |
| (manufacturer AMRI) | | equivalent to 106 | |
| | | (fitusiran sodium) | |
| Clinical lots for Phase 3 | About 5.0 | 100 (fitusiran free acid) | 0.8 |
| (manufacturer Vetter | | equivalent to 106 | |
| Pharma) | | (fitusiran sodium) | |

### Example 3: Analytical Analyses of Fitusiran Drug Product

Various analytical evaluations of the Fitusiran drug product were performed to demonstrate that the drug product was physically and chemically stable.

### Appearance

The fitusiran drug product was visually examined for color, homogeneity and particulate matter against a black and white background under diffuse uniform illumination.

Appearance testing for fitusiran drug product was performed with the same visual test throughout different batches and the results met the specification of “clear, colorless to pale yellow solution essentially free of particulates.”

### Identity by Duplex Retention Time

The fitusiran drug product was analyzed by non-denaturing IP RP-HPLC together with a fitusiran reference standard and the duplex retention time of the sample was compared to that of the reference standard. All fitusiran drug product batches manufactured to date met the specification of “retention time consistent with that of the reference standard,” confirming their identity as annealed siRNA duplexes.

### Assay of Fitusiran Drug Product by UV

UV absorbance method was used for the determination of assay (mg/mL) of fitusiran in the fitusiran drug product. The absorbance of a suitably diluted drug product in 0.9% saline is measured with a UV spectrophotometer at 260 nm.

C=(A×F×M)/(ε×b),

where A is the measured absorbance, F is the dilution factor, b is the path length of the cell (1 cm), ε is molar absorptivity of duplex reference standard, M is the molecular weight and C is the concentration (mg/mL). To account for duplex purity, the fitusiran assay result for a given drug product is corrected for its purity factor (multiplied by (non-denaturing IP-RP HPLC area-%)/100).

The Assay of the fitusiran drug product (mg/mL) was determined from UV spectrophotometry and corrected for duplex purity from the non-denaturing IP RP-HPLC method and the results are reported on the basis of the concentration of the H-form (free acid) of the duplex. All results were within the specification limits of 90 to 110 mg/mL (measured as free acid form), with a mean assay value of 101.5 mg/mL and a standard deviation of 3.4%. The results showed good comparability between the assay values of all fitusiran lots tested.

### pH of Fitusiran Drug Product

The pH of the fitusiran drug product was measured directly. The comparative pH results for the fitusiran drug product lots were observed to be pH of 7.1 with a standard deviation of 0.0. All of the results met the current specification of 6.0-8.0 for pH for fitusiran drug product. Analysis of the pH data for the fitusiran drug product batches indicated a high degree of comparability between the fitusiran lots.

### Osmolality of Fitusiran Drug Product

The osmolality of fitusiran drug product is based on principle of freezing-point depression. Osmolality was reported as mOsm/kg value. Since the formulation has fixed salt concentrations from the sodium phosphate buffer and fitusiran duplex, the observed osmolality values showed only a narrow range. Osmolality results for the fitusiran drug product batches ranged from 297-310 (mOsm/kg), a mean of 304 mOsm/kg and a standard deviation of 5.3%. All results were within the specification of 240-390 mOsm/kg for osmolality of fitusiran drug product.

### Particulate Matter in Fitusiran Drug Product

Fitusiran drug product was analyzed for number of sub-visible particulate matter per container by light obscuration method and the results were reported in total number of particles (≥10 μm and ≥25 μm) per container. For particles≥10 μm in fitusiran drug product, the observed range was about 29-588 particles (≥10 μm), a mean of about 188 particles and a standard deviation of 268.2%. All results were within the specification of NMT 6,000 per container of fitusiran drug product.

For particles≥25 μm, the observed range was about 0-46 particles, a mean of about 13 particles and a standard deviation of 22.4%. All results were within the specification of NMT 600 per container of fitusiran drug product.

### Volume in Container for Fitusiran Drug Product

The volume in containers comprising the fitusiran drug product was measured with a specification limit set to not less than (NLT) 0.8 mL. The volume in containers observed in the different fitusiran drug product lots showed a good degree of comparability between the fitusiran drug product batches with standard deviation of 0.0.

### Bacterial Endotoxins and Sterility in Fitusiran Drug Product Batches

All batches of fitusiran drug product met the microbial safety testing criteria for bacterial endotoxins (not more than (NMT) 100 endotoxin units (EU)/mL), demonstrating proper control for Fitusiran drug product process and its microbial safety profile.

### Duplex Purity by Non-Denaturing Ion-Pair Reversed-Phase High Performance Liquid Chromatography (IP RP-HPLC)

Non-denaturing IP RP-HPLC resolves the duplex from any residual single strand. The area percent purity of the duplex is determined by this method. The identity of the drug substance in fitusiran drug product was established by retention time consistent with that of duplex reference standard.

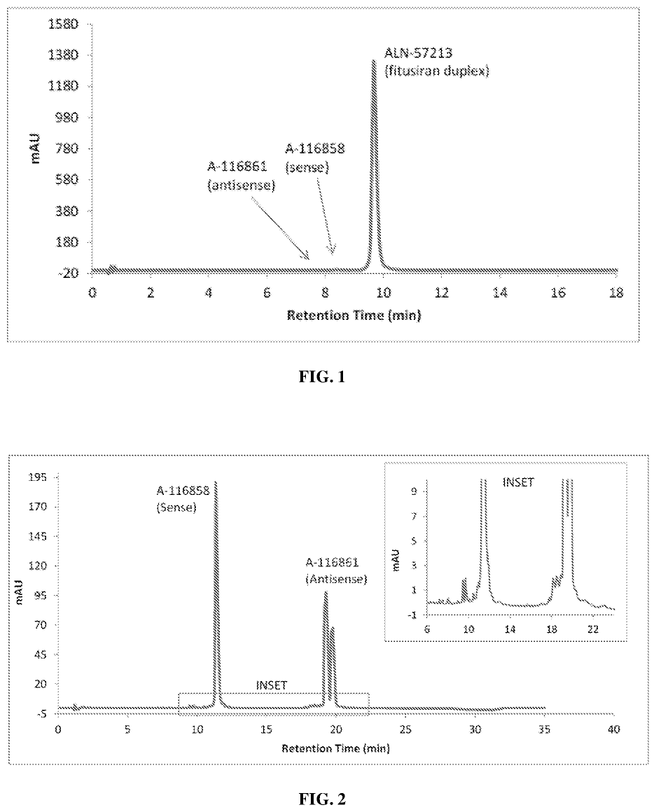

Non-denaturing IPRP HPLC method was used for identification of the constituent single strands, sense and antisense strands in the drug product in tandem with mass spectrometry (ESI-MS). As duplex peak was resolved from the residual single strand, duplex purity was determined by this method. A representative IP RP chromatogram of fitusiran drug product is shown in FIG. 1.

Stationary phase: Waters XBridge C8 2.1×50 column, 2.5 μm particle size.

Mobile phase A: 95 mM 1,1,1,3,3,3-hexafluoro-2-propanol (HFIP), 16 mM triethylamine (TEA), 5 μM ethylenediaminetetraacetic acid (EDTA) in water.

Mobile phase B: 100% methanol with 5 μM EDTA.

Flow rate: 0.25 mL/min.

Column temperature: 15° C.

Gradient:

| Time (min) | % A | % B |
|---|---|---|
| Initial | 80 | 20 |
| 1.00 | 80 | 20 |
| 15.25 | 35 | 65 |
| 15.75 | 20 | 80 |
| 16.75 | 20 | 80 |
| 17.00 | 80 | 20 |
| 24.00 | 80 | 20 |

Detection: UV at 260 nm and MS in negative ion mode for 700 to 2700 Da

Sample preparation: Sample was prepared in 1×PBS to a concentration of ˜0.1 mg/mL for single strand intermediates and 0.2 mg/mL for duplex drug substance (fitusiran).

Injection volume is 20 μL.

Limits of detection: Chromatography Software was used to integrate and report all peaks ≥ to 0.05 area %.

Purity calculation: The area % of the main duplex peak was calculated by chromatography software and reported as duplex purity. Area-% of residual single strand and other impurities were reported as well.

Identity: The identity of the constituent single strands, sense and antisense present under the same duplex peak, was established by molecular weight determined from the deconvolution of the duplex peak spectra by liquid chromatography mass spectrometry (LC-MS) using Chromatographic software.

The non-denaturing profile of the fitusiran drug product by non-denaturing IP RP-HPLC confirmed the presence of the drug product in its duplex form. Duplex purity indicated the percentage of annealed duplex siRNA in fitusiran drug product. For the drug product batches included in this study, the duplex purity values were in the range of about 98.9-99.5 area %. Analysis of the data yielded a mean (n=4) purity of about 99.2% with a standard deviation of about 0.3% and showed that the duplex purity values were consistent and similar to each other for all of the fitusiran drug product batches compared in this report.

### Total Impurities by Non-Denaturing Ion-Pair Reversed-Phase High Performance Liquid Chromatography (IP RP-HPLC)

The non-duplex (non-annealed) impurities by non-denaturing IP RP-HPLC were reported as the sum of all (non-duplex) peaks ≥0.050 area %. The results for the total impurities by the non-denaturing IP RP-HPLC were observed to be within 2% and met specification of NMT 10.0 area % for all batches of fitusiran drug product included in this study. The mean value (n=4) for the total impurities by the non-denaturing IP RP-HPLC was 0.85% with a standard deviation of 0.3%. Overall, the results showed that the fitusiran drug product lots examined in this report had very similar profiles in terms of both specified (single strands) and non-specified impurities.

### Purity by Denaturing Anion Exchange High Performance Liquid Chromatography (AX-HPLC)

In order to determine the purity of the single strands in the drug product, denaturing AX-HPLC analysis was performed.

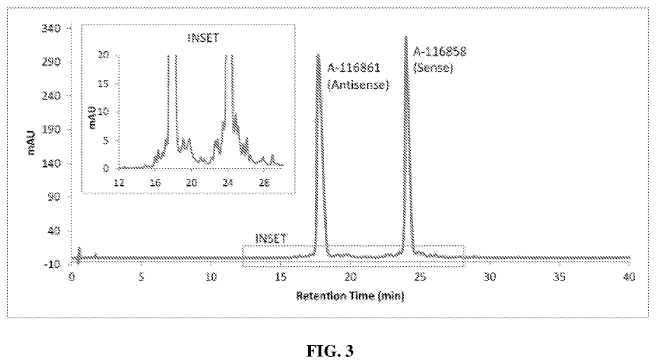

The presence of multiple peaks for the antisense strand was due to the phosphorothioate diastereomers. A representative AX-HPLC chromatogram of fitusiran drug product is shown in FIG. 2.

Stationary phase: Dionex DNA Pac PA200 column, 4×250 mm

Mobile phase A: 20 mM Sodium Phosphate, 10% ACN, pH 11

Mobile phase B: 20 mM Sodium Phosphate, 1M NaBr, 10% ACN, pH 11

Gradient:

| Time (min) | % A | % B |
|---|---|---|
| Initial | 65 | 35 |
| 26.00 | 36.5 | 63.5 |
| 27.00 | 0 | 100 |
| 29.00 | 0 | 100 |
| 30.00 | 65 | 35 |
| 35.00 | 65 | 35 |

Limits of detection: Chromatography Software was used to integrate and report all peaks ≥ to 0.05 area %

Purity calculation: The area % of each of the main peaks (sense and antisense) was calculated by chromatography software and sum of sense and antisense strands area % was reported as purity. Sum of % area of impurities above 0.050 area % was reported as total impurities.

Identity: Single strand identity was confirmed by comparing the retention times of the test sample with that of the corresponding reference standard

AX-HPLC denatures the Fitusiran duplex to form the constituent sense and antisense single strands. The area-percent purity of the single strands was determined by this method.

The denaturing AX-HPLC method measures the purity of the individual single strands comprising the fitusiran duplex. The sum of the single strands area percentages represents the denaturing purity of the fitusiran drug product. Analysis of the sum of the area % of the sense and the antisense strands for the fitusiran lots in this study yielded a mean value (n=4) of 94.2 area % with a standard deviation of 0.8%. All the results from the representative fitusiran drug product batches met the specification of NLT 85.0 area % and indicated overall comparable purity results for fitusiran drug product.

### Total Impurities by Denaturing Anion Exchange High Performance Liquid Chromatography (AX-HPLC)

Analysis of the data yields a mean value (n=4) of 5.6% for the sum of all impurities NLT 0.050 area % with a standard deviation of 0.7%. The results show that the total impurity value for the fitusiran batches included in this report is consistent and comparable.

### Purity by Denaturing Ion-Pair Reversed-Phase High Performance Liquid Chromatography (IP RP-HPLC)

IP RP-HPLC denatures the Fitusiran duplex to form the constituent sense and antisense single strands. The area-percent purity of the single strands is determined by this method.

The denaturing IP RP-HPLC method is orthogonal to the AX-HPLC and measures the purity of the individual single strands comprising the fitusiran duplex in the drug product. The sum of the single strands area percentages represents the denaturing IP RP-HPLC purity of the fitusiran drug product.

Denaturing IP RP-HPLC analysis was also performed to determine the purity of the single strands in the drug product.

Stationary phase: Waters XBridge C18 (OST or XP) 2.1×50 column, 2.5 μm particle size.

Mobile phase A: 550 mM 1,1,1,3,3,3-hexafluoro-2-propanol (HFIP), 13 mM trimethylamine (TEA), and 5 M ethylenediaminetetraacetic acid (EDTA) in 90:10 water:methanol.

Mobile phase B: 100% methanol

Flow rate: 0.40 mL/min.

Column temperature: 80° C.

Gradient:

| Time (min) | % A | % B |
|---|---|---|
| Initial | 94 | 6 |
| 2.0 | 94 | 6 |
| 35.0 | 82.5 | 17.5 |
| 40.0 | 56.5 | 43.5 |
| 40.5 | 30 | 70 |
| 42.5 | 30 | 70 |
| 43.0 | 94 | 6 |
| 49.0 | 94 | 6 |

Detection: UV at 260 nm and MS in negative ion mode for 700 to 2700 Da Sample preparation: Sample was prepared in 1×PBS to a concentration of ˜0.1 mg/mL for single strand intermediates and 0.2 mg/mL for duplex drug substance (fitusiran).

Injection volume was 25 μL.

Limits of detection: Chromatography Software was used to integrate and report all peaks ≥ to 0.05 area %.

Purity calculation: The area % of the main duplex peak was calculated by chromatography software and reported as duplex purity. Area-% of residual single strand and other impurities were reported as well.

A representative denaturing IP RP-HPLC chromatographic profile of fitusiran drug product is shown in FIG. 3.

Analysis of the sum of the area % of the sense and the antisense strands for the fitusiran lots in this study yielded a mean value (n=4) of 88.7 area % with a standard deviation of 1.4%. All the results for the fitusiran drug product lots met the specification of NLT 80.0 area % and showed, within analytical variability, a comparable purity results for fitusiran drug product lots.

### Total Impurities by Denaturing Ion-Pair Reversed-Phase High Performance Liquid Chromatography (IP RP-HPLC)

Analysis of the data yields a mean value (n=4) of 11.0% for the sum of all impurities NLT 0.050 area % with a standard deviation of 1.5%. The results show that the total impurity value for all the fitusiran batches included in this report is consistent and comparable.

### Example 4: Container Closure System and Compatibility of Fitusiran Drug Product

The container closure system for fitusiran drug product was chosen to protect the sterile product from microbiological contamination. Vials are sterilized and depyrogenated by dry heat at ≥300° C. for ≥5 minutes. Butyl-rubber seals are autoclaved at 121-125° C. for ≥60 minutes. Butyl-rubber stoppers are steam sterilized by autoclave through a validated cycle. All components are standard items for parenteral products. The fitusiran drug product stability studies are conducted using the drug product stored in an identical container closure system.

Fitusiran is formulated for subcutaneous injection. Based on the estimated calculated doses to be administered, 1 mL or 3 mL syringes will be used. Two syringe types, one with polycarbonate material of construction and the second with polypropylene material of construction, were tested for compatibility with fitusiran. The drug product, 100 mg/mL filled in the vials, was drawn into the syringes. One set of filled syringes was incubated at 25° C. for 8 h and another set of filled syringes was incubated at 2-8° C. for 48 h together with controls. After the incubation, the drug product was tested for assay and purity by AX-HPLC and compared to vialed drug product. There was no difference among control drug product and drug product incubated in the two syringe types in terms of label claim and purity, indicating compatibility of fitusiran with the intended injection devices as shown below in Table 3.

TABLE 3
Compatibility Data

| | | | | Assay | Purity |
|---|---|---|---|---|---|
| | Material of | Incubation | Temperature | (mg/ | AX-HPLC |
| Description | Construction | Time (hr) | (° C.) | mL)¹ | (area %) |
| Control | NA | T0 | 2-8 | 99.2 | 89.6 |
| in vial | | | | | |
| 1 mL syringe | polycarbonate | 8 | 25 | 100 | 89.8 |
| 3 mL syringe | polycarbonate | 8 | 25 | 99.7 | 89.6 |
| 1 mL syringe | polycarbonate | 48 | 2-8 | 98.2 | 89.8 |
| 3 mL syringe | polycarbonate | 48 | 2-8 | 98.7 | 89.7 |
| Control | NA | 48 | 2-8 | 99.4 | 89.7 |
| in vial | | | | | |

The use of larger gauge (narrower bore) needles for both extracting and dispensing undiluted drug product, with the syringes of the same materials of constructions as shown in Table 3, was also evaluated. Two syringe types and two rates of dispensing were analyzed. The results are shown in Table 4 and demonstrate that that the integrity of the fitusiran drug product remains intact when used with 29 or 30 G needles.

TABLE 4
Additional Compatibility Data

| | | Non-denaturing IP RP- | | AX-HPLC |
|---|---|---|---|---|
| | | HPLC | | Sum of area % |
| | | Duplex | Sum of | for sense |
| | Assay, | purity, | impurities ≥ | and antisense |
| Description | mg/mL | Area % | 0.050 area % | strands |
| Control in vial | 99.3 | 98.7 | 1.3 | 89.0 |
| Luer-Lok ™ | 100.4 | 98.7 | 1.3 | 88.9 |
| syringe, slow | | | | |
| dispense | | | | |
| Luer-Lok ™ | 99.7 | 98.7 | 1.2 | 89.0 |
| syringe, fast | | | | |
| dispense | | | | |
| Insulin syringe, | 99.2 | 98.7 | 1.3 | 88.8 |
| slow dispense | | | | |
| Insulin syringe, | 99.8 | 98.7 | 1.3 | 89.0 |
| fast dispense | | | | |

Abbreviations:
AX-HPLC = anion exchange high performance liquid chromatography;
Control in vial = fitusiran drug product the vials intended for clinical study.

### Example 5: Storage Stability Analyses of Fitusiran Drug Product

Storage stability data for fitusiran drug product was collected at the recommended storage condition of 2-8° C., and at one or more accelerated conditions. The stability studies were designed in accordance with International Conference on Harmonisation of Technical Requirements for Registration of Pharmaceuticals for Human Use (ICH) Guideline QiA (R2) and the samples were stored in a container closure identical to the one used for storage of clinical material (i.e., 2 mL USP Type I glass vial with Teflon faced butyl rubber stopper). Stability data was collected as shown in Table 5.

TABLE 5
Stability Protocol for Fitusiran Drug Product

| | | Storage time (months) | | | | | | | | |
|---|---|---|---|---|---|---|---|---|---|---|
| Attribute | Acceptance Criteria | 0 | 1 | 3 | 6 | 9 | 12 | 18 | 24 | 36 |
| Appearance | Clear, colorless to pale yellow | S | SA | SA | SA | S | S | S | S | S |
| | solution essentially free of | | | | | | | | | |
| | particulates | | | | | | | | | |
| Purity non-denaturing IPRP-HPLC | | | | | | | | | | |
| Duplex | NLT 90.0 area % | S | SA | SA | SA | S | S | S | S | S |
| Single Strands | NMT 5.0 area % | | | | | | | | | |
| Impurities | Report sum of area % (XX) of | | | | | | | | | |
| | peaks of all peaks ≥ 0.050 area % | | | | | | | | | |
| Purity denaturing AX-HPLC | | | | | | | | | | |
| Total Single Strands | NLT 85.0 area % | S | SA | SA | SA | S | S | S | S | S |
| Impurities | Report sum of area % (XX) of | | | | | | | | | |
| | peaks of all peaks ≥ 0.050 area % | | | | | | | | | |
| Purity denaturing IPRP-HPLC | | | | | | | | | | |
| Total Single Strands | NLT 80.0 area % | S | SA | SA | SA | S | S | S | S | S |
| Impurities | Report sum of area % (0.0) of | | | | | | | | | |
| | peaks of all peaks ≥ 0.050 area % | | | | | | | | | |
| Assay | 90 to 110 mg/mL | S | SA | SA | SA | S | S | S | S | S |
| Osmolality | 240-390 mg/mL | S | SA | SA | SA | S | S | S | S | S |
| pH | 6.0-8.0 | S | SA | SA | SA | S | S | S | S | S |
| Particulate Matter | | | | | | | | | | |
| ≥10 μm | NMT 6,000 per container | S | | | A | | S | | S | S |
| ≥25 μm | NMT 600 per container | | | | | | | | | |
| Bacterial endotoxins | NMT 100 EU/mL | S | | | A | | S | | S | S |
| Sterility | Conforms to current | S | | | A | | | | | |
| | pharmacopeia | | | | | | | | | |
| Container closure integrity | No evidence of dye ingress | | | | | | S | | S | S |

S-Testing performed for samples stored at 2-8° C., and 25 ± 2° C./60 ± 5% RH
A-Testing performed for samples stored at 40 ± 2° C./75 ± 5% RH
Abbreviations: AX-HPLC = Anion exchange HPLC, IPRP-HPLC = Ion-pair reversed phase HPLC

The stability of fitusiran drug product was evaluated for trends using the following analytical procedures: visual appearance, assay by UV spectrophotometry, pH, osmolality, duplex purity by non-denaturing IPRP-HPLC, and single strand purity as measured by two orthogonal methods: purity by denaturing AX HPLC, and purity by denaturing IPRP-HPLC.

In order to further elucidate the thermal stability of the drug product, several stability studies included long term assessment of the drug product at 25° C./60% RH, as well as a 6-month accelerated aging study performed at 40° C./75% RH. The additional data (i.e., beyond 6 months at 25° C./60% RH, and all data collected at 40° C./75% RH) was collected in order to evaluate the suitability of the drug product for long term storage at the ICH Zone II climatic condition.

The storage stability data for three representative lots of Fitusiran drug product (P02314, P02715, and P07916) are provided in Tables 6-14, and summarized below.

At the recommended storage condition of 2-8° C., up to 36 months of stability data for fitusiran drug product have been collected. No significant changes have been identified for any parameter during storage at 2-8° C., indicating that this condition is suitable for long term storage of the drug product. Additionally, no significant changes have been observed during storage at 25° C./60% RH, or even during storage at 40° C./75% RH. Based upon this, and an evaluation of the suitability of the drug product for long-term storage at the ICH Zone II climatic condition (i.e., 25° C./60% RH), a 36-month shelf life has been assigned to fitusiran drug product stored at 2-8° C., and further confirmed by additional data collected since the time of the last update.

TABLE 6
Long-term stability 5° C. batch P02314
Drug Product: Fitusiran-solution for injection-100 mg/mL Batch no.: P02314
Batch size: 1.0 L
Containter/closure: Vial Manufacturier: AMRI
Storage condition: +5° C. ± 3° C.
Storage orientation: Upright

| | | Time | | | | | | | |
|---|---|---|---|---|---|---|---|---|---|
| | | Initial | 3 | 6 | 9 | 12 | 18 | 24 | 36 |
| Test item | Acceptance criteria | results | months | months | months | months | months | months | months |
| Appearance (visual inspection) | Clear, colorless to pale | Pass | Pass | Pass | Pass | Pass | Pass | Pass | Pass |
| | yellow solution essentially | | | | | | | | |
| | free of particulates | | | | | | | | |
| Duplex purity (IPRP HPLC UV | NLT 90.0 area % | 99.2 | 98.8 | 99.1 | 99.1 | 99.2 | 99.1 | 99.2 | 99.2 |
| (non-denaturing)) | | | | | | | | | |
| Single Strands | Report data (area %) | 0.4 | 0.4 | 0.3 | 0.4 | 0.4 | 0.6 | 0.8 | 0.5 |
| Impurities | Report sum of area % of | 0.4 | 0.6 | 0.4 | 0.4 | 0.3 | 0.4 | 0.0 | 0.3 |
| | all peaks ≥ 0.20 area % | | | | | | | | |
| Purity AX HPLC UV (denaturing) | | | | | | | | | |
| Sense Strand | Report data (area %) | 42.2 | 42.2 | 43.6 | 43.1 | 43.9 | 45.8 | 43.8 | 43.1 |
| Antisense Strance | | 48.7 | 48.5 | 50.1 | 50.2 | 50.9 | 51.9 | 50.9 | 50.5 |
| Total Single Strands | | 90.9 | 90.7 | 93.7 | 93.3 | 94.8 | 97.7 | 94.7 | 93.6 |
| Impurities | Report sum of area % of | 6.9 | 7.5 | 5.7 | 5.6 | 4.8 | 3.5 | 5.3 | 5.1 |
| | all peaks ≥ 0.20 area % | | | | | | | | |
| Purity (IPRP HPLC UV (denaturing)) | | | | | | | | | |
| Sense Strand | Report data (area %) | 40.3 | 39.1 | 40.7 | 41.1 | 42.3 | 39.7 | 41.2 | 40.9 |
| Antiense Strance | | 47.8 | 47.8 | 47.6 | 49.1 | 48.1 | 46.2 | 48.5 | 47.6 |
| Total Single Strands | | 88.1 | 86.9 | 88.3 | 90.2 | 90.4 | 85.9 | 89.7 | 88.5 |
| Impurities | Report sum of area % of | 10.1 | 11.8 | 10.9 | 8.4 | 8.9 | 13.1 | 9.5 | 10.5 |
| | all peaks ≥ 0.20 area % | | | | | | | | |
| Assay (UV absorption) | 90 to 110 mg/mL | 100 | 101 | 97 | 100 | 98 | 99 | 98 | 103 |
| pH (Ph. Eur. 2.2.3, USP <791>) | Report data | 6.9 | 7.0 | 6.9 | 6.9 | 6.8 | 6.9 | 6.9 | 7.1 |
| Osmolality | Report data (mOsm/kg) | 292 | 290 | 292 | 294 | 287 | 291 | 292 | 292 |
| (Ph. Eur. 2.2.35, USP <785>) | | | | | | | | | |
| Particulate matter (USP <788>) | | | | | | | | | |
| ≥10 μm | NMT 6,000 per container | 21 | NS | NS | NS | NS | NS | NS | 80 |
| ≥25 μm | NMT 600 per container | 0 | NS | NS | NS | NS | NS | NS | 2 |
| Bacterial endotoxins | NMT 100 EU/mL | 70 | NS | NS | NS | NS | NS | NS | 51 |
| Sterility | Conforms | Pass | NS | NS | NS | Pass | NS | Pass | Pass |

Abbreviations: NS = Not scheduled; ND = Not detected

TABLE 7
Long-term stability 25° C./60% RH batch P02314
Drug product: Fitusiran solution for injection-100 mg/mL Batch no.: P02314
Batch size: 1.0 L
Container/closure: Vial Manufacturier: AMRI
Storage condition: +25° C. ± 2° C./60% ± 5% RH
Storage orientation: Upright

| | | Time | | | | | | | |
|---|---|---|---|---|---|---|---|---|---|
| | | Initial | 3 | 6 | 9 | 12 | 18 | 24 | 36 |
| Test item | Acceptance criteria | results | months | months | months | months | months | months | months |
| Appearance (visual inspection) | Clear, colorless to pale | Pass | Pass | Pass | Pass | Pass | Pass | Pass | Pass |
| | yellow solution essentially | | | | | | | | |
| | free of particulates | | | | | | | | |
| Duplex purity (IPRP HPLC UV | NLT 90.0 area % | 99.2 | 98.8 | 99.0 | 99.1 | 99.3 | 99.2 | 99.2 | 99.2 |
| (non-denaturing)) | | | | | | | | | |
| Single Strands | Report data (area %) | 0.4 | 0.4 | 0.4 | 0.5 | 0.4 | 0.8 | 0.8 | 0.5 |
| Impurities | Report sum of area % of all | 0.4 | 0.6 | 0.5 | 0.4 | 0.3 | 0.0 | 0.0 | 0.3 |
| | peaks ≥ 0.20 area % | | | | | | | | |
| Purity AX HPLC UV (denaturing) | | | | | | | | | |
| Sense Strand | Report data (area %) | 42.2 | 42.2 | 42.9 | 43.0 | 43.8 | 46.2 | 44.3 | 43.1 |
| Antisense Strand | | 48.7 | 48.6 | 49.7 | 50.1 | 51.0 | 51.4 | 50.7 | 50.5 |
| Total Single Strands | | 90.9 | 90.8 | 92.6 | 93.1 | 94.8 | 97.6 | 95.0 | 93.6 |
| Impurities | Report sum of area % of all | 6.9 | 7.6 | 7.5 | 5.7 | 4.8 | 1.9 | 5.1 | 4.9 |
| | peaks ≥ 0.20 area % | | | | | | | | |
| Purity (IPRP HPLC UV (denaturing)) | | | | | | | | | |
| Sense Strand | Report data (area %) | 40.3 | 39.1 | 41.5 | 41.1 | 42.2 | 39.5 | 41.2 | 37.6 |
| Antisense Strand | | 47.8 | 47.9 | 48.7 | 49.1 | 48.0 | 46.1 | 48.6 | 47.8 |
| Total Single Strands | | 88.1 | 87.0 | 90.2 | 90.2 | 90.2 | 85.7 | 89.8 | 85.6 |
| Impurities | Report sum of are % of all | 10.1 | 11.7 | 8.9 | 8.6 | 9.1 | 13.4 | 9.5 | 13.0 |
| | peaks ≥ 0.20 area % | | | | | | | | |
| Assay (UV absorption) | 90 to 110 mg/mL | 100 | 101 | 97 | 100 | 99 | 99 | 100 | 107 |
| pH (Ph. Eur. 2.2.3, USP <791>) | Report data | 6.9 | 6.9 | 6.9 | 6.9 | 6.9 | 6.9 | 6.8 | 7.0 |
| Osmolality | Report data (mOsm/kg) | 292 | 291 | 293 | 296 | 292 | 294 | 294 | 294 |
| (Ph. Eur. 2.2.35, USP <785>) | | | | | | | | | |
| Particulate matter (USP <788>) | | | | | | | | | |
| ≥10 μm | NMT 6,000 per container | 21 | NS | NS | NS | NS | NS | NS | 90 |
| ≥25 μm | NMT 600 per container | 0 | NS | NS | NS | NS | NS | NS | 0 |
| Bacterial endotoxins | NMT 100 EU/mL | 70 | NS | NS | NS | NS | NS | NS | 26 |
| Sterility | Conforms | Pass | NS | NS | NS | Pass | NS | Pass | Pass |

Abbreviations: NS = Not scheduled; ND = Not detected

TABLE 8
Accelerated stability batch P02314

| Drug product: | Fitusiran-slution for injection-100 mg/mL | | Batch no.: | P02314 |
|---|---|---|---|---|
| Container | Vial | | Batch size: | 1.0 L |
| | | | Manufacturer: | AMRI |
| Storage condition: | +40° C. ± 2° C./75% ± 5% RH | | | |
| Storage orientation: | Upright | | | |
| | | Time | | |
| | Acceptance | Initial | 3 | 6 |
| Test item | criteria | results | months | months |
| Appearance (visual inspection) | Clear, colorless to pale yellow solution essentially | Pass | Pass | Pass |
| | free of particulates | | | |
| Duplex purity (IPRP HPLC UV | NLT 90.0 area % | 99.2 | 98.8 | 99.1 |
| (non-denaturing)) | | | | |
| Single Strands | Report data (area %) | 0.4 | 0.4 | 0.4 |
| Impurities | Report sum of area % of all peaks ≥ 0.20 area % | 0.4 | 0.7 | 0.4 |
| Purity AX HPLC UV (denaturing) | | | | |
| Sense Strand | Report data (area %) | 42.2 | 42.1 | 43.1 |
| Antisense Strand | | 48.7 | 48.7 | 50.3 |
| Total Single Strands | | 90.9 | 90.8 | 93.4 |
| Impurities | Report sum of area % of all peaks ≥ 0.20 area % | 6.9 | 7.2 | 6.8 |
| Purity (IPRP HPLC UV (denaturing)) | | | | |
| Sense Strand | Report data (area %) | 40.3 | 38.9 | 41.4 |
| Antisense Strand | | 47.8 | 47.9 | 49.6 |
| Total Single Strands | | 88.1 | 86.8 | 91.0 |
| Impurities | Report sum of area % of all peaks ≥ 0.20 area % | 10.1 | 12.1 | 8.0 |
| Assay (UV absorption) | 90 to 110 mg/mL | 100 | 100 | 97 |
| pH (Ph. Eur. 2.2.3, USP <791>) | Report data | 6.9 | 6.9 | 6.8 |
| Osmolality (Ph. Eur. 2.2.35, USP <785>) | Report data (mOsm/kg) | 292 | 292 | 295 |
| Particulate matter (USP <788>) | | | | |
| ≥10 μm | NMT 6.000 per container | 21 | NS | 20 |
| ≥25 μm | NMT 600 per container | 0 | NS | 0 |
| Bacterial endotoxins | NMT 100 EU/mL | 70 | NS | 33 |
| Sterility | Conforms | Pass | NS | Pass |

Abbreviations:
NS = Not scheduled

TABLE 9
Long-term stability 5° C. batch P02715
Drug product: Fitusiran solution for injection-100 mg/mL Batch no.: P02715
Batch size: 1.4 L
Container/closure: Vial Manufacturier: AMRI
Storage condition: +5° C. ± 3° C.
Storage orientation: Inverted

| | | Time | | | | | | | | |
|---|---|---|---|---|---|---|---|---|---|---|
| | | Initial | 1 | 3 | 6 | 9 | 12 | 18 | 24 | 36 |
| Test item | Acceptance criteria | results | month | months | months | months | months | months | months | months |
| Appearance (visual inspection) | Clear, colorless to pale | Pass | Pass | Pass | Pass | Pass | Pass | Pass | Pass | Pass |
| | yellow solution essentially | | | | | | | | | |
| | free of particulates | | | | | | | | | |
| Duplex purity (IPRP HPLC UV | NLT 90.0 area % | 99.2 | 99.1 | 99.2 | 99.3 | 99.2 | 99.1 | 98.5 | 99.1 | 99.1 |
| (non-denaturing)) | | | | | | | | | | |
| Single Strands | Report data (area %) | 0.5 | 0.9 | 0.8 | 0.7 | 0.8 | 0.9 | 0.6 | 0.5 | 0.5 |
| Impurities | Report some of area % of all | 0.3 | ND | ND | ND | ND | ND | 0.9 | 0.3 | 0.3 |
| | peaks ≥ 0.20 area % | | | | | | | | | |
| Purity AX HPLC UV (denaturing) | | | | | | | | | | |
| Sense Strand | Report data (area %) | 43.9 | 44.2 | 44.4 | 44.1 | 43.4 | 42.8 | 42.9 | 43.1 | 42.6 |
| Antisense Strand | | 50.0 | 50.8 | 50.6 | 50.8 | 49.9 | 49.3 | 50.0 | 50.5 | 49.8 |
| Total Single Strands | | 93.9 | 95.0 | 95.0 | 94.9 | 93.3 | 92.1 | 92.9 | 93.6 | 92.4 |
| Impurities | Report sum of area % of all | 5.6 | 4.9 | 4.3 | 4.9 | 5.7 | 6.5 | 6.0 | 5.2 | 6.6 |
| | peaks ≥ 0.20 area % | | | | | | | | | |
| Purity (IPRP HPLC UV (denaturing)) | | | | | | | | | | |
| Sense Strand | Report data (area %) | 41.0 | 4.5 | 40.6 | 41.8 | 41.1 | 41.2 | 40.3 | 42.5 | 38.9 |
| Antisense Strand | | 47.7 | 47.6 | 48.0 | 48.0 | 49.0 | 48.5 | 46.7 | 47.7 | 45.7 |
| Total Single Strands | | 88.7 | 88.1 | 88.6 | 89.8 | 90.1 | 69.7 | 87.0 | 90.2 | 84.6 |
| Impurities | Report sum of area % of all | 10.3 | 10.7 | 10.8 | 8.7 | 9.1 | 9.4 | 11.7 | 9.0 | 13.4 |
| | peaks ≥ 0.20 area % | | | | | | | | | |
| Assay (UV absorption) | 90 to 110 mg/mL | 99 | NT^{a} | 98 | 97 | 100 | 100 | 98 | 108 | 101 |
| pH (Ph. Eur. 2.2.3, USP <791>) | Report data | 6.9 | 7.0 | 6.9 | 6.9 | 6.9 | 7.0 | 7.0 | 6.9 | 6.9 |
| Osmolality | Report data (mOsm/kg) | 295 | 292 | 291 | 290 | 293 | 292 | 292 | 291 | 291 |
| (Ph. Eur. 2.2.35, USP <785>) | | | | | | | | | | |
| Particulate matter (USP <788>) | | | | | | | | | | |
| ≥10 μm | NMT 6,000 per container | 50 | NS | NS | NS | NS | 12 | NS | 827 | 10 |
| ≥25 μm | NMT 600 per container | 2 | NS | NS | NS | NS | 0 | NS | 37 | 0 |
| Bacterial endotoxins | NMT 100 EU/mL | 41 | NS | NS | NS | NS | 25 | NS | 37 | 37 |
| Sterility | Conforms | Pass | NS | NS | NS | NS | NS | NS | NS | Pass |
| Container Closure Integrity | No evidence of dye ingress | NS | NS | NS | NS | NS | Pass | NS | Pass | NS |

Abbreviations: NS = Not scheduled; ND = Not detected, NT = Not tested
^{a}see AMRI deviation D33015

TABLE 10
Long-term stability 25° C./60% RH batch P02715
Drug product: Fitusiran solution for injection-100 mg/mL Batch no.: P02715
Batch size: 1.4 L
Container/closure: Vial Manufacturier AMRI
Storage condition: +25° C. ± 2° C./60% ± 5% RH
Storage orientation: Inverted

| | | Time | | | | | | | | |
|---|---|---|---|---|---|---|---|---|---|---|
| | | Initial | 1 | 3 | 6 | 9 | 12 | 18 | 24 | 36 |
| Test item | Acceptance criteria | results | month | months | months | months | months | months | months | months |
| Appearance (visual inspection) | Clear, colorless to pale | Pass | Pass | Pass | Pass | Pass | Pass | Pass | Pass | Pass |
| | yellow solution essentially | | | | | | | | | |
| | free of particulates | | | | | | | | | |
| Duplex purity (IPRP HPLC UV | NLT 90.0 area % | 99.2 | 99.2 | 99.3 | 99.3 | 99.1 | 99.2 | 98.5 | 99.1 | 99.1 |
| (non-denaturing)) | | | | | | | | | | |
| Single Strands | Report data (area %) | 0.5 | 0.8 | 0.7 | 0.7 | 0.9 | 0.8 | 0.6 | 0.5 | 0.5 |
| Impurities | Report sum of area % of all | 0.3 | ND | <0.20 | <0.20 | <0.20 | ND | 0.9 | 0.3 | 0.3 |
| | peaks ≥ 0.20 area % | | | | | | | | | |
| Purity AX HPLC UV (denaturing) | | | | | | | | | | |
| Sense Strand | Report data (area %) | 43.9 | 44.0 | 44.2 | 43.7 | 43.5 | 42.9 | 42.9 | 43.1 | 42.7 |
| Antisense Strand | | 50.0 | 50.9 | 50.6 | 50.9 | 49.8 | 50.2 | 50.2 | 50.6 | 50.1 |
| Total Single Strands | | 93.9 | 94.9 | 94.8 | 94.6 | 93.3 | 93.1 | 93.1 | 93.7 | 92.8 |
| Impurities | Report sum of area % of all | 5.6 | 4.9 | 4.8 | 5.3 | 5.2 | 55.5 | 5.5 | 4.7 | 5.5 |
| | peaks ≥ 0.20 area % | | | | | | | | | |
| Purity (IPRP HPLC UV (denaturing)) | | | | | | | | | | |
| Sense Strand | Report data (area %) | 41.0 | 40.6 | 40.8 | 41.1 | 41.2 | 41.0 | 40.6 | 42.5 | 39.0 |
| Antisense Strand | | 47.7 | 47.7 | 48.2 | 48.4 | 49.1 | 48.3 | 47.0 | 47.9 | 46.0 |
| Total Single Strands | | 88.7 | 88.3 | 89.0 | 89.5 | 90.3 | 89.3 | 87.6 | 90.5 | 85.0 |
| Impurities | Report sum of area % of all | 10.3 | 10.5 | 10.2 | 9.2 | 9.0 | 9.5 | 11.5 | 9.0 | 12.9 |
| | peaks ≥ 0.20 area % | | | | | | | | | |
| Assay (UV absorption) | 90 to 110 mg/mL | 99 | NT | 98 | 97 | 100 | 101 | 98 | 107 | 102 |
| pH (Ph. Eur. 2.2.3, USP <791>) | Report data | 6.9 | 7.0 | 6.9 | 6.9 | 6.9 | 6.9 | 6.9 | 6.9 | 6.9 |
| Osmolality | Report data (mOsm/kg) | 295 | 293 | 291 | 293 | 294 | 292 | 292 | 295 | 294 |
| (Ph. Eur. 2.2.35, USP <785>) | | | | | | | | | | |
| Particulate matter (USP <788>) | | | | | | | | | | |
| ≥10 μm | NMT 6,000 per container | 50 | NS | NS | NS | NS | 28 | NS | 12 | 7 |
| ≥25 μm | NMT 600 per container | 2 | NS | NS | NS | NS | 0 | NS | 0 | 0 |
| Bacterial endotoxins | NMT 100 EU/mL | 41 | NS | NS | NS | NS | 15 | NS | 21 | 21 |
| Sterility | Conforms | Pass | NS | NS | NS | NS | NS | NS | NS | Pass |
| Container Closure Integrity | No evidence of dye ingress | NS | NS | NS | NS | NS | Pass | NS | Pass | NS |

Abbreviations: NS = Not scheduled; ND = Not detected, NT = Not tested
^{a}see AMRI deviation D33015

TABLE 11

| | Accelerated stability batch P02715 | | | | |
|---|---|---|---|---|---|
| Drug product: | Fitusiran-slution for injection-100 mg/mL | | | Batch no.: | P02715 |
| Container | Vial | | | Batch size: | 1.4 L |
| | | | | Manufacturer: | AMRI |
| Storage condition: | +40° C. ± 2° C./75% ± 5% RH | | | | |
| Storage orientation: | Upright | | | | |
| | | Time | | | |
| | Acceptance | Initial | 1 | 3 | 6 |
| Test item | criteria | results | month | months | months |
| Appearance (visual inspection) | Clear, colorless to pale yellow solution essentially | Pass | Pass | Pass | Pass |
| | free of particulates | | | | |
| Duplex purity (IPRP HPLC UV | NLT 90.0 area % | 99.2 | 99.1 | 99.3 | 99.4 |
| (non-denaturing)) | | | | | |
| Single Strands | Report data (area %) | 0.5 | 0.5 | 0.9 | 0.7 |
| Impurities | Report sum of area % of all peaks ≥ 0.20 area % | 0.3 | ND | ND | ND |
| Purity AX HPLC UV (denaturing) | | | | | |
| Sense Strand | Report data (area %) | 43.9 | 44.1 | 43.8 | 43.7 |
| Antisense Strand | | 50.0 | 50.9 | 50.8 | 51.3 |
| Total Single Strands | | 93.9 | 95.0 | 94.6 | 95.0 |
| Impurities | Report sum of area % of all peaks ≥ 0.20 area % | 5.6 | 4.9 | 5.0 | 4.8 |
| Purity (IPRP HPLC UV (denaturing)) | | | | | |
| Sense Strand | Report data (area %) | 41.0 | 40.7 | 41.0 | 41.0 |
| Antisense Strand | | 47.7 | 47.6 | 48.6 | 48.0 |
| Total Single Strands | | 88.7 | 88.5 | 89.6 | 89.0 |
| Impurities | Report sum of area % of all peaks ≥ 0.20 area % | 10.3 | 10.1 | 10.0 | 10.0 |
| Assay (UV absorption) | 90 to 110 mg/mL | 99 | NT | 98 | 97 |
| pH (Ph. Eur. 2.2.3, USP <791>) | Report data | 6.9 | 7.0 | 6.8 | 6.8 |
| Osmolality (Ph. Eur. 2.2.35, USP <785>) | Report data (mOsm/kg) | 295 | 292 | 293 | 296 |
| Particulate matter (USP <788>) | | | | | |
| ≥10 μm | NMT 6.000 per container | 50 | NS | NS | 22 |
| ≥25 μm | NMT 600 per container | 2 | NS | NS | 1 |
| Bacterial endotoxins | NMT 100 EU/mL | 41 | NS | NS | 7 |
| Sterility | Conforms | Pass | NS | NS | Pass |

Abbreviations:
NS = Not scheduled

TABLE 14

| | Accelerated stability batch P07916 | | | | |
|---|---|---|---|---|---|
| Drug product: | Fitusiran-slution for injection-100 mg/mL | | | Batch no.: | P07916 |
| Container | Vial | | | Batch size: | 2.0 L |
| | | | | Manufacturer: | AMRI |
| Storage condition: | +40° C. ± 2° C./75% ± 5% RH | | | | |
| Storage orientation: | Upright | | | | |
| | | Time | | | |
| | Acceptance | Initial | 1 | 3 | 6 |
| Test item | criteria | results | month | months | months |
| Appearance (visual inspection) | Clear, colorless to pale yellow solution essentially | Pass | | | |
| | free of particulates | | | | |
| Duplex purity (IPRP HPLC UV | NLT 90.0 area % | 98.5 | 98.6 | 98.6 | 98.5 |
| (non-denaturing)) | | | | | |
| Single Strands | Report data (area %) | 0.7 | 0.8 | 0.7 | 0.8 |
| Impurities | Report sum of area % of all peaks ≥ 0.20 area % | 0.9 | 0.7 | 0.7 | 0.7 |
| Purity AX HPLC UV (denaturing) | | | | | |
| Sense Strand | Report data (area %) | 44.5 | 44.4 | 44.8 | 44.5 |
| Antisense Strand | | 50.3 | 50.3 | 50.9 | 50.6 |
| Total Single Strands | | 94.7 | 94.7 | 95.7 | 95.1 |
| Impurities | Report sum of area % of all peaks ≥ 0.20 area % | 5.1 | 5.1 | 4.1 | 4.6 |
| Purity (IPRP HPLC UV (denaturing)) | | | | | |
| Sense Strand | Report data (area %) | 42.0 | 42.1 | 42.7 | 44.2 |
| Antisense Strand | | 46.9 | 48.7 | 48.9 | 48.5 |
| Total Single Strands | | 86.9 | 90.8 | 91.6 | 92.7 |
| Impurities | Report sum of area % of all peaks ≥ 0.20 area % | 10.9 | 8.9 | 8.1 | 7.2 |
| Assay (UV absorption) | 90 to 110 mg/mL | 100 | 100 | 102 | 98 |
| pH (Ph. Eur. 2.2.3, USP <791>) | Report data | 7.1 | 6.9 | 7.0 | 6.8 |
| Osmolality (Ph. Eur. 2.2.35, USP <785>) | Report data (mOsm/kg) | 307 | 308 | 308 | 310 |
| Particulate matter (USP <788>) | | | | | |
| ≥10 μm | NMT 6.000 per container | 22 | NS | NS | 22 |
| ≥25 μm | NMT 600 per container | 1 | NS | NS | 3 |
| Bacterial endotoxins | NMT 100 EU/mL | 0.6 | NS | NS | <0.1 |
| Sterility | Conforms | Pass | NS | NS | Pass |

Abbreviations:
NS = Not scheduled

We claim:

1. A pharmaceutical composition for inhibiting expression of a Serpinc1 gene, comprising a double-stranded ribonucleic acid (dsRNA) agent at a concentration of about 50 mg/mL to about 200 mg/mL and phosphate buffered saline (PBS) at a concentration of about 1 mM to about 10 mM,
wherein the pH and the osmolality of the pharmaceutical composition are suitable for subcutaneous administration to a subject,
wherein the dsRNA agent has a sense strand consisting of the nucleotide sequence of 5′-GfsgsUfuAfaCfaCfCfAfuUfuAfcUfuCfaAf-3′ (SEQ ID NO:941) and an antisense strand consisting of the nucleotide sequence of 5′-usUfsgAfaGfuAfaAfuggUfgUfuAfaCfcsasg-3′ (SEQ ID NO:960),
wherein a, g, c, and u are 2′-O-methyl (2′-OMe) A, G, C, and U; Af, Gf, Cf, and Uf are 2′-fluoro A, G, C, U; and s is a phosphorothioate linkage, and
wherein a ligand is conjugated to the 3′ end of the sense strand via a linker, and wherein the ligand and the linker have the following structure: 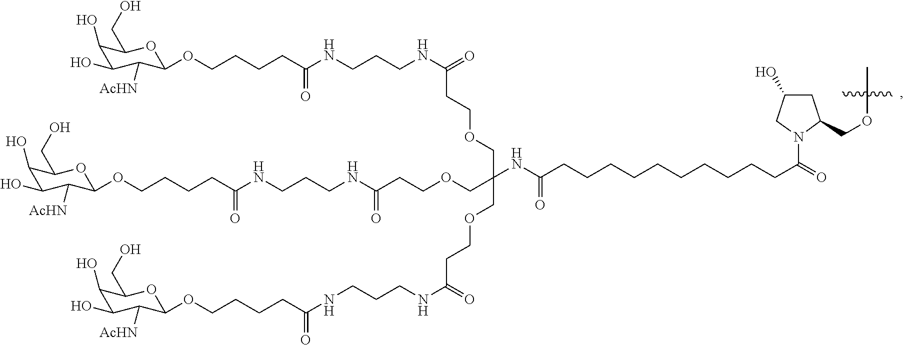 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]
wherein the dsRNA agent is in a free acid form.

2. A pharmaceutical composition for inhibiting expression of a Serpinc1 gene, comprising a double-stranded ribonucleic acid (dsRNA) agent at a concentration of about 50 mg/mL to about 200 mg/mL and phosphate buffered saline (PBS) at a concentration of about 1 mM to about 10 mM,
wherein the pH and the osmolality of the pharmaceutical composition are suitable for subcutaneous administration to a subject,
wherein the dsRNA agent has a sense strand consisting of the nucleotide sequence of 5′-GfsgsUfuAfaCfaCfCfAfuUfuAfcUfuCfaAf-3′ (SEQ ID NO:941) and an antisense strand consisting of the nucleotide sequence of 5′-usUfsgAfaGfuAfaAfuggUfgUfuAfaCfcsasg-3′ (SEQ ID NO:960),
wherein a, g, c, and u are 2′-O-methyl (2′-OMe) A, G, C, and U; Af, Gf, Cf, and Uf are 2′-fluoro A, G, C, U; and s is a phosphorothioate linkage, and
wherein a ligand is conjugated to the 3′ end of the sense strand via a linker, and wherein the ligand and the linker have the following structure: 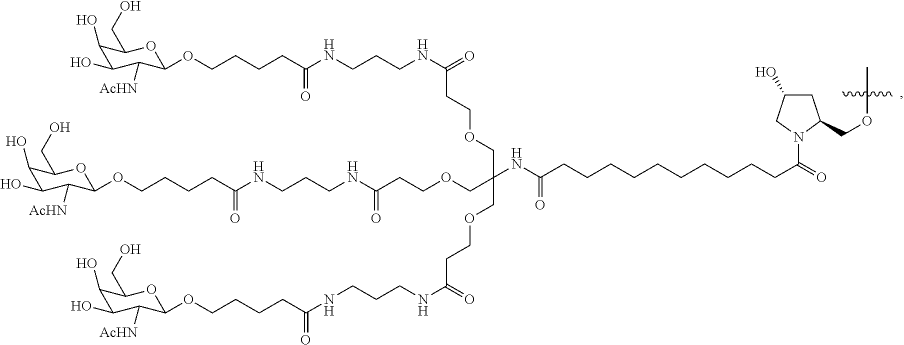 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]
wherein the dsRNA agent is in a salt form.

3. The pharmaceutical composition of claim 2, wherein the salt form is a sodium salt form.

4. The pharmaceutical composition of claim 3, wherein substantially all of the phosphodiester and/or phosphorothiotate groups in the agent comprise a sodium counterion.

5. The pharmaceutical composition of claim 3, wherein all of the phosphodiester and/or phosphorothiotate groups in the agent comprise a sodium counterion.

6. The pharmaceutical composition of any one of claims 1-4, wherein the concentration of PBS is between about 2 mM and about 7 mM.

7. The pharmaceutical composition of claim 6, wherein the concentration of PBS is about 3 to about 6 mM.

8. The pharmaceutical composition of claim 7, wherein the concentration of PBS is about 5 mM.

9. The pharmaceutical composition of any one of claims 1-8, wherein the pH of the composition is between about 5.0 to about 8.0.

10. The pharmaceutical composition of claim 9, wherein the pH of the composition is between about 6.0 to about 8.0.

11. The pharmaceutical composition of claim 10, wherein the pH of the composition is between about 6.5 to about 7.5.

12. The pharmaceutical composition of claim 11, wherein the pH of the composition is between about 6.8 to about 7.2.

13. The pharmaceutical composition of any one of claims 1-12, wherein the osmolality of the composition is between about 50 and about 400 mOsm/kg.

14. The pharmaceutical composition of claim 13, wherein the osmolality of the composition is between about 100 and about 400 mOsm/kg.

15. The pharmaceutical composition of claim 14, wherein the osmolality of the composition is between about 240 and about 390 mOsm/kg.

16. The pharmaceutical composition of claim 15, wherein the osmolality of the composition is between about 290 and about 320 mOsm/kg.

17. The pharmaceutical composition of any one of claims 1-16, wherein the concentration of the dsRNA agent in the pharmaceutical composition is between about 50 mg/mL and about 150 mg/mL.

18. The pharmaceutical composition of claim 17, wherein the concentration of the dsRNA agent in the pharmaceutical composition is between about 80 mg/mL and about 110 mg/mL.

19. The pharmaceutical composition of claim 18, wherein the concentration of the dsRNA agent in the pharmaceutical composition is about 100 mg/mL.

20. The pharmaceutical composition of any one of claims 1-19, wherein the composition is stable for up to about 36 months when stored at about 2° C. to about 8° C.

21. The pharmaceutical composition of any one of claims 1-19, wherein the composition is stable for up to about 36 months when stored at about 25° C. and 60% relative humidity (RH).

22. The pharmaceutical composition of any one of claims 1-19, wherein the composition is stable for up to about 6 months when stored at about 40° C. and 75% relative humidity (RH).

23. The pharmaceutical composition of any one of claims 1-22, wherein the composition comprises not less than (NLT) about 90.5 area % duplex and not more than (NMT) about 5 area % single strands as determined by purity non-denaturing IPRP-HPLC.

24. The pharmaceutical composition of any one of claims 1-22, wherein the composition comprises not less than (NLT) about 85.0 area % total single strands as determined by purity denaturing AX-HPLC.

25. The pharmaceutical composition of any one of claims 1-22, wherein the composition comprises not less than (NLT) about 80.0 area % total single strands as determined by purity denaturing IPRP-HPLC.

26. A vial comprising the pharmaceutical composition of any one of claims 1-25.

27. The vial of claim 26, wherein the vial comprises about 0.5 mL to about 2.0 ml of the pharmaceutical composition.

28. The vial of claim 27, wherein the vial comprises about 0.8 ml of the pharmaceutical composition.

29. A syringe comprising the pharmaceutical composition of any one of claims 1-25.

30. The syringe of claim 29, wherein the syringe is a 1 ml syringe.

31. The syringe of claim 29, wherein the syringe is a 3 ml syringe.

32. The syringe of any one of claims 29-31, wherein the syringe comprises a 29 G needle.

33. The syringe of any one of claims 29-31, wherein the syringe comprises a 30 G needle.

34. A pharmaceutical composition for inhibiting expression of a Serpinc1 gene, comprising a double-stranded ribonucleic acid (dsRNA) agent at a concentration of about 100 mg/mL and phosphate buffered saline (PBS) at a concentration of about 5 mM,
wherein the pH of the pharmaceutical composition is about 6.8 to about 7.2,
wherein the osmolality of the pharmaceutical composition is about 300 mOsm/kg,
wherein the dsRNA agent has a sense strand consisting of the nucleotide sequence of 5′-GfsgsUfuAfaCfaCfCfAfuUfuAfcUfuCfaAf-3′ (SEQ ID NO:941) and an antisense strand consisting of the nucleotide sequence of 5′-usUfsgAfaGfuAfaAfuggUfgUfuAfaCfcsasg-3′ (SEQ ID NO:960),
wherein a, g, c, and u are 2′-O-methyl (2′-OMe) A, G, C, and U; Af, Gf, Cf, and Uf are 2′-fluoro A, G, C, U; and s is a phosphorothioate linkage, and
wherein a ligand is conjugated to the 3′ end of the sense strand via a linker, and wherein the ligand and the linker have the following structure: 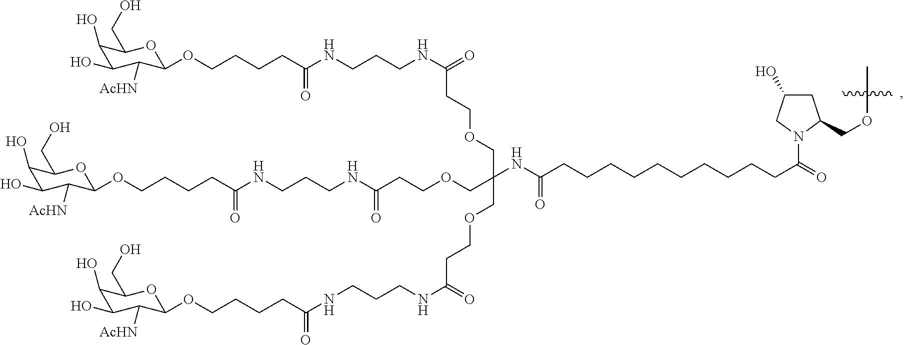 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]
wherein the dsRNA agent is in a free acid form.

35. A pharmaceutical composition for inhibiting expression of a Serpinc1 gene, comprising a double-stranded ribonucleic acid (dsRNA) agent at a concentration of about 106 mg/mL and phosphate buffered saline (PBS) at a concentration of about 5 mM,
wherein the pH of the pharmaceutical composition is about 6.8 to about 7.2,
wherein the osmolality of the pharmaceutical composition is about 300 mOsm/kg,
wherein the dsRNA agent has a sense strand consisting of the nucleotide sequence of 5′-GfsgsUfuAfaCfaCfCfAfuUfuAfcUfuCfaAf-3′ (SEQ ID NO:941) and an antisense strand consisting of the nucleotide sequence of 5′-usUfsgAfaGfuAfaAfuggUfgUfuAfaCfcsasg-3′ (SEQ ID NO:960),
wherein a, g, c, and u are 2′-O-methyl (2′-OMe) A, G, C, and U; Af, Gf, Cf, and Uf are 2′-fluoro A, G, C, U; and s is a phosphorothioate linkage, and
wherein a ligand is conjugated to the 3′ end of the sense strand via a linker, and wherein the ligand and the linker have the following structure: 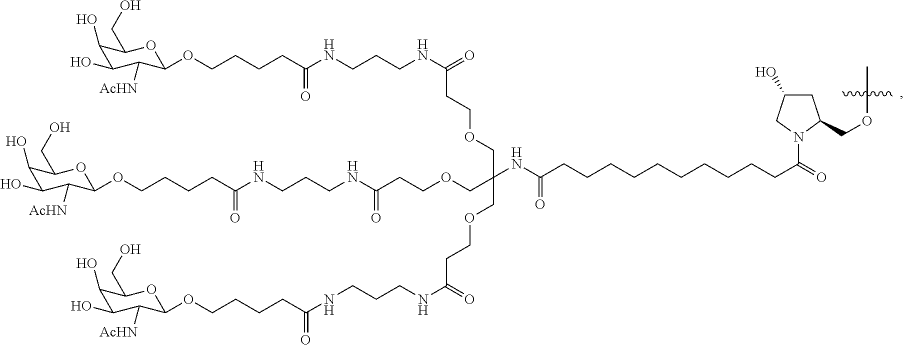 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]
wherein the dsRNA agent is in a salt form.

36. The pharmaceutical composition of claim 35, wherein the salt form is a sodium salt form.

37. The pharmaceutical composition of claim 36, wherein substantially all of the phosphodiester and/or phosphorothioate groups in the agent comprise a sodium counterion.

38. The pharmaceutical composition of claim 36, wherein all of the phosphodiester and/or phosphorothioate groups in the agent comprise a sodium counterion.

39. A pharmaceutical composition for inhibiting expression of a Serpinc1 gene, comprising a double-stranded ribonucleic acid (dsRNA) agent at a concentration of about 100 mg/mL and phosphate buffered saline (PBS) at a concentration of about 5 mM,
wherein the pH of the pharmaceutical composition is about 6.8 to about 7.2,
wherein the dsRNA agent has a sense strand consisting of the nucleotide sequence of 5′-GfsgsUfuAfaCfaCfCfAfuUfuAfcUfuCfaAf-3′ (SEQ ID NO:941) and an antisense strand consisting of the nucleotide sequence of 5′-usUfsgAfaGfuAfaAfuggUfgUfuAfaCfcsasg-3′ (SEQ ID NO:960),
wherein a, g, c, and u are 2′-O-methyl (2′-OMe) A, G, C, and U; Af, Gf, Cf, and Uf are 2′-fluoro A, G, C, U; and s is a phosphorothioate linkage, and
wherein a ligand is conjugated to the 3′ end of the sense strand via a linker, and wherein the ligand and the linker have the following structure: 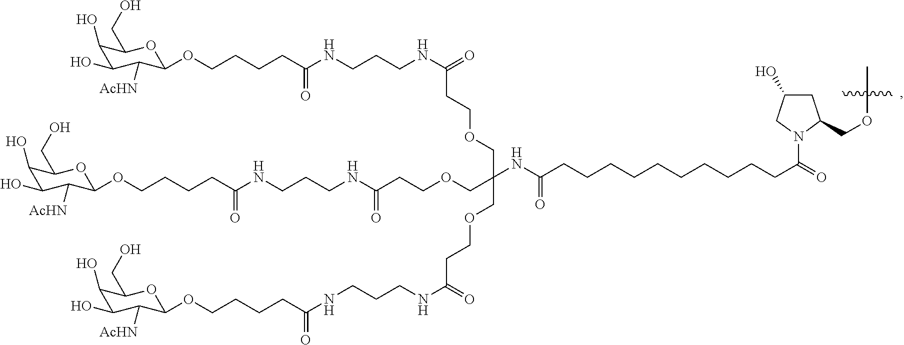 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]
wherein the dsRNA agent is in a free acid form.

40. A pharmaceutical composition for inhibiting expression of a Serpinc1 gene, comprising a double-stranded ribonucleic acid (dsRNA) agent at a concentration of about 106 mg/mL and phosphate buffered saline (PBS) at a concentration of about 5 mM,
wherein the pH of the pharmaceutical composition is about 6.8 to about 7.2,
wherein the dsRNA agent has a sense strand consisting of the nucleotide sequence of 5′-GfsgsUfuAfaCfaCfCfAfuUfuAfcUfuCfaAf-3′ (SEQ ID NO:941) and an antisense strand consisting of the nucleotide sequence of 5′-usUfsgAfaGfuAfaAfuggUfgUfuAfaCfcsasg-3′ (SEQ ID NO:960),
wherein a, g, c, and u are 2′-O-methyl (2′-OMe) A, G, C, and U; Af, Gf, Cf, and Uf are 2′-fluoro A, G, C, U; and s is a phosphorothioate linkage, and
wherein a ligand is conjugated to the 3′ end of the sense strand via a linker, and wherein the ligand and the linker have the following structure: 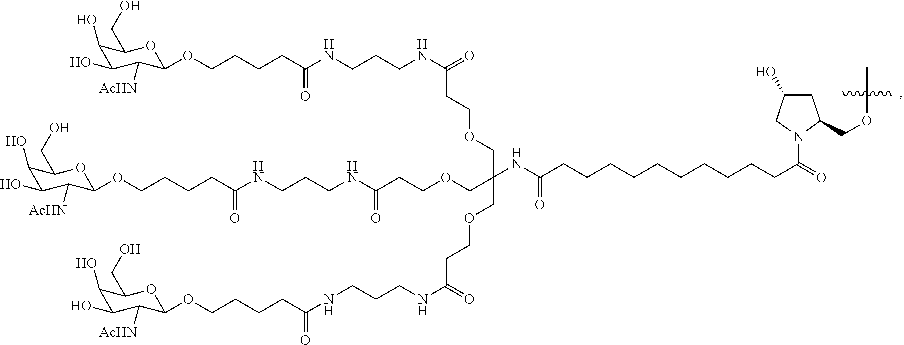 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]
wherein the dsRNA agent is in a salt form.

41. The pharmaceutical composition of any one of claims 34-40, wherein the composition is stable for up to about 36 months when stored at about 2° C. to about 8° C.

42. The pharmaceutical composition of any one of claims 34-40, wherein the composition is stable for up to about 36 months when stored at about 25° C. and 60% relative humidity (RH).

43. The pharmaceutical composition of any one of claims 34-40, wherein the composition is stable for up to about 6 months when stored at about 40° C. and 75% relative humidity (RH).

44. The pharmaceutical composition of any one of claims 34-40, wherein the composition comprises not less than (NLT) about 95.0 area % duplex and not more than (NMT) about 5 area % single strands as determined by purity non-denaturing IPRP-HPLC.

45. The pharmaceutical composition of any one of claims 34-44, wherein the composition comprises not less than (NLT) about 85.0 area % total single strands as determined by purity denaturing AX-HPLC.

46. The pharmaceutical composition of any one of claims 34-44, wherein the composition comprises not less than (NLT) about 80.0 area % total single strands as determined by purity denaturing IPRP-HPLC.

47. A vial comprising the pharmaceutical composition of any one of claims 34-46.

48. The vial of claim 47, wherein the vial comprises about 0.5 mL to about 2.0 ml of the pharmaceutical composition.

49. The vial of claim 48, wherein the vial comprises about 0.8 ml of the pharmaceutical composition.

50. A syringe comprising the pharmaceutical composition of any one of claims 34-46.

51. The syringe of claim 50, wherein the syringe is a 1 ml syringe.

52. The syringe of claim 50, wherein the syringe is a 3 ml syringe.

53. The syringe of any one of claims 50-52, wherein the syringe comprises a 29 G needle.

54. The syringe of any one of claims 50-52, wherein the syringe comprises a 30 G needle.

55. The syringe of any one of claims 50-52, wherein the syringe is a pre-filled syringe.
